(19) United States
(12) Patent Application Publication
(10) Pub. No.: US 2005/0009053 A1
(43) Pub. Date: Jan. 13, 2005

(54) Fragmentation-based methods and systems for de novo sequencing

(76) Inventors: Sebastian Boecker, Bielefeld (DE); Dirk van den Boom, La Jolla, CA (US)

Correspondence Address: Stephanie L. Seidman;FISH & RICHARDSON P.C., 12390 El Camino Real, San Diego, CA, 92130-2081, US

(21) Appl. No.: 10/830,943

(22) Filed: Apr. 22, 2004

Related U.S. Application Data
(60) Provisional application No. 60/466,006, filed on Apr. 25, 2003.

(51) Int. Cl. C12Q 1/68; G01N 33/53; G06F 19/00; G01N 33/48; G01N 33/50

(52) U.S. Cl. 435/6; 435/7.1; 702/20

(57) ABSTRACT

Methods and systems, particularly mass spectrometric methods and systems, for the analysis and sequencing of biomolecules, particularly nucleic acids, by fragmentation are provided.

This application claims the benefit of priority under 35 U.S.C. § 119(e) to U.S. Provisional Application Ser. No. 60/446,006, filed Apr. 25, 2003, entitled “Fragmentation-Based Methods and Systems for de novo Sequencing”, the subject matter and disclosure of which is incorporated herein in its entirety.

The subject matter and disclosure of each of the following applications also is incorporated herein by reference in its entirety: International PCT Application entitled “Fragmentation-Based Methods and Systems for de novo Sequencing”, filed Apr. 22, 2004, Attorney Docket number 17082-079WO1 (24736-2070PC), U.S. application Ser. No. 10/723,365, filed Nov. 26, 2003, entitled “Fragmentation-based Methods and Systems for Sequence Variation Detection and Discovery”, and International PCT Application Serial No. PCT/US03/37931, filed Nov. 26, 2003, entitled “Fragmentation-based Methods and Systems for Sequence Variation Detection and Discovery”.

### BACKGROUND

The genetic information of all living organisms (e.g., animals, plants and microorganisms) is encoded in deoxyribonucleic acid (DNA). In humans, the complete genome contains about 100,000 genes located on 24 chromosomes (The Human Genome, T. Strachan, BIOS Scientific Publishers, 1992). Each gene codes for a specific protein, which after its expression via transcription and translation, fulfils a specific biochemical function within a living cell.

A change or variation in the genetic code can result in a change in the sequence or level of expression of mRNA and potentially in the protein encoded by the mRNA. These changes, known as polymorphisms or mutations, can have significant adverse effects on the biological activity of the mRNA or protein resulting in disease. Mutations include nucleotide deletions, insertions, substitutions or other alterations (i.e., point mutations).

Many diseases caused by genetic polymorphisms are known and include hemophilias, thalassemias, Duchenne Muscular Dystrophy (DMD), Huntington's Disease (HD), Alzheimer's Disease and Cystic Fibrosis (CF) (Human Genome Mutations, D. N. Cooper and M. Krawczak, BIOS Publishers, 1993). Genetic diseases such as these can result from a single addition, substitution, or deletion of a single nucleotide in the deoxynucleic acid (DNA) forming the particular gene. In addition to mutated genes, which result in genetic disease, certain birth defects are the result of chromosomal abnormalities such as Trisomy 21 (Down's Syndrome), Trisomy 13 (Patau Syndrome), Trisomy 18 (Edward's Syndrome), Monosomy X (Turner's Syndrome) and other sex chromosome aneuploidies such as Klienfelter's Syndrome (XXY). Further, there is growing evidence that certain nucleic acid sequences can predispose an individual to any of a number of diseases such as diabetes, arteriosclerosis, obesity, various autoimmune diseases and cancer (e.g., colorectal, breast, ovarian, lung).

A change in a single nucleotide between genomes of more than one individual of the same species (e.g., human beings), that accounts for heritable variation among the individuals, is referred to as a single nucleotide polymorphism or “SNP.” Not all SNPs result in disease. The effect of an SNP, dependent on its position and frequency of occurrence, can range from harmless to fatal. Certain polymorphisms are thought to predispose some individuals to disease or are related to morbidity levels of certain diseases. Atherosclerosis, obesity, diabetes, autoimmune disorders, and cancer are a few of such diseases thought to have a correlation with polymorphisms. In addition to a correlation with disease, polymorphisms are also thought to play a role in a patient's response to therapeutic agents given to treat disease. For example, polymorphisms are believed to play a role in a patient's ability to respond to drugs, radiation therapy, and other forms of treatment.

Identifying polymorphisms can lead to better understanding of particular diseases and potentially more effective therapies for such diseases. Indeed, personalized therapy regimens based on a patient's identified polymorphisms can result in life saving medical interventions. Novel drugs or compounds can be discovered that interact with products of specific polymorphisms, once the polymorphism is identified and isolated. The identification of infectious organisms including viruses, bacteria, prions, and fingi, can also be achieved based on polymorphisms, and an appropriate therapeutic response can be administered to an infected host.

Complete genome sequences for a number of organisms, including humans, are currently available or are expected to become available in the near future. A parallel challenge is to characterize the types and extents of variation in the sequences, which in turn can be correlated to gene function, phenotype or identity (J. M. Blackwell, Trends Mol. Med. 7:521-526, 2001). As described above, the analysis of SNPs in particular will have an increasing impact on identification of human disease susceptibility genes and facilitate development of new drugs and patient care strategies. In addition, within the realm of (i) disease management; (ii) organism identification for, e.g., industrial, agricultural and forensic applications; and (iii) studying the regulation of gene expression, sequence information is necessary for the identification and typing of pathogens (e.g., bacteria, viruses and fungi), antibiotic or other drug-resistance profiling, determination of haplotypes, analysis of microsatellite sequences, STR (short tandem repeat) loci, allelic variation and/or frequency and the analysis of cellular methylation patterns.

Although a number of methods to monitor known sequence variations are known (see, e.g., for SNPs, U. Landegren et al., Genome Res., 8:769-776, 1998), these methods prove cumbersome and are subject to a high level of inaccuracy where the analysis of thousands of sequence variations is concerned. De novo sequence determination (i.e., determining the sequence without any a priori known sequence information) represents the ultimate level of resolution and sensitivity to identify which sequence variant or combination of sequence variants out of a large number of possible variants is present.

Two studies made the process of nucleic acid sequencing, at least with DNA, a common and relatively rapid procedure practiced in most laboratories. The first describes a process whereby terminally labeled DNA molecules are chemically cleaved in a base-specific manner (A. M. Maxam and W. Gilbert, Proc. Natl. Acad. Sci. USA 74:560-64, 1977). Each base position in the nucleic acid sequence is then determined from the molecular weights of fragments produced by base-specific cleavage. Individual reactions were devised to cleave preferentially at guanine, at adenine, at cytosine and thymine, and at cytosine alone. When the products of these four reactions are resolved by molecular weight, using, for example, polyacrylamide gel electrophoresis, DNA sequences can be read from the pattern of fragments on the resolved gel.

In another method, DNA is sequenced using a variation of the plus-minus method (Sanger et al. (1977) Proc. Natl. Acad. Sci. USA 74:5463-67, 1977). This procedure takes advantage of the chain terminating ability of dideoxynucleoside triphosphates (ddNTPs) and the ability of DNA polymerase to incorporate ddNTPs with nearly equal fidelity as the natural substrate of DNA polymerase, deoxynucleoside triphosphates (dNTPs). Briefly, a primer, usually an oligonucleotide, and a template DNA are incubated in the presence of a useful concentration of all four dNTPs plus a limited amount of a single ddNTP. The DNA polymerase occasionally incorporates a dideoxynucleotide that terminates chain extension. Because the dideoxynucleotide has no 3′-hydroxyl, the initiation point for the polymerase enzyme is lost. Polymerization produces a mixture of fragments of varied sizes, all having identical 3′ termini. Fractionation of the mixture by, for example, polyacrylamide gel electrophoresis, produces a pattern that indicates the presence and position of each base in the nucleic acid. Reactions with each of the four ddNTPs permits the nucleic acid sequence to be read from a resolved gel.

Mass spectrometry has been adapted and used for sequencing and detection of nucleic acid molecules (see, e.g., U.S. Pat. Nos. (6,194,144; 6,225,450; 5,691,141; 5,547,835; 6,238,871; 5,605,798; 6,043,031; 6,197,498; 6,235,478; 6,221,601; 6,221,605; see also P. Limbach, Mass. Spectrom. Rev., 15:297-336, 1996; K. Murray, J. Mass Spectrom., 31:1203-1215, 1996). In particular, Matrix-Assisted Laser Desorption/Ionization (MALDI) and ElectroSpray Ionization (ESI), which allow intact ionization, detection and exact mass determination of large molecules, i.e. well exceeding 300 kDa in mass, have been used for sequencing of nucleic acid molecules.

Mass spectrometry has also been adapted for sequencing of peptides (see, e.g., Dancik et al., J. Comp. Biol., 6:327-342, 1999; S. D. Patterson and R. Aebersold, Electrophoresis, 16:1791-1814, 1995). MALDI-MS requires incorporation of the macromolecule to be analyzed in a matrix, and has been performed on polypeptides and on nucleic acids mixed in a solid (i.e., crystalline) matrix. In these methods, a laser is used to strike the biopolymer/matrix mixture, which is crystallized on a probe tip, thereby effecting desorption and ionization of the biopolymer. In addition, MALDI-MS has been performed on polypeptides using the water of hydration (i, ice) or glycerol as a matrix. When the water of hydration was used as a matrix, it was necessary to first lyophilize or air dry the protein prior to performing MALDI-MS (Berkenkamp et al. (1996) Proc. Natl. Acad. Sci. USA 93:7003-7007). The upper mass limit for this method was reported to be 30 kDa with limited sensitivity (i.e., at least 10 pmol of protein was required).

A further refinement in mass spectrometric analysis of high molecular weight molecules was the development of time of flight mass spectrometry (TOF-MS) with matrix-assisted laser desorption ionization (MALDI). This process involves placing the sample into a matrix that contains molecules that assist in the desorption process by absorbing energy at the frequency used to desorb the sample. Time of flight analysis uses the travel time or flight time of the various ionic species as an accurate indicator of molecular mass. Since each of the four naturally occurring nucleotide bases, dC, dT, dA and dG, also referred to herein as C, T, A and G, in DNA has a different molecular weight: MC=289.2; MT=304.2; MA=313.2; MG=329.2; where MC, MT, MA, MG are average molecular weights in daltons of the nucleotide bases deoxycytidine, thymidine, deoxyadenosine, and deoxyguanosine, respectively, it is possible to read an entire sequence in a single mass spectrum. If a single spectrum is used to analyze the products of a conventional Sanger sequencing reaction, where chain termination is achieved at every base position by the incorporation of dideoxynucleotides, a base sequence can be determined by calculation of the mass differences between adjacent peaks. In addition, the method can be used to determine the masses, lengths and base compositions of mixtures of oligonucleotides and to detect target oligonucleotides based upon molecular weight.

MALDI-TOF mass spectrometry for sequencing nucleic acid using mass modification to increase mass resolution is available (see, e.g., U.S. Pat. Nos. 5,547,835; 6,194,144; 6,225,450; 5,691,141 and 6,238,871). The methods employ conventional Sanger sequencing reactions with each of the four dideoxynucleotides. In addition, for example for multiplexing, two of the four natural bases are replaced; dG is substituted with 7-deaza-dG and dA with 7-deaza-dA.

U.S. Pat. No. 5,622,824, describes methods for nucleic acid sequencing based on mass spectrometric detection. To achieve this, the nucleic acid is by means of protection, specificity of enzymatic activity, or immobilization, unilaterally degraded in a stepwise manner via exonuclease digestion and the nucleotides or derivatives detected by mass spectrometry. Prior to the enzymatic degradation, sets of ordered deletions that span a cloned nucleic acid fragment can be created. In this manner, mass-modified nucleotides can be incorporated using a combination of exonuclease and DNA/RNA polymerase. This permits either multiplex mass spectrometric detection, or modulation of the activity of the exonuclease so as to synchronize the degradative process.

Technologies have been developed to apply MALDI-TOF mass spectrometry to obtain sequence information on an industrial scale. These technologies can be applied to large numbers of either individual samples, or pooled samples to study allelic frequencies or the frequency of SNPs in populations of individuals, or in heterogeneous tumor samples. The analyses can be performed on chip-based formats in which the target nucleic acids or primers are linked to a solid support, such as a silicon or silicon-coated substrate, preferably in the form of an array (see, e.g., K. Tang et al., Proc. Natl. Acad. Sci. USA, 96:10016, 1999). Generally, when analyses are performed using mass spectrometry, particularly MALDI, small nanoliter volumes of sample are loaded onto a substrate such that the resulting spot is about, or smaller than, the size of the laser spot. It has been found that when this is achieved, the results from the mass spectrometric analysis are quantitative. The area under the signals in the resulting mass spectra are proportional to concentration (when normalized and corrected for background). Methods for preparing and using such chips are described in U.S. Pat. No. 6,024,925, co-pending U.S. application Ser. Nos. 08/786,988, 09/364,774, 09/371,150 and 09/297,575; see, also, U.S. application Ser. No. PCT/US97/20195, which published as WO 98/20020. Chips and kits for performing these analyses are commercially available from SEQUENOM, INC. under the trademarked MassARRAY® system. The MassARRAY® system relies on mass spectral analysis combined with the miniaturized array and MALDI-TOF (Matrix-Assisted Laser Desorption Ionization-Time of Flight) mass spectrometry to deliver results rapidly. It accurately distinguishes single base changes in the size of nucleic acid fragments associated with genetic variants without tags.

Although the use of MALDI for sequencing biomolecules has the potential of high throughput due to high-speed signal acquisition and automated analysis off solid surfaces, there are limitations in its application for the sequencing of large biomolecules. For example, in mass spectrometric sequencing methods that are based on sequence-specific extension and termination (i.e., a Sanger sequencing type approach), one limitation is their poor applicability to large nucleic acid molecules, e.g., to nucleic acid fragments beyond about 30-50 nucleotides (see, e.g., H. Koster et al., Nature Biotechnol., 14:1123-1128, 1996; WO 96/29431; WO 98/20166; WO 98/12355; U.S. Pat. No. 5,869,242; WO 97/33000; WO 98/54571). Mass spectrometry-based sequencing approaches that rely on fragmentation of larger molecules, e.g., nucleic acids of 300-500 or, in certain cases, up to 1000 nucleotides, essentially detect sequence variations that may in some cases be assigned to a polymorphism or mutation. While the masses of the fragments may be determined with sufficient accuracy to reduce the number of possible base compositions of each fragment, this data is often insufficient to unambiguously assemble the sequence of the entire target nucleic acid molecule, be it relative to a known reference nucleic acid (re-sequencing), or sequencing without any a-priori known information (de novo sequencing). Other sequencing approaches such as pyrosequencing (see, e.g., M. Ronaghi et al., Science, 281:363-365, 1998) or sequencing by hybridization (SBH) (see, e.g., R. Drmanac et al., Genomics, 4:114-128, 1989; W. Bains and G. C. Smith, J. Theor. Biol., 135:303-307, 1988; Y. Lysov et al., Dokl. Acad. Sci. USSR, 303:1508-1511, 1988) are also limited by the short sequencing length or, in the case of SBH, by the large number of false reads and the high cost of SBH chips.

Accordingly, a need exists for sequencing methods that can be used to sequence large biomolecules, that are time and cost-competitive, and that are accurate (low level of ambiguity) and robust. Because re-sequencing, or, more desirably, de novo sequencing approaches are the most sensitive and least ambiguous ways to obtain information on sequence variations and organism identity, there is a need for accurate, sensitive, precise and reliable methods for re-sequencing or de novo sequencing of biological macromolecules, particularly in connection with the diagnosis of conditions, diseases and disorders. Therefore, it is an object herein to provide sequencing methods that satisfy these needs and provide additional advantages.

### SUMMARY

Provided herein are methods and systems for sequencing and detecting nucleic acids and proteins using techniques, such as mass spectrometry and gel electrophoresis, that are based upon molecular mass. The methods and systems can be used for de novo sequencing; to identify genetic disease or chromosome abnormality; identify a predisposition to a disease or condition including, but not limited to, obesity, atherosclerosis, or cancer; identify an infection by an infectious agent; provide information relating to identity, heredity, or histocompatibility; identify pathogens (e.g., bacteria, viruses and fungi); provide antibiotic or other drug-resistance profiling; determine haplotypes; analyze microsatellite sequences and STR (short tandem repeat) loci; determine allelic variation and/or frequency; and analyze cellular methylation patterns.

Methods for sequencing long fragments of nucleic acid and proteins by specific and/or predictable fragmentation, such as by enzymatic cleavage, are provided. To perform such sequencing, partial fragmentation is achieved at a specific and/or predictable position in the nucleic acid or protein sequence based on (i) the base or amino acid specificity of the cleaving reagent (such as an endonuclease); or (ii) the structure and/or the chemical bonds of the target nucleic acid or protein molecule; or (iii) a combination of these, are generated from the target biomolecule. The analysis of fragments rather than the full length biomolecule shifts the mass of the ions to be determined into a lower mass range, which is generally more amenable to mass spectometric detection. For example, the shift to smaller masses increases mass resolution, mass accuracy and, in particular, the sensitivity for detection. The actual molecular weights of the fragments as determined by mass spectrometry provide sequence composition information. In one embodiment, the fragments generated are ordered to provide the sequence of the larger nucleic acid. The fragments are generated by partial cleavage, using a single specific cleavage reaction or complementary specific cleavage reactions such that alternative fragments of the same target biomolecule (e.g., a nucleic acid or polypeptide) sequence are obtained. The cleavage means may be enzymatic, chemical, physical or a combination thereof, so long as the target biomolecule is fragmented at specific and/or predictable cleavage sites on the target biomolecule.

One method of generating base specifically cleaved fragments from a nucleic acid is effected by contacting an appropriate amount of a target nucleic acid with an appropriate amount of a specific endonuclease for a specific length of time, thereby resulting in partial digestion of the target nucleic acid. Endonucleases will typically degrade a sequence into pieces of no more than about 50-70 nucleotides, even if the reaction is run to completion. In yet another method of generating base specifically cleaved partial fragments is the use of a mixture of cleavable and non-cleavable nucleotides during chain elongation (e.g., trascription or amplification) of the target at selected ratios to achieve the desired partial cleavage of the elongated product. The cleavage reactions can be run to completion and the amount of partial cleavage can be controlled as described herein by the ratio of cleavable to non-cleavable nucleotides used. In one embodiment, the nucleic acid is a ribonucleic acid and the endonuclease is a ribonuclease (RNase) selected from among: the G-specific RNase T₁, the A-specific RNase U₂, the AIU specific RNase PhyM, U/C specific RNase A, C specific chicken liver RNase (RNase CL3) or crisavitin. In another embodiment, the endonuclease is a restriction enzyme that cleaves at least one site contained within the target nucleic acid.

This provides a means for accurate detection and/or sequencing of a an oligonucleotide and is particularly advantageous for detecting or sequencing a plurality of target nucleic acid molecules in a single reaction using any technique that distinguishes products based upon molecular weight. The methods herein are particularly adapted for mass spectrometric analyses.

For example, the methods provided herein can comprise one or more partial cleavage reactions specific for a nucleic acid. In one embodiment, the cleavage reactions are incomplete and result in a mixture of all possible combinations of partially cleaved products, in additon to uncleaved target. For example, if an uncleaved target nucleic acid has 4 potential cleavage sites (e.g., cut bases) therein, then the resulting mixture of cleavage products can have any combination of fragments of the target resulting from a single cleavage at one, two, three or all of the 4 cleavage sites; double cleavage at any combination of 2 cleavage sites; triple cleavage at any combination of 3 cleavage sites; or cleavage at all 4 cleavage sites. The mass of the cleaved and uncleaved target sequence fragments can be determined using methods known in the art including but not limited to mass spectroscopy and gel electrophoresis, such as MALDI/TOF or ESI-TOF. Once the mass of the fragments is determined, one or more nucleic acid base compositions are determined for each fragment that are near or equal to the measured mass of each fragment. Cleavage reactions specific for all four bases can be used to generate data sets comprising the possible base compositions for each specifically cleaved fragment that near or equal the measured mass of each fragment. The ratio of cleaved to uncleaved cleavage sites (e.g., bases) can be less than 1:1.

The possible compositions (referred to herein as compomers) for each fragment can then be used to determine the sequence of the target nucleic acid sequence. For example, software or mathematical algorithms can be used to reconstruct the target sequence data from possible base compositions. The methods herein permit sequencing of nucleic acid fragments of any size, particularly in the range of less than about 500 nt, more typically in the range of about 50 to about 250 nucleotides.

The methods provided herein are adaptable to any sequencing method or detection method that relies upon or includes fragmentation of nucleic acids. As discussed further below, fragmentation of polynucleotides is known in the art and can be achieved in many ways. For example, polynucleotides composed of DNA, RNA, analogs of DNA and RNA or combinations thereof, can be fragmented physically, chemically, or enzymatically. Fragments can vary in size, and suitable fragments are typically less that about 500 nucleic acids. In other embodiments, suitable fragments can fall within several ranges of sizes including but not limited to: less than about 200 bases, between about 50 to about 150 bases, betweein about 25 to about 75 bases; between about 3 to about 25 bases; between about 2 to about 15; or between about 1 to about 10; or any combination of these fragment sizes. In some aspects, fragments of about one or two nucleotides are utilized. Polynucleotides can be treated to form random fragments or specific fragments depending on the method of treatment used.

Fragmentation of nucleic acids can be used in combination with sequencing methods that rely on chain extension in the presence of chain-terminating nucleotides. These methods include, but are not limited to, sequencing methods based upon Sanger sequencing, and detection methods, such as primer oligo base extension (PROBE) (see, e.g., U.S. Pat. No. 6,043,031; allowed U.S. application Ser. No. 09/287,679; and U.S. Pat. No. 6,235,478), that rely on and include a step of chain extension.

In one embodiment, a single stranded DNA or RNA molecule is partially cleaved by a base specific (bio-)chemical reaction using, for example, RNAses or uracil-DNA-glycosylase (UDG). In partial cleavage, the cleavage reaction can be modified such that not all, but only a certain percentage of those bases are cleaved. In particular embodiments to achieve partial incomplete cleavage, the chemistry of the cleavage reaction can be modified such that not all of the ‘cut bases’ (like T for UDG) but only a certain percentage of the cut bases will be cleaved (see FIG. 12). For example, for UDG this can be achieved by employing a mixture of cleavable dTTP and non-cleavable dUTP during the PCR amplification of the target sequence under investigation. For RNAse T₁, this could be achieved by using a mixture of dGTP and rGTP in the transcription reaction (see FIG. 13). As a result, fragments containing zero, one, or more cut bases will appear with an intensity depending on the ratio of incorporated cleavable versus non-cleavable cut bases (for UDG, the ratio of dT versus dU offered in the PCR, corrected by some factor because of different incorporation rates for the “unnatural” nucleotide triphosphates used in either the PCR, primer extension or RNA transcription reaction).

Those skilled in the art will recognize that these methods are not limited to the use of only one cleavable nucleotide, and that further combinations are possible. Depending on the type of application, different biochemical or molecular biologic approaches may be chosen, either relying on enzymatic or chemical DNA or RNA based fragmentation.

There are several advantages provided herein for using partial, incomplete cleavage relative to the use of complete cleavage methods:

Focussing on partially cleaved fragments containing at most one cut base, the following numbers of fragments are obtained that can theoretically be discriminated by mass:

Fragment (F.) size in bases

| | 1 | 2 | 3 | 4 | 5 |
|---|---|---|---|---|---|
| F. containing no cut base | 3 | 6 | 10 | 15 | 21 |
| F. containing up to one cut base | 4 | 9 | 16 | 25 | 36 |

For example, using UDG the following six fragments of length two with no inner cut base: AA, AC, AG, CC, CG, GG can be distinguished. The numbers above provide upper bounds for those numbers encountered in practice. Under optimal circumstances, many more fragments can be distinguished with incomplete cleavage than with complete cleavage, lowering the risk that a fragment cannot be detected because another fragment with that mass already exists.

Another advantage stems from the supposition that a nucleotide fragment having length zero, one, or two bases would not give a peak detected by the mass spectrometer. Using incomplete cleavage, there is a high probability that one of the two fragments with one cut base ‘containing’ the original fragment will have length three or higher and, hence, its peak can be detected. For example, using the T-specific Uracil DNA Glycosylase (UDG) the oligo sequence ACATGTAGCTA (SEQ ID NO: 1) will create a fragment G when using complete cleavage that would not likely be detectable by mass spectrometry; but using the incomplete cleavage methods provided herein, the additional fragments ACATG and GTAGC would be obtained and detected.

Choosing an acceptable ratio between cleavable and non-cleavable cut bases is essential for obtaining a spectrum such that all ‘interesting’ peaks (most likely those from fragments containing none or one cut base) have high enough intensity, that is, signal-to-noise ratio. Simple theoretical calculations lead to a good estimate of a desired ratio: If the portion of cleaved cut bases is denoted x (so that the ratio of cleaved versus non-cleaved cut bases is x: (1−x)), we choose x=⅔ to maximize the predicted intensity of peaks corresponding to fragments containing exactly one non-cleaved cut base. Increasing x a little will increase the intensity of peaks corresponding to fragments containing no non-cleaved cut base, so x=0.7 is a good choice, leading to a ratio of 70% cleaved versus 30% non-cleaved cut bases.

In this case, peaks corresponding to fragments containing zero non-cleaved cut base will have approximately half the intensity of those of a spectrum from complete cleavage; peaks corresponding to fragments containing one non-cleaved cut base will have approximately 0.15 this intensity; while peaks corresponding to fragments containing two or more non-cleaved cut base will have less than 0.044 this intensity and will likely not be detected due to the noise of the spectrum. As a result, peaks corresponding to fragments containing none or one non-cleaved cut base will be detectable in the spectrum. In another embodiment, a ratio of 0.5 (i.e., 50% cleaved and 50% uncleaved) is desirbable because it maximizes peak intensities of fragments containing exactly one non-cleaved cut-base.

The resulting mixture of fragments is then analyzed using any method for mass detection (such as MALDI-TOF mass spectrometry), to acquire the molecular masses of the fragments. For every peak in the mass spectrum, the fragment base compositions (compomers) that will potentially create a peak of observed mass are determined. The partial cleavage reaction can be performed for all four bases to uniquely reconstruct the de novo underlying sequence from the molecular masses of the fragments. A single partial cleavage reaction can be performed, or complementary cleavage reactions can be performed. Complementary cleavage reactions refer to cleavage reactions that are carried out on the same target nucleic acid or protein using different cleavage reagents or by altering the cleavage specificity of the same cleavage reagent such that alternate cleavage patterns of the same target nucleic acid or protein are generated. In one embodiment, when the target is a nucleic acid, the complementary cleavage reactions are the four base-specific (A, G, C and T) cleavage reactions of the same target nucleic acid. The possible base compositions of the fragments are then ordered according to the number of specific cleavage sites that are not cleaved in each fragment due to the partial cleavage conditions. A sequencing graph corresponding to each cleavage reaction is constructed as a graph theoretical representation of the ordered compositions, and the sequencing graph(s) are traversed to reconstruct the underlying sequence information of the target biomolecule. Application of this method to simulated data indicates that it might be capable of sequencing nucleic acid molecules of greater than 200 bases.

An Exemplary Experimental Setup and Data Acquisition:

An exemplary experimental setup for the methods provided herein is as follows: A target molecule such as sample nucleic acid of an approximate length of 100-500 nucleotides is provided. Using polymerase chain reaction (PCR) or other amplification methods, the sample nucleic acid is multiplied. A single stranded target (either by transcription or other methods) is generated. Although the presented method can easily be extended to utilize double stranded data, single stranded data is utilized in the following.

In one embodiment, the target sample is DNA and in another the cleavage reaction might require transcription of the sample into RNA. The single stranded nucleic acid is cleaved with a base specific (bio-)chemical cleavage reaction: Such reactions cleave the amplicon sequence at exactly those positions where a specific base can be found. For example, amplification by PCR in the presence of dUTP, subsequent treatment with uracil-DNA-glycosylase (UDG) and fragmentation by alkaline treatment will cleave the sample DNA wherever dUTP was incorporated. (See e.g., Vaughan and McCarthy (1998), Nucleic Acids Research, 26(3):810-815; and McGrath et al., (1998), Anal. Biochem., 259(2):288-292). Such base specific cleavage can also be achieved by the use of RNAses, pn-bond cleavage, and other methods. The exact chemical results of these cleavage reactions are known in advance and can be simulated by an in silico experiment.

In one embodiment, the cleavage reaction is modified (by offering a mixture of cleavable versus non-cleavable “cut bases”) such that not all of these cut bases but only a certain percentage of them are cleaved. For example, offering a mixture of dUTP and dTTP during PCR with subsequent UDG cleavage will not cleave the sample nucleic acid whenever dTTP was incorporated. The resulting mixture contains all fragments that can be obtained from the sample nucleic acid by removing an arbitrary number of T's (see, e.g., FIG. 12). Such cleavage reactions are referred to herein as partial cleavage reactions.

Mass spectrometry, such as matrix assisted laser desorption ionization) TOF (time-of-flight) mass spectrometry (MS for short) is then applied to the products of the cleavage reaction, resulting in a sample spectrum that correlates mass and signal intensity of sample particles. The sample spectrum is analyzed to extract a list of signal peaks (with masses and intensities). For every such peak, one or more base compositions can be calculated (that is, nucleic acid molecules with unknown order but known multiplicity of bases) that could have created the detected peak, taking into account the inaccuracy of the mass spectrometry read. A list of base compositions (with intensities) is obtained depending on the sample nucleic acid and the incorporated cleavage method.

The above steps are repeated using cleavage reactions specific to all four bases. Alternatively, two suitably chosen cleavage reactions can be applied, once each to the forward and reverse strands. The result is four lists of base compositions, each one corresponding to a base specific cleavage reaction. The sample sequence can be uniquely reconstructed using the algorithms provided herein.

In another embodiment, the methods provided herein are used to analyze fragment data that comes from double stranded target nucleic acid. In this embodiment, two walks are simultaneously constructed in the respective sequencing graph, one (from first to last base) for the forward strand and another (from last to first base) for the reverse strand of the target DNA.

Other features and advantages will be apparent from the following detailed description and claims.

### BRIEF DESCRIPTION OF THE FIGURES

FIG. 1 is an exemplary undirected sequencing graph of order 1.

FIG. 2 is an exemplary directed sequencing graph of order 2.

FIG. 3 is an exemplary sequencing graph generated from compomers.

FIG. 4 is a flow diagram that illustrates an exemplary sequencing process according to an embodiment.

FIG. 5A and FIG. 5B form a flow diagram that illustrates an exemplary sequencing technique using sequencing graphs.

FIG. 6 illustrates an exemplary tabulated list of expected peaks (with at most one internal cut base) obtained from mass spectrometry, which is used to construct a sequencing graph.

FIG. 7 illustrates a distorted peak list and an interpretation of the list into compomers with no inner cut base and one inner cut base.

FIG. 8 is a sequencing graph reconstructed from the compomers (edges of the path corresponding to the sample sequence indicated by dashed lines) interpreted from the peak list shown in FIG. 7.

FIG. 9 is a block diagram of a system that performs sample processing and performs the operations illustrated in FIG. 4 and FIGS. 5A/5B.

FIG. 10 is a block diagram of a computer in the system of FIG. 9, illustrating the hardware components included in a computer that can provide the functionality of the stations and computers.

FIG. 11 is another exemplary directed sequencing graph of order 2.

FIG. 12 illustrates a exemplary resulting mixture containing all fragments that can be obtained from the sample DNA by removing an arbitrary number of T's by partial cleavage using UDG.

FIG. 13 illustrates a exemplary resulting mixture containing all fragments that can be obtained from sample DNA by partial cleavage using RNAse T1.

FIG. 14 illustrates the resulting mass spectrum of RNase A cleavage mediated fragmentation of RNA transcripts for partial incomplete cleavage at every T using a 80:20 mixture of dTTP:rUTP.

FIG. 15 illustrates the resulting mass spectrum of RNase A cleavage mediated fragmentation of RNA transcripts for complete cleavage using 100% dTTP.

FIG. 16 illustrates the resulting mass spectrum of UDG mediated fragmentation for incomplete cleavage using a 70:30 mixture of dUTP:dTTP.

FIG. 17 illustrates the resulting mass spectrum of UDG mediated fragmentation for complete cleavage using 100% dUTP.

FIG. 18 illustrates the resulting mass spectrum of UDG mediated fragmentation for the overlay of the incomplete cleavage spectrum (upper spectrum; FIG. 16) and the complete cleavage spectrum (lower spectrum; FIG. 17).

### DETAILED DESCRIPTION

A. Definitions
B. Methods of Generating Fragments
C. Sequencing Techniques by Construction of a Sequencing Graph
   1. Generation of Fragments by Partial Cleavage
   2. Construction of a Sequencing Graph
   3. Algorithm for Sequence Assembly from Fragments obtained by Partial Cleavage
D. Applications
E. System and Software Method
F. Examples
   A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, a molecule refers to any molecular entity and includes, but is not limited to, biopolymers, biomolecules, macromolecules or components or precursors thereof, such as peptides, proteins, organic compounds, oligonucleotides or monomeric units of the peptides, organics, nucleic acids and other macromolecules. A monomeric unit refers to one of the constituents from which the resulting compound is built. Thus, monomeric units include, nucleotides, amino acids, and pharmacophores from which small organic molecules are synthesized.

As used herein, a biomolecule is any molecule that occurs in nature, or derivatives thereof. Biomolecules include biopolymers and macromolecules and all molecules that can be isolated from living organisms and viruses, including, but are not limited to, cells, tissues, prions, animals, plants, viruses, bacteria, prions and other organsims. Biomolecules also include, but are not limited to oligonucleotides, oligonucleosides, proteins, peptides, amino acids, lipids, steroids, peptide nucleic acids (PNAs), oligosaccharides and monosaccharides, organic molecules, such as enzyme cofactors, metal complexes, such as heme, iron sulfur clusters, porphyrins and metal complexes thereof, metals, such as copper, molybedenum, zinc and others.

As used herein, macromolecule refers to any molecule having a molecular weight from the hundreds up to the millions. Macromolecules include, but are not limited to, peptides, proteins, nucleotides, nucleic acids, carbohydrates, and other such molecules that are generally synthesized by biological organisms, but can be prepared synthetically or using recombinant molecular biology methods.

As used herein, biopolymer refers to biomolecules, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein.

As used herein “nucleic acid” refers to polynucleotides such as deoxyribonucleic acid (DNA) and ribonucleic acid (RNA). The term should also be understood to include, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine. Reference to a nucleic acid as a “polynucleotide” is used in its broadest sense to mean two or more nucleotides or nucleotide analogs linked by a covalent bond, including single stranded or double stranded molecules. The term “oligonucleotide” also is used herein to mean two or more nucleotides or nucleotide analogs linked by a covalent bond, although those in the art will recognize that oligonucleotides such as PCR primers generally are less than about fifty to one hundred nucleotides in length. The term “amplifying,” when used in reference to a nucleic acid, means the repeated copying of a DNA sequence or an RNA sequence, through the use of specific or non-specific means, resulting in an increase in the amount of the specific DNA or RNA sequences intended to be copied.

As used herein, “nucleotides” include, but are not limited to, the naturally occurring DNA nucleoside mono-, di-, and triphosphates: deoxyadenosine mono-, di- and triphosphate; deoxyguanosine mono-, di- and triphosphate; deoxythymidine mono-, di- and triphosphate; and deoxycytidine mono-, di- and triphosphate (referred to herein as dA, dG, dT and dC or A, G, T and C, respectively). The term nucleotides also includes the naturally occurring RNA nucleoside mono-, di-, and triphosphates: adenosine mono-, di- and triphosphate; guanosine mono-, di- and triphosphate; uridine mono-, di- and triphosphate; and cytidine mono-, di- and triphosphate (referred to herein as rA, rG, rU and rC, respectively). Nucleotides also include, but are not limited to, modified nucleotides and nucleotide analogs such as deazapurine nucleotides, e.g., 7-deaza-deoxyguanosine (7-deaza-dG) and 7-deaza-deoxyadenosine (7-deaza-dA) mono-, di- and triphosphates, deutero-deoxythymidine (deutero-dT) mon-, di- and triphosphates, methylated nucleotides e.g., 5-methyldeoxycytidine triphosphate, ¹³C/¹⁵N labelled nucleotides and deoxyinosine mono-, di- and triphosphate. For those skilled in the art, it will be clear that modified nucleotides, isotopically enriched, depleted or tagged nucleotides and nucleotide analogs can be obtained using a variety of combinations of functionality and attachment positions.

As used herein, the phrase “chain-elongating nucleotides” is used in accordance with its art recognized meaning. For example, for DNA, chain-elongating nucleotides include 2′deoxyribonucleotides (e.g., dATP, dCTP, dGTP and dTTP) and chain-terminating nucleotides include 2′,3′-dideoxyribonucleotides (e.g., ddATP, ddCTP, ddGTP, ddTTP). For RNA, chain-elongating nucleotides include ribonucleotides (e.g., ATP, CTP, GTP and UTP) and chain-terminating nucleotides include 3′-deoxyribonucleotides (e.g., 3′dA, 3′dC, 3′dG and 3′dU) and 2′,3′-dideoxyribonucleotides (e.g., ddATP, ddCTP, ddGTP, ddTTP). A complete set of chain elongating nucleotides refers to dATP, dCTP, dGTP and dTTP for DNA, or ATP, CTP, GTP and UTP for RNA. The term “nucleotide” is also well known in the art.

As used herein, the term “nucleotide terminator” or “chain terminating nucleotide” refers to a nucleotide analog that terminates nucleic acid polymer (chain) extension during procedures wherein a DNA or RNA template is being sequenced or replicated. The standard chain terminating nucleotides, i.e., nucleotide terminators include 2′,3′-dideoxynucleotides (ddATP, ddGTP, ddCTP and ddTTP, also referred to herein as dideoxynucleotide terminators). As used herein, dideoxynucleotide terminators also include analogs of the standard dideoxynucleotide terminators, e.g., 5-bromo-dideoxyuridine, 5-methyl-dideoxycytidine and dideoxyinosine are analogs of ddTTP, ddCTP and ddGTP, respectively.

The term “polypeptide,” as used herein, means at least two amino acids, or amino acid derivatives, including mass modified amino acids, that are linked by a peptide bond, which can be a modified peptide bond. A polypeptide can be translated from a nucleotide sequence that is at least a portion of a coding sequence, or from a nucleotide sequence that is not naturally translated due, for example, to its being in a reading frame other than the coding frame or to its being an intron sequence, a 3′ or 5′ untranslated sequence, or a regulatory sequence such as a promoter. A polypeptide also can be chemically synthesized and can be modified by chemical or enzymatic methods following translation or chemical synthesis. The terms “protein,” “polypeptide” and “peptide” are used interchangeably herein when referring to a translated nucleic acid, for example, a gene product.

As used herein, a fragment of a biomolecule, such as biopolymer, refers to a smaller portion than the whole biomolecule. Fragments can contain from one constituent up to less than all. Typically when partially cleaving a target biomolecule, the resulting mixture of fragments will be of a plurality of different sizes such that most will contain more than two constituents (such as a constituent monomer); and the mixture of partially cleaved fragments can also include one or more copies of the full-length target biomolecule that has not undergone any cleavage.

As used herein, the term “fragments of a target nucleic acid” refers to cleavage fragments produced by specific and/or predictable physica cleavage, chemical cleavage or enzymatic cleavage of the target nucleic acid. As used herein, fragments obtained by specific and/or predictable cleavage refers to fragments that are cleaved at a specific and/or predictable position in a target nucleic acid sequence based on the base/sequence specificity of the cleaving reagent (e.g., A, G, C, T or U, or the recognition of modified bases or nucleotides); or the structure of the target nucleic acid; or physical processes, such as ionization of particular chemical bonds (covalent bonds) by collision-induced dissociation (e.g., either before or during mass spectrometry); or a combination thereof. Fragments can contain from one up to less than all of the constituent nucleotides of the traget nucleic acid molecule. The collection of fragments from such cleavage contains a variety of different size oligonucleotides and nucleotides, and the collection of fragments can include one or more copies of the full-length starting biomolecule that has not undergone any cleavage. Fragments can vary in size, and suitable nucleic acid fragments are typically less that about 2000 nucleotides. For example, suitable nucleic acid fragments can fall within several ranges of sizes including but not limited to: less than about 1000 bases; between about 100 to about 500 bases; from about 25 to about 200 bases; from about 3 to about 25 bases; or any combination of these fragment sizes. In some aspects, fragments of about one or two nucleotides may be present in the set of fragments obtained by specific cleavage.

As used herein, a target nucleic acid refers to any nucleic acid of interest in a sample. It can contain one or more nucleotides. A target nucleotide sequence refers to a particular sequence of nucleotides in a target nucleic acid molecule. Detection or identification of such sequence results in detection of the target and can indicate the presence or absence of a particular mutation, sequence variation, or polymorphism. Similarly, a target polypeptide as used herein refers to any polypeptide of interest whose mass is analyzed, for example, by using mass spectrometry to determine the amino acid sequence of at least a portion of the polypeptide, or to determine the pattern of peptide fragments of the target polypeptide produced, for example, by treatment of the polypeptide with one or more endopeptidases. The term “target polypeptide” refers to any polypeptide of interest that is subjected to mass spectrometry for the purposes disclosed herein, for example, for identifying the presence of a polymorphism or a mutation. A target polypeptide contains at least 2 amino acids, generally at least 3 or 4 amino acids, and particularly at least 5 amino acids. A target polypeptide can be encoded by a nucleotide sequence encoding a protein, which can be associated with a specific disease or condition, or a portion of a protein. A target polypeptide also can be encoded by a nucleotide sequence that normally does not encode a translated polypeptide. A target polypeptide can be encoded, for example, from a sequence of dinucleotide repeats or trinucleotide repeats or the like, which can be present in chromosomal nucleic acid, for example, a coding or a non-coding region of a gene, for example, in the telomeric region of a chromosome. The phrase “target sequence” as used herein refers to either a target nucleic acid sequence or a target polypeptide or protein sequence.

A process as disclosed herein also provides a means to identify a target polypeptide by mass spectrometric analysis of peptide fragments of the target polypeptide. As used herein, the term “peptide fragments of a target polypeptide” refers to cleavage fragments produced by specific chemical or enzymatic degradation of the polypeptide. The production of such peptide fragments of a target polypeptide is defined by the primary amino acid sequence of the polypeptide, since chemical and enzymatic cleavage occurs in a sequence specific manner. Peptide fragments of a target polypeptide can be produced, for example, by contacting the polypeptide, which can be immobilized to a solid support, with a chemical agent such as cyanogen bromide, which cleaves a polypeptide at methionine residues, or hydroxylamine at high pH, which can cleave an Asp-Gly peptide bond; or with an endopeptidase such as trypsin, which cleaves a polypeptide at Lys or Arg residues.

The identity of a target polypeptide can be determined by comparison of the molecular mass or sequence with that of a reference or known polypeptide. For example, the mass spectra of the target and known polypeptides can be compared.

As used herein, the term “corresponding or known polypeptide or nucleic acid” is a known polypeptide or nucleic acid generally used as a control to determine, for example, whether a target polypeptide or nucleic acid is an allelic variant of the corresponding known polypeptide or nucleic acid. It should be recognized that a corresponding known protein or nucleic acid can have substantially the same amino acid or base sequence as the target polypeptide, or can be substantially different. For example, where a target polypeptide is an allelic variant that differs from a corresponding known protein by a single amino acid difference, the amino acid sequences of the polypeptides will be the same except for the single amino acid difference. Where a mutation in a nucleic acid encoding the target polypeptide changes, for example, the reading frame of the encoding nucleic acid or introduces or deletes a STOP codon, the sequence of the target polypeptide can be substantially different from that of the corresponding known polypeptide.

As used herein, a reference biomolecule refers to a biomolecule, which is generally, although not necessarily, to which a target biomolecule is compared. Thus, for example, a reference nucleic acid is a nucleic acid to which the target nucleic acid is compared in order to identify potential or actual sequence variations in the target nucleic acid, or to type the target nucleic acid, relative to the reference nucleic acid. Reference nucleic acids typically are of known sequence or of a sequence that can be determined, such as by using the de novo sequencing methods provided herein.

As used herein, transcription-based processes include “in vitro transcription system”, which refers to a cell-free system containing an RNA polymerase and other factors and reagents necessary for transcription of a DNA molecule operably linked to a promoter that specifically binds an RNA polymerase. An in vitro transcription system can be a cell extract, for example, a eukaryotic cell extract. The term “transcription,” as used herein, generally means the process by which the production of RNA molecules is initiated, elongated and terminated based on a DNA template. In addition, the process of “reverse transcription,” which is well known in the art, is considered as encompassed within the meaning of the term “transcription” as used herein. Transcription is a polymerization reaction that is catalyzed by DNA-dependent or RNA-dependent RNA polymerases. Examples of RNA polymerases include the bacterial RNA polymerases, SP6 RNA polymerase, T3 RNA polymerase, T3 RNA polymerase, and T7 RNA polymerase.

As used herein, the term “translation” describes the process by which the production of a polypeptide is initiated, elongated and terminated based on an RNA template. For a polypeptide to be produced from DNA, the DNA must be transcribed into RNA, then the RNA is translated due to the interaction of various cellular components into the polypeptide. In prokaryotic cells, transcription and translation are “coupled”, meaning that RNA is translated into a polypeptide during the time that it is being transcribed from the DNA. In eukaryotic cells, including plant and animal cells, DNA is transcribed into RNA in the cell nucleus, then the RNA is processed into mRNA, which is transported to the cytoplasm, where it is translated into a polypeptide.

The term “isolated” as used herein with respect to a nucleic acid, including DNA and RNA, refers to nucleic acid molecules that are substantially separated from other macromolecules normally associated with the nucleic acid in its natural state. An isolated nucleic acid molecule is substantially separated from the cellular material normally associated with it in a cell or, as relevant, can be substantially separated from bacterial or viral material; or from culture medium when produced by recombinant DNA techniques; or from chemical precursors or other chemicals when the nucleic acid is chemically synthesized. In general, an isolated nucleic acid molecule is at least about 50% enriched with respect to its natural state, and generally is about 70% to about 80% enriched, particularly about 90% or 95% or more. Preferably, an isolated nucleic acid constitutes at least about 50% of a sample containing the nucleic acid, and can be at least about 70% or 80% of the material in a sample, particularly at least about 90% to 95% or greater of the sample. An isolated nucleic acid can be a nucleic acid molecule that does not occur in nature and, therefore, is not found in a natural state.

The term “isolated” also is used herein to refer to polypeptides that are substantially separated from other macromolecules normally associated with the polypeptide in its natural state. An isolated polypeptide can be identified based on its being enriched with respect to materials it naturally is associated with or its constituting a fraction of a sample containing the polypeptide to the same degree as defined above for an “isolated” nucleic acid, i.e., enriched at least about 50% with respect to its natural state or constituting at least about 50% of a sample containing the polypeptide. An isolated polypeptide, for example, can be purified from a cell that normally expresses the polypeptide or can produced using recombinant DNA methodology.

As used herein, “structure” of the nucleic acid includes but is not limited to secondary structures due to non-Watson-Crick base pairing (see, e.g., Seela, F. and A. Kehne (1987) Biochemistry, 26, 2232-2238.) and structures, such as hairpins, loops and bubbles, formed by a combination of base-paired and non base-paired or mis-matched bases in a nucleic acid.

As used herein, a “primer” refers to an oligonucleotide that is suitable for hybridizing, chain extension, amplification and sequencing. Similarly, a probe is a primer used for hybridization. The primer refers to a nucleic acid that is of low enough mass, typically about between about 5 and 200 nucleotides, generally about 70 nucleotides or less than 70, and of sufficient size to be conveniently used in the methods of amplification and methods of detection and sequencing provided herein. These primers include, but are not limited to, primers for detection and sequencing of nucleic acids, which require a sufficient number nucleotides to form a stable duplex, typically about 6-30 nucleotides, about 10-25 nucleotides and/or about 12-20 nucleotides. Thus, for purposes herein, a primer is a sequence of nucleotides contains of any suitable length, typically containing about 6-70 nucleotides, 12-70 nucleotides or greater than about 14 to an upper limit of about 70 nucleotides, depending upon sequence and application of the primer.

As used herein, reference to mass spectrometry encompasses any suitable mass spectrometric format known to those of skill in the art. Such formats include, but are not limited to, Matrix-Assisted Laser Desorption/Ionization, Time-of-Flight (MALDI-TOF), Electrospray ionization (ESi), IR-MALDI (see, e.g., published International PCT application No.99/57318 and U.S. Pat. No. 5,118,937), Orthogonal-TOF (O-TOF), Axial-TOF (A-TOF), Ion Cyclotron Resonance (ICR), Fourier Transform, Linear/Reflectron (RETOF), and combinations thereof. See also, Aebersold and Mann, Mar. 13, 2003, Nature, 422:198-207 (e.g., at FIG. 2) for a review of exemplary methods for mass spectrometry suitable for use in the methods provided herein, which is incorporated herein in its entirety by reference. MALDI, particular UV and IR, are among the preferred formats for mass spectrometry.

As used herein, mass spectrum refers to the presentation of data obtained from analyzing a biopolymer or fragment thereof by mass spectrometry either graphically or encoded numerically.

As used herein, pattern or fragmentation pattern or fragmentation spectrum with reference to a mass spectrum or mass spectrometric analyses, refers to a characteristic distribution and number of signals (such as peaks or digital representations thereof). In general, a fragmentation pattern as used herein refers to a set of fragments that are generated by specific cleavage of a biomolecule such as, but not limited to, nucleic acids and proteins. An unspecific reaction can be rendered specific by the use of modified building blocks. For example, an enzyme that specifically cleaves at both an A and C nucleotide can be rendered to specifically cleave at only the A nucleotide by using a modified uncleavable C nucleotide during amplification and/or transcription of the target sequence. Likewise, non-specific physical fragmentation can be rendered specific by the use of modified nucleic acids or amino acids, such that the the modified building blocks are less susceptible to fragmentation by the particular physical force being applied (e.g., an ionization force or a chemical reaction).

As used herein, signal, mass signal or output signal in the context of a mass spectrum or any other method that measures mass and analysis thereof refers to the output data, which is the number or relative number of molecules having a particular mass. Signals include “peaks” and digital representations thereof. It is well known that mass spectrometers measure “mass per charge” instead of the actual “mass” of the sample particles. However, because most particles that are detected via mass spectrometry are singly charged, those of skill in the art will recognize that the terms “mass” and “mass per charge” are used interchangeably. In addtion, because mass spectrometers (e.g., MALDI-TOF-MS) provide the “time-of flight” of the particles being analyzed, from which the mass is calculated (e.g., by a peak finding procedure), the calibration of the particular mass spectrometer used should be conducted before experimentation. Thus, for mass spectrometers that detect the time of fight for multiply charged particles (e.g., Electrospray Ionization), the mass is determined by dividing the mass obtained by the number of charges on the particle. Accordingly, each of the methods known in the art for detecting, determining, and/or calculating mass can be used for obtaining the mass encompassed by the methods provided herein.

As used herein, the term “peaks” refers to prominent upward projections from a baseline signal of a mass spectrometer spectrum (“mass spectrum”) which corresponds to the mass and intensity of a fragment. Peaks can be extracted from a mass spectrum by a manual or automated “peak finding” procedure.

As used herein, the mass of a peak in a mass spectrum refers to the mass computed by the “peak finding” procedure.

As used herein, the intensity of a peak in a mass spectrum refers to the intensity computed by the “peak finding” procedure that is dependent on parameters including, but not limited to, the height of the peak in the mass spectrum and its signal-to-noise ratio.

As used herein, “analysis” refers to the determination of certain properties of a single oligonucleotide or polypeptide, or of mixtures of oligonucleotides or polypeptides. These properties include, but are not limited to, the nucleotide or amino acid composition and complete sequence, the existence of single nucleotide polymorphisms and other mutations or sequence variations between more than one oligonucleotide or polypeptide, the masses and the lengths of oligonucleotides or polypeptides and the presence of a molecule or sequence within a molecule in a sample.

As used herein, “multiplexing” refers to the simultaneous determination of more than one oligonucleotide or polypeptide molecule, or the simultaneous analysis of more than one oligonucleotide or oligopeptide, in a single mass spectrometric or other mass measurement, i.e., a single mass spectrum or other method of reading sequence.

As used herein, the phrase, “a mixture of biological samples” refers to any two or more biomolecular sources that can be pooled into a single mixture for analysis herein. For example, the methods provided herein can be used for sequencing multiple copies of a target nucleic or amino acids from different sources, and therefore detect sequence variations in a target nucleic or amino acid in a mixture of nucleic acids in a biological sample. A mixture of biological samples can also include but is not limited to nucleic acid from a pool of individuals, or different regions of nucleic acid from one or more individuals, or a homogeneous tumor sample derived from a single tissue or cell type, or a heterogeneous tumor sample containing more than one tissue type or cell type, or a cell line derived from a primary tumor. Also contemplated are methods, such as haplotyping methods, in which two mutations in the same gene are detected.

As used herein, the term “amplifying” refers to means for increasing the amount of a biopolymer, especially nucleic acids. Based on the 5′ and 3′ primers that are chosen, amplification also serves to restrict and define the region of the genome which is subject to analysis. Amplification can be by any means known to those skilled in the art, including use of the polymerase chain reaction (PCR), etc. Amplification, e.g., PCR must be done quantitatively when the frequency of polymorphism is required to be determined.

As used herein, “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides in length. Thus, a polymorphism, e.g. genetic variation, refers to a variation in the sequence of a gene in the genome amongst a population, such as allelic variations and other variations that arise or are observed. Thus, a polymorphism refers to the occurrence of two or more genetically determined alternative sequences or alleles in a population. These differences can occur in coding and non-coding portions of the genome, and can be manifested or detected as differences in nucleic acid sequences, gene expression, including, for example transcription, processing, translation, transport, protein processing, trafficking, nucleic acid synthesis, expressed proteins, other gene products or products of biochemical pathways or in post-translational modifications and any other differences manifested amongst members of a population. A single nucleotide polymorphism (SNP) refers to a polymorphism that arises as the result of a single base change, such as an insertion, deletion or change (substitution) in a base.

A polymorphic marker or site is the locus at which divergence occurs. Such site can be as small as one base pair (an SNP). Polymorphic markers include, but are not limited to, restriction fragment length polymorphisms, variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different mendelian alleles for a gene. Polymorphisms can be observed by differences in proteins, protein modifications, RNA expression modification, DNA and RNA methylation, regulatory factors that alter gene expression and DNA replication, and any other manifestation of alterations in genomic nucleic acid or organelle nucleic acids.

As used herein, “polymorphic gene” refers to a gene having at least one polymorphic region.

As used herein, “allele”, which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has at least two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing amutation.

As used herein, “predominant allele” refers to an allele that is represented in the greatest frequency for a given population. The allele or alleles that are present in lesser frequency are referred to as allelic variants.

As used herein, changes in a nucleic acid sequence known as mutations can result in proteins with altered or in some cases even lost biochemical activities; this in turn can cause genetic disease. Mutations include nucleotide deletions, insertions or alterations/substitutions (i.e. point mutations). Point mutations can be either “missense”, resulting in a change in the amino acid sequence of a protein or “nonsense” coding for a stop codon and thereby leading to a truncated protein.

As used herein, the term “compomer” refers to the composition of a sequence fragment in terms of its monomeric component units. For nucleic acids, compomer refers to the base composition of the fragment with the monomeric units being bases; the number of each type of base can be denoted by Bₙ (ie: AₐC_{c}G_{g}Tₜ, with A₀C₀G₀T₀ representing an “empty” compomer or a compomer containing no bases). A natural compomer is a compomer for which all component monomeric units (e.g., bases for nucleic acids and amino acids for proteins) are greater than or equal to zero. For polypeptides, a compomer refers to the amino acid composition of a polypeptide fragment, with the number of each type of amino acid similarly denoted. A compomer corresponds to a sequence if the number and type of bases in the sequence can be added to obtain the composition of the compomer. For example, the compomer A₂G₃ corresponds to the sequence AGGAG. In general, there is a unique compomer corresponding to a sequence, but more than one sequence can correspond to the same compomer. For example, the sequences AGGAG, AAGGG, GGAGA, etc. all correspond to the same compomer A₂G₃, but for each of these sequences, the corresponding compomer is unique, i.e., A₂G₃.

As used herein, the “order k” of sequencing graphs (numerically denoted as 0, 1, 2, 3, 4, . . . ) refers to the maximum number of bases in the fragment that are not cleaved in a particular base-specific partial cleavage reaction. For example, for a sequence corresponding to AATGCACGTAGCCAGTCAAG (SEQ ID NO: 2), the order “0” for a T-specific cleavage reaction corresponds to cleavage at every single T in the sequence, the order “1” corresponds to fragments that have one uncleaved “T” (e.g., AATGCACG; GCACGTAGCCAG (SEQ ID NO: 3); etc.), the order “2” corresponds to fragments that have two uncleaved “T”s (e.g., AATGCACGTAGCCAG (SEQ ID NO: 4)).

As used herein, simulation (or simulating) refers to the calculation of a fragmentation pattern based on the sequence of a nucleic acid or protein and the predicted cleavage sites in the nucleic acid or protein sequence for a particular specific cleavage reagent. The fragmentation pattern can be simulated as a table of numbers (for example, as a list of peaks corresponding to the mass signals of fragments of a reference biomolecule), as a mass spectrum, as a pattern of bands on a gel, or as a representation of any technique that measures mass distribution. Simulations can be performed in most instances by a computer program.

As used herein, simulating cleavage refers to an in silico process in which a target molecule or a reference molecule is virtually cleaved.

As used herein, in silico refers to research and experminents performed using a computer. In silico methods include, but are not limited to, molecular modelling studies, biomolecular docking experiments, and virtual representions of molecular structures and/or processes, such as molecular interactions.

As used herein, a subject includes, but is not limited to, animals, plants, bacteria, viruses, parasites and any other organism or entity that has nucleic acid. Among subjects are mammals, preferably, although not necessarily, humans. A patient refers to a subject afflicted with a disease or disorder.

As used herein, a phenotype refers to a set of parameters that includes any distinguishable trait of an organism. A phenotype can be physical traits and can be, in instances in which the subject is an animal, a mental trait, such as emotional traits.

As used herein, “assignment” refers to a determination that the position of a nucleic acid or protein fragment indicates a particular molecular weight and a particular terminal nucleotide or amino acid.

As used herein, “plurality” refers to two or more polynucleotides or polypeptides, each of which has a different sequence. Such a difference can be due to a naturally occurring variation among the sequences, for example, to an allelic variation in a nucleotide or an encoded amino acid, or can be due to the introduction of particular modifications into various sequences, for example, the differential incorporation of mass modified nucleotides into each nucleic acid or protein in a plurality.

As used herein, an array refers to a pattern produced by three or more items, such as three or more loci on a solid support.

As used herein, a data processing routine refers to a process, that can be embodied in software, that determines the biological significance of acquired data (i.e., the ultimate results of the assay). For example, the data processing routine can make a genotype determination based upon the data collected. In the systems and methods herein, the data processing routine also controls the instrument and/or the data collection routine based upon the results determined. The data processing routine and the data collection routines are integrated and provide feedback to operate the data acquisition by the instrument, and hence provide the assay-based judging methods provided herein.

As used herein, “specifically hybridizes” refers to hybridization of a probe or primer only to a target sequence preferentially to a non-target sequence. Those of skill in the art are familiar with parameters that affect hybridization; such as temperature, probe or primer length and composition, buffer composition and salt concentration and can readily adjust these parameters to achieve specific hybridization of a nucleic acid to a target sequence.

As used herein, “sample” refers to a composition containing a material to be detected. In a preferred embodiment, the sample is a “biological sample.” The term “biological sample” refers to any material obtained from a living source, for example, an animal such as a human or other mammal, a plant, a bacterium, a fungus, a protist or a virus. The biological sample can be in any form, including a solid material such as a tissue, cells, a cell pellet, a cell extract, or a biopsy, or a biological fluid such as urine, blood, saliva, amniotic fluid, exudate from a region of infection or inflammation, or a mouth wash containing buccal cells, urine, cerebral spinal fluid and synovial fluid and organs. Preferably solid materials are mixed with a fluid. In particular, herein, the sample refers to a mixture of matrix used for mass spectrometric analyses and biological material such as nucleic acids. Derived from means that the sample can be processed, such as by purification or isolation and/or amplification of nucleic acid molecules.

As used herein, a composition refers to any mixture. It can be a solution, a suspension, liquid, powder, a paste, aqueous, non-aqueous or any combination thereof.

As used herein, a combination refers to any association between two or among more items.

As used herein, the term “1¼-cutter” refers to a restriction enzyme that recognizes and cleaves a 2 base stretch in the nucleic acid, in which the identity of one base position is fixed and the identity of the other base position is any three of the four naturally occurring bases.

As used herein, the term “1½-cutter” refers to a restriction enzyme that recognizes and cleaves a 2 base stretch in the nucleic acid, in which the identity of one base position is fixed and the identity of the other base position is any two out of the four naturally occurring bases.

As used herein, the term “2 cutter” refers to a restriction enzyme that recognizes and cleaves a specific nucleic acid site that is 2 bases long.

As used herein, the term “amplicon” refers to a region of nucleic acid that can be replicated.

As used herein, the term “partial cleavage”, “partial fragmentation” or “incomplete cleavage”, or grammatical variations thereof, refers to a reaction in which only a fraction of the respective cleavage sites for a particular cleavage reagent are actually cut by the cleavage reagent. The cleavage reagent can be, but is not limited to an enzyme; or a chemical or physical force. As set forth herein, one way of achieving partial cleavage is by using a mixture of cleavable or non-cleavable nucleotides or amino acids during target biomolecule production, such that the particular cleavage site contains uncleavable nucleotides or amino acids, which renders the target biomolecule partially cleaved, even when the cleavage reaction is run in an excess of time. For example, if an uncleaved target biomolecule has 4 potential cleavage sites (e.g., cut bases for a nucleic acid) therein, then the resulting mixture of cleavage products can have any combination of fragments of the target biomolecule resulting from: a single cleavage at one, two, three or all of the 4 cleavage sites; double cleavage at any one or more combinations of 2 cleavage sites; triple cleavage at any one or more combinations of 3 cleavage sites; or cleavage at all 4 cleavage sites.

As used herein, the term “complete cleavage” or “total cleavage” refers to a cleavage reaction in which all the cleavage sites recognized by a particular cleavage reagent are cut to completion, such that there are no internal “cut bases” within a cleaved fragment.

As used herein, the term “false positives” refers to additional mass signals within the mass spectra that are from background noise and not generated by specific actual or simulated cleavage of a nucleic acid or protein.

As used herein, the term “false negatives” refers to actual mass signals that are missing from an actual fragmentation spectrum but can be detected in the corresponding simulated spectrum.

As used herein, the term “cleave” or “cleavage” refers to any manner in which a nucleic acid or protein molecule is cut or fragmented into smaller pieces. The cleavage recognition sites can be one, two or more bases long; or can be particular bonds within a polynucleotide or polypeptide. The cleavage means include physical cleavage (such as shearing or collision induced fragmentation), enzymatic cleavage (such as with endonucleases), chemical cleavage (such as acid or base hydrolysis) and any other way smaller pieces of a nucleic acid are produced.

As used herein, cleavage conditions or cleavage reaction conditions refers to the set of one or more cleavage reagents or cleavage forces (such as chemical or physical forces described herein) that are used to perform actual or simulated cleavage reactions, and other parameters of the reactions including, but not limited to, time, temperature, pH, or choice of buffer.

As used herein, uncleaved cleavage sites refers to cleavage sites that are known recognition sites for a cleavage reagent but that are not cut by the cleavage reagent under the particular conditions of the reaction, e.g., modification of time, temperature, or the modification of the known bases at the cleavage recognition sites to prevent or reduce the likelihood of cleavage by the reagent.

As used herein, complementary cleavage reactions refers to cleavage reactions that are carried out or simulated on the same target or reference nucleic acid or protein using different cleavage reagents or by altering the cleavage specificity of the same cleavage reagent such that alternate cleavage patterns of the same target or reference nucleic acid or protein are generated.

As used herein, a combination refers to any association between two or more items or elements.

As used herein, fluid refers to any composition that can flow. Fluids thus encompass compositions that are in the form of semi-solids, pastes, solutions, aqueous mixtures, gels, lotions, creams and other such compositions.

As used herein, a cellular extract refers to a preparation or fraction which is made from a lysed or disrupted cell.

As used herein, a kit is a combination in which components are packaged optionally with instructions for use and/or reagents and apparatus for use with the combination.

As used herein, a system refers to the combination of elements with software and any other elements for controlling and directing methods provided herein.

As used herein, software refers to computer readable program instructions that, when executed by a computer, performs computer operations. Typically, software is provided on a program product containing program instructions recorded on a computer readable medium, such as but not limited to, magnetic media including floppy disks, hard disks, and magnetic tape; and optical media including CD-ROM discs, DVD discs, magneto-optical discs, and other such media on which the program instructions can be recorded.

As used herein, the term “backtracking” refers to a sequencing procedure in which potential components of the target sequence are linked according to some criteria until the requirements for completion are fulfilled or the process cannot continue along its current path, in which case a different path is tried, picking up from an earlier incomplete state of the current sequence or that of another sequence altogether.

As used herein, a deBruijn graph refers to a graph of vertices and edges in which each vertex represents a vector of elements and each edge represents a vector that is composed of those from the vertices it connects; you can model a sequence of elements, such as nucleotide bases, by tracing a path that uses each edge once (Eulerian), or visits each vertex once (Hamiltonian), or uses some other procedure, through the graph, if you set up the vertices and edges correctly.

As used herein, an Euler circuit for a given graph G is a circuit that contains every vertex and every edge of the graph. That is, an Euler circuit for a graph G is a sequence of adjacent vertices and edges in G that starts and ends at the same vertex, uses every vertex of G at least once, and uses every edge of G exactly once.

A Hamiltonian circuit for a given graph G is a simple circuit that includes every vertex of G. That is, a Hamiltonian circuit for G is a sequence of adjacent vertices and distinct edges in which every vertex of G appears exactly once.

As used herein, the term “sequencing graph” refers to a graph compriseing vertices and a set of edges where every edge connects exactly two vertices. In the methods provided herein, a list of peak masses and intensities is transformed into a proximity graph, also referred to herein as a “sequencing graph”. A graph is a mathematical construct composed of points called vertices and lines connecting the vertices called edges. Graphs can be used to model relationships, through the edges between vertices, and provide a convenient framework on which to structure efficient searching algorithms. In this case a ‘proximity’ graph can be built to represent cleaved sequence fragments as vertices and the adjacency of two such fragments in the full length target biomolecule (such as a nucleic acid) as edges between appropriate vertices.

As used herein, uncleaved “cut bases” means bases at which cleavage could have occurred under the reaction conditions but did not.

As used herein, a directed graph, such as a directed sequencing graph, is one in which travel along an edge proceeds from one vertex to another, but not vice-versa. This is represented by an edge drawn as an arrow.

As used herein, an undirected graph has edges drawn as lines with no arrowheads, since travel along an edge is not unidirectional, but can be in either direction between vertices. An undirected sequencing graph has the same properties as the directed sequencing graph, except that the edges are not directed (travel between two vertices is not restricted to one direction).

Definitions of the Algorithm Symbols

S an alphabet, or set of symbols which are used to compose strings
s=s₁ . . . sₙ a string of symbols, where each symbol is represented by sᵢ, i=1 . . . n
{<statement 1>: <statement 2>} a set of elements, a common property of which is described by statements 1 and 2, where statement 1 is qualified by statement 2; ‘:’ (or ‘|’) means ‘such that’ in this context
Sⁿ set of all strings formed from S of length n; {xy|x ε S, y ε S^{n-1}}
X ∪ Y ‘union’; a set that results from combining the elements of X and Y

$${S}^{+} {⋃}_{n = 1}^{∞} {S}^{n}$$

the set of all strings of any length greater than 0, formed from the alphabet S

$${S}^{*} {⋃}_{n = 0}^{∞} {S}^{n}$$

the set of all strings of any length, including 0, formed from the alphabet S
(a, b) ε (S^{*})² two elements a, b, each of which can be taken from the set S^{*} (they do not have to be the same) and used together
x ε S x is an element of S, which is a set of elements
S ⊂ S the set S is a subset of the set S
Gₖ(Cₓ, x) a subgraph of the de Bruijn graph of order k in which each vertex is a tuple of at most k number of elements; the tuple in this case is a set of compomers of sequentially contiguous DNA fragments separated from each other by the cut string x, which is not represented in the graph; vertices are connected by an edge only if the compomer represented by the edge can be shown likely to exist from the MS spectra
Gₖ(C_{ó}, ó) analogous to Gₖ(Cₓ, x) above, except that the cut string ó is a base-A,
C, G, or T
v^{start} a vertex that begins a walk in a graph
v^{end} a vertex that ends a walk in a graph
   |S|≦1ₘᵢₙ the length of the string s is greater than or equal to the minimum length measured for the sample sequence
   B. Methods of Generating Fragments
   Nucleic Acid Fragmentation

Fragmentation of nucleic acids is known in the art and can be achieved in many ways. For example, polynucleotides composed of DNA, RNA, analogs of DNA and RNA or combinations thereof, can be fragmented physically, chemically, or enzymatically, as long as the fragmentation is obtained by cleavage at a specific and predictable site in the target nucleic acid. Fragments can be cleaved at a specific position in a target nucleic acid sequence based on (i) the base specificity of the cleaving reagent (e.g., A, G, C, T or U, or the recognition of modified bases or nucleotides); or (ii) the structure of the target nucleic acid; or (iii) the physicochemical nature of a particular covalent bond between particular atoms of the nucleic acid; or a combination of any of these, are generated from the target nucleic acid. Fragments can vary in size, and suitable fragments are typically less that about 2000 nucleic acids. Suitable fragments can fall within several ranges of sizes including but not limited to: less than about 1000 bases, between about 100 to about 500 bases, from about 25 to about 200 bases, from about 3 to about 25 bases; or any combination of these sizes. In some aspects, fragments of about one or two nucleotides are desirable.

Accordingly, contemplated herein is specific and predictable physical fragmentation of nucleic acids or proteins using for example any physical force that can break one or more particular chemical bonds, such that a specific and predictable fragmentation pattern is produced. Such physical forces include but are not limited to Ionization radiation, such as X-rays, UV-rays, gamma-rays; dye-induced fragilization; chemical cleavage; or the like.

For example, in particular embodiments, polynucleotides can be fragmented by chemical reactions including for example, hydrolysis reactions including base and acid hydrolysis. Alkaline conditions can be used to fragment polyucleotides comprising RNA because RNA is unstable under alkaline conditions. See, e.g., Nordhoff et al. (1993) “Ion stability of nucleic acids in infrared matrix-assisted laser desorption/ionization mass spectrometry”, Nucl. Acids Res., 21(15):3347-57. DNA can be hydrolyzed in the presence of acids, typically strong acids such as 6M HCl. The temperature can be elevated above room temperature to facilitate the hydrolysis. Depending on the conditions and length of reaction time, the polynucleotides can be fragmented into various sizes including single base fragments. Hydrolysis can, under rigorous conditions, break both of the phosphate ester bonds and also the N-glycosidic bond between the deoxyribose and the purines and pyrimidine bases.

An exemplary acid/base hydrolysis protocol for producing polynucleotide fragments is described in Sargent et al. (1988) Methods Enzymol., 152:432. Briefly, 1 g of DNA is dissolved in 50 mL 0.1 N NaOH. 1.5 mL concentrated HCl is added, and the solution is mixed quickly. DNA will precipitate immediately, and should not be stirred for more than a few seconds to prevent formation of a large aggregate. The sample is incubated at room temperature for 20 minutes to partially depurinate the DNA. Subsequently, 2 mL 10 N NaOH(OH— concentration to 0.1 N) is added, and the sample is stirred until DNA redissolves completely. The sample is then incubated at 65° C. for 30 minutes to hydrolyze the DNA. Typical sizes range from about 250-1000 nucleotides but can vary lower or higher depending on the conditions of hydrolysis.

Another process whereby nucleic acid molecules are chemically cleaved in a base-specific manner is provided by A. M. Maxam and W. Gilbert, Proc. Natl. Acad. Sci. USA 74:560-64, 1977, and incorporated by reference herein. Individual reactions were devised to cleave preferentially at guanine, at adenine, at cytosine and thymine, and at cytosine alone.

Polynucleotides can also be cleaved via alkylation, particularly phosphorothioate-modified polynucleotides. K. A. Browne (2002) “Metal ion-catalyzed nucleic Acid alkylation and fragmentation”. J. Am. Chem. Soc. 124(27):7950-62. Alkylation at the phosphorothioate modification renders the polynucleotide susceptible to cleavage at the modification site. I. G. Gut and S. Beck describe methods of alkylating DNA for detection in mass spectrometry. I. G. Gut and S. Beck (1995) “A procedure for selective DNA alkylation and detection by mass spectrometry”. Nucleic Acids Res. 23(8): 1367-73. Another approach uses the acid lability of P3′-N5′-phosphoroamidate-containing DNA (Shchepinov et al., “Matrix-induced fragmentation of P3′-N5′-phosphoroamidate-containing DNA: high-throughput MALDI-TOF analysis of genomic sequence polymorphisms,” Nucleic Acids Res. 25: 3864-3872 (2001). Either dCTP or dTTP are replaced by their analog P-N modified nucleoside triphosphates and are introduced into the target sequence by primer extension reaction subsequent to PCR. Subsequent acidic reaction conditions produce base-specific cleavage fragments. In order to minimize depurination of adenine and guanine residues under the acidic cleavage conditions required, 7-deaza analogs of dA and dG can be used.

Single nucleotide mismatches in DNA heteroduplexes can be cleaved by the use of osmium tetroxide and piperidine, providing an alternative strategy to detect single base substitutions, generically named the “Mismatch Chemical Cleavage” (MCC) (Gogos et al., Nucl. Acids Res., 18: 6807-6817 [1990]).

Polynucleotide fragmentation can also be achieved by irradiating the polynucleotides. Typically, radiation such as gamma or x-ray radiation will be sufficient to fragment the polynucleotides. The size of the fragments can be adjusted by adjusting the intensity and duration of exposure to the radiation. Ultraviolet radiation can also be used. The intensity and duration of exposure can also be adjusted to minimize undesirable effects of radiation on the polynucleotides. Boiling polynucleotides can also produce fragments. Typically a solution of polynucleotides is boiled for a couple hours under constant agitation. Fragments of about 500 bp can be achieved. The size of the fragments can vary with the duration of boiling.

Polynucleotide fragments can result from enzymatic cleavage of single or multi-stranded polynucleotides. Multistranded polynucleotides include polynucleotide complexes comprising more than one strand of polynucleotides, including for example, double and triple stranded polynucleotides. Depending on the enzyme used, the polynucleotides are cut nonspecifically or at specific nucleotides sequences. Any enzyme capable of cleaving a polynucleotide can be used including but not limited to endonucleases, exonucleases, ribozymes, and DNAzymes. Enzymes useful for fragmenting polynucleotides are known in the art and are commercially available. See for example Sambrook, J., Russell, D. W., Molecular Cloning: A Laboratory Manual, the third edition, Cold Spring Harbor Laboratory Press, Cold Spring Harbor, N.Y., 2001, which is incorporated herein by reference. Enzymes can also be used to degrade large polynucleotides into smaller fragments.

Endonucleases are an exemplary class of enzymes useful for fragmenting polynucleotides. Endonucleases have the capability to cleave the bonds within a polynucleotide strand. Endonucleases can be specific for either double-stranded or single stranded polynucleotides. Cleavage can occur randomly within the polynucleotide or can cleave at specific sequences. Endonucleases which randomly cleave double strand polynucleotides often make interactions with the backbone of the polynucleotide. Specific fragmentation of polynucleotides can be accomplished using one or more enzymes is sequential reactions or contemporaneously. Homogenous or heterogenous polynucleotides can be cleaved. Cleavage can be achieved by treatment with nuclease enzymes provided from a variety of sources including the Cleavase® enzyme, Taq DNA polymerase, E. coli DNA polymerase I and eukaryotic structure-specific endonucleases, murine FEN-1 endonucleases [Harrington and Liener, (1994) Genes and Develop. 8:1344] and calf thymus 5′ to 3′ exonuclease [Murante, R. S., et al. (1994) J. Biol. Chem. 269:1191]). In addition, enzymes having 3′ nuclease activity such as members of the family of DNA repair endonucleases (e.g., the RrpI enzyme from Drosophila melanogaster, the yeast RAD1/RAD10 complex and E. coli Exo III), can also be used for enzymatic cleavage.

Restriction endonucleases are a subclass of endonucleases which recognize specific sequences within double-strand polynucleotides and typically cleave both strands either within or close to the recognition sequence. One commonly used enzyme in DNA analysis is HaeIII, which cuts DNA at the sequence 5′-GGCC-3′. Other exemplary restriction endonucleases include Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EciX I, EcOR I, EcOR I, EcOR II, EcOR V, Hae II, Hae III, Hind III, Hind III, Hpa I, Hpa II, Kpn I, Ksp I, Mlu I, MluN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I,,Sfi I, Sma I, Spe I, Sph I, Ssp I, Stu I, Sty I, Swa I, Taq I, Xba I, Xho I. The cleavage sites for these enzymes are known in the art.

Restriction enzymes are divided in types I, II, and III. Type I and type II enzymes carry modification and ATP-dependent cleavage in the same protein. Type III enzymes cut DNA at a recognition site and then dissociate from the DNA. Type I enzymes cleave a random sites within the DNA. Any class of restriction endonucleases can be used to fragment polynucleotides. Depending on the enzyme used, the cut in the polynucleotide can result in one strand overhanging the other also known as “sticky” ends. BamHI generates cohesive 5′ overhanging ends. KpnI generates cohesive 3′ overhanging ends. Alternatively, the cut can result in “blunt” ends that do not have an overhanging end. DraI cleavage generates blunt ends. Cleavage recognition sites can be masked, for example by methylation, if needed. Many of the known restriction endonucleases have 4 to 6 base-pair recognition sequences (Eckstein and Lilley (eds.), Nucleic Acids and Molecular Biology, vol. 2, Springer-Verlag, Heidelberg [1988]).

A small number of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167 [1987]). Recently, endonucleases encoded by group I introns have been discovered that might have greater than 12 base-pair specificity (Perlman and Butow, Science 246:1106 [1989]).

Restriction endonucleases can be used to generate a variety of polynucleotide fragment sizes. For example, CviJ1 is a restriction endonuclease that recognizes between a two and three base DNA sequence. Complete digestion with CviJ1 can result in DNA fragments averaging from 16 to 64 nucleotides in length. Partial digestion with CviJ1 can therefore fragment DNA in a “quasi” random fashion similar to shearing or sonication. CviJI normally cleaves RGCY sites between the G and C leaving readily cloneable blunt ends, wherein R is any purine and Y is any pyrimidine. However, in the presence of 1 mM ATP and 20% dimethyl sulfoxide the specificity of cleavage is relaxed and CviJI also cleaves RGCN and YGCY sites. Under these “star” conditions, CviJI cleavage generates quasi-random digests. Digested or sheared nucleic acid can be size selected at this point.

Methods for using restriction endonucleases to fragment polynucleotides are widely known in the art. In one exemplary protocol a reaction mixture of 20-50 μl is prepared containing: DNA 1-3 μg; restriction enzyme buffer 1X; and a restriction endonuclease 2 units for 1 μg of DNA. Suitable buffers are also known in the art and include suitable ionic strength, cofactors, and optionally, pH buffers to provide optimal conditions for enzymatic activity. Specific enzymes can require specific buffers which are generally available from commerical suppliers of the enzyme. An exemplary buffer is potassium glutamate buffer (KGB). Hannish, J. and M. McClelland. (1988). “Activity of DNA modification and restriction enzymes in KGB, a potassium glutamate buffer”, Gene Anal. Tech. 5:105; McClelland, M. et al. (1988) “A single buffer for all restriction endonucleases”, Nucleic Acid Res. 16:364. The reaction mixture is incubated at 37° C. for 1 hour or for any time period needed to produce fragments of a desired size or range of sizes. The reaction can be stopped by heating the mixture at 65° C. or 80° C. as needed. Alternatively, the reaction can be stopped by chelating divalent cations such as Mg²⁺ with for example, EDTA.

More than one enzyme can be used to fragment the polynucleotide. Multiple enzymes can be used in sequential reactions or in the same reation provided the enyzmes are active under similar conditions such as ionic strength, temperature, or pH. Typically, multiple enzymes are used with a standard buffer such as KGB. The polynucleotides can be partially or completely digested. Partially digested means only a subset of the restriction sites are cleaved. Complete digestion means all of the restriction sites are cleaved.

Endonucleases can be specific for certain types of polynucleotides. For example, endonuclease can be specific for DNA or RNA. Ribonuclease H is an endoribonuclease that specifically degrades the RNA strand in an RNA-DNA hybrid. Ribonuclease A is an endoribonuclease that specifically attacks single-stranded RNA at C and U residues. Ribonuclease A catalyzes cleavage of the phosphodiester bond between the 5′-ribose of a nucleotide and the phosphate group attached to the 3′-ribose of an adjacent pyrimidine nucleotide. The resulting 2′,3′-cyclic phosphate can be hydrolyzed to the corresponding 3′-nucleoside phosphate. RNase TI digests RNA at only G ribonucleotides and RNase U₂ digests RNA at only A ribonucleotides. The use of mono-specific RNases such as RNase T₁ (G specific) and RNase U₂ (A specific) has become routine (Donis-Keller et al., Nucleic Acids Res. 4: 2527-2537 (1977); Gupta and Randerath, Nucleic Acids Res. 4: 1957-1978 (1977); Kuchino and Nishimura, Methods Enzymol. 180: 154-163 (1989); and Hahner et al., Nuc. Acids Res. 25(10): 1957-1964 (1997)). Another enzyme, chicken liver ribonuclease (RNase CL3) has been reported to cleave preferentially at cytidine, but the enzyme's proclivity for this base has been reported to be affected by the reaction conditions (Boguski et al., J. Biol. Chem. 255: 2160-2163 (1980)). Recent reports also claim cytidine specificity for another ribonuclease, cusativin, isolated from dry seeds of Cucumis sativus L (Rojo et al., Planta 194: 328-338 (1994)). Alternatively, the identification of pyrimidine residues by use of RNase PhyM (A and U specific) (Donis-Keller, H. Nucleic Acids Res. 8: 3133-3142 (1980)) and RNase A (C and U specific) (Simoncsits et al., Nature 269: 833-836 (1977); Gupta and Randerath, Nucleic Acids Res. 4: 1957-1978 (1977)) has been demonstrated. In order to reduce ambiguities in sequence determination, additional limited alkaline hydrolysis can be performed. Since every phosphodiester bond is potentially cleaved under these conditions, information about omitted and/or unspecific cleavages can be obtained this way ((Donis-Keller et al., Nucleic Acids Res. 4: 2527-2537 (1977)). Benzonase® nuclease P1, and phosphodiesterase I are nonspecific endonucleases that are suitable for generating polynucleotide fragments ranging from 200 base pairs or less. Benzonase® is a genetically engineered endonuclease which degrades both DNA and RNA strands in many forms and is described in U.S. Pat. No. 5,173,418 which is incorporated by reference herein.

DNA glycosylases specifically remove a certain type of nucleobase from a given DNA fragment. These enzymes can thereby produce abasic sites, which can be recognized either by another cleavage enzyme, cleaving the exposed phosphate backbone specifically at the abasic site and producing a set of nucleobase specific fragments indicative of the sequence, or by chemical means, such as alkaline solutions and or heat. The use of one combination of a DNA glycosylase and its targeted nucleotide would be sufficient to generate a base specific signature pattern of any given target region.

Numerous DNA glcosylases are known. For example, a DNA glycosylase can be uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-ethenoadenine DNA glycosylase (see, e.g., U.S. Pat. Nos. 5,536,649; 5,888, 795; 5,952,176; 6,099,553; and 6,190,865 BI; International PCT application Nos. WO 97/03210, WO 99/54501; see, also, Eftedal et al. (1993) Nucleic Acids Res 21:2095-2101, Bjelland and Seeberg (1987) Nucleic Acids Res. 15:2787-2801, Saparbaev et al. (1995) Nucleic Acids Res. 23:3750-3755, Bessho (1999) Nucleic Acids Res. 27:979-983) corresponding to the enzyme's modified nucleotide or nucleotide analog target.

Uracil, for example, can be incorporated into an amplified DNA molecule by amplifying the DNA in the presence of normal DNA precursor nucleotides (e.g. dCTP, dATP, and dGTP) and dUTP. When the amplified product is treated with UDG, uracil residues are cleaved. Subsequent chemical treatment of the products from the UDG reaction results in the cleavage of the phosphate backbone and the generation of nucleobase specific fragments. Moreover, the separation of the complementary strands of the amplified product prior to glycosylase treatment allows complementary patterns of fragmentation to be generated. Thus, the use of dUTP and Uracil DNA glycosylase allows the generation of T specific fragments for the complementary strands, thus providing information on the T as well as the A positions within a given sequence. A C-specific reaction on both (complementary) strands (i.e., with a C-specific glycosylase) yields information on C as well as G positions within a given sequence if the fragmentation patterns of both amplification strands are analyzed separately. With the glycosylase method and mass spectrometry, a full series of A, C, G and T specific fragmentation patterns can be analyzed.

Several methods exist where treatment of DNA with specific chemicals modifies existing bases so that they are recognized by specific DNA glycosylases. For example, treatment of DNA with alkylating agents such as methylnitrosourea generates several alkylated bases including N3-methyladenine and N3-methylguanine which are recognized and cleaved by alkyl purine DNA-glycosylase. Treatment of DNA with sodium bisulfite causes deamination of cytosine residues in DNA to form uracil residues in the DNA which can be cleaved by uracil N-glycosylase (also known as uracil DNA-glycosylase). Chemical reagents can also convert guanine to its oxidized form, 8-hydroxyguanine, which can be cleaved by formamidopyrimidine DNA N-glycosylase (FPG protein) (Chung et al., “An endonuclease activity of Escherichia coli that specifically removes 8-hydroxyguanine residues from DNA,” Mutation Research 254: 1-12 (1991)). The use of mismatched nucleotide glycosylases have been reported for cleaving polynucleotides at mismatched nucleotide sites for the detection of point mutations (Lu, A-L and Hsu, I-C, Genomics (1992) 14, 249-255 and Hsu, I-C., et al, Carcinogenesis (1994)14, 1657-1662). The glycosylases used include the E. coli Mut Y gene product which releases the mispaired adenines of A/G mismatches efficiently, and releases A/C mismatches albeit less efficiently, and human thymidine DNA glycosylase which cleaves at Gfr mismatches. Fragments are produced by glycosylase treatment and subsequent cleavage of the abasic site.

Fragmentation of nucleic acids for the methods as provided herein can also be accomplished by dinucleotide (“2 cutter”) or relaxed dinucleotide (“1 and ½ cutter”, e.g.) cleavage specificity. Dinucleotide-specific cleavage reagents are known to those of skill in the art and are incorporated by reference herein (see, e.g., WO 94/21663; Cannistraro et al., Eur. J. Biochem., 181:363-370, 1989; Stevens et al., J. Bacteriol., 164:57-62, 1985; Marotta et al., Biochemistry, 12:2901-2904, 1973). Stringent or relaxed dinucleotide-specific cleavage can also be engineered through the enzymatic and chemical modification of the target nucleic acid. For example, transcripts of the target nucleic acid of interest can be synthesized with a mixture of regular and a-thio-substrates and the phosphorothioate internucleoside linkages can subsequently be modified by alkylation using reagents such as an alkyl halide (e.g., iodoacetamide, iodoethanol) or 2,3-epoxy-1-propanol. The phosphotriester bonds formed by such modification are not expected to be substrates for RNAses. Using this procedure, a mono-specific RNAse, such as RNAse-T1, can be made to cleave any three, two or one out of the four possible GpN bonds depending on which substrates are used in the a-thio form for target preparation. The repertoire of useful dinucleotide-specific cleavage reagents can be further expanded by using additional RNAses, such as RNAse-U2 and RNAse-A. In the case of RNAse A, for example, the cleavage specificity can be restricted to CpN or UpN dinucleotides through enzymatic incorporation of the 2′-modified form of appropriate nucleotides, depending on the desired cleavage specificity. Thus, to make RNAse A specific for CpG nucleotides, a transcript (target molecule) is prepared by incorporating aS-dUTP, aS-ATP, aS-CTP and GTP nucleotides. These selective modification strategies can also be used to prevent cleavage at every base of a homopolymer tract by selectively modifying some of the nucleotides within the homopolymer tract to render the modified nucleotides less resistant or more resistant to cleavage.

DNAses can also be used to generate polynucleotide fragments. Anderson, S. (1981) Shotgin DNA sequencing using cloned DNase I-generated fragments. Nucleic Acids Res. 9:3015-3027. DNase I (Deoxyribonuclease 1) is an endonuclease that digests double- and single-stranded DNA into poly- and mono-nucleotides. The enzyme is able to act upon single as well as double-stranded DNA and on chromatin.

Deoxyribonuclease type II is used for many applications in nucleic acid research including DNA sequencing and digestion at an acidic pH. Deoxyribonuclease II from porcine spleen has a molecular weight of 38,000 daltons. The enzyme is a glycoprotein endonuclease with dimeric structure. Optimum pH range is 4.5-5.0 at ionic strength 0.15 M. Deoxyribonuclease II hydrolyzes deoxyribonucleotide linkages in native and denatured DNA yielding products with 3′-phosphates. It also acts on p-nitrophenylphosphodiesters at pH 5.6-5.9. Ehrlich, S. D. et al. (1971) Studies on acid deoxyribonuclease IX 5′-Hydroxy-terminal and penultimate nucleotides of oligonucleotides obtained from calf thymus, deoxyribonucleic arid. Biochemistry. 10(11):2000-9.

Large single stranded polynucleotides can be fragmented into small polynucleotides using nuclease that remove various lengths of bases from the end of a polynuculeotide. Exemplary nucleases for removing the ends of single stranded polynucleotides include but are not limited to S1, Bal 31, and mung bean nucleases. For example, mung bean nuclease degrades single stranded DNA to mono or polynucleotides with phosphate groups at their 5′ termini. Double stranded nucleic acids can be digested completely if exposed to very large amounts of this enzyme.

Exonucleases are proteins that also cleave nucleotides from the ends of a polynucleotide, for example a DNA molecule. There are 5′ exonucleases (cleave the DNA from the 5′-end of the DNA chain) and 3′ exonucleases (cleave the DNA from the 3′-end of the chain). Different exonucleases can hydrolyse single-strand or double strand DNA. For example, Exonuclease III is a 3′ to 5′ exonuclease, releasing 5′-mononucleotides from the 3′-ends of DNA strands; it is a DNA 3′-phosphatase, hydrolyzing 3′-terminal phosphomonoesters; and it is an AP endonuclease, cleaving phosphodiester bonds at apurinic or apyrimidinic sites to produce 5′-termini that are base-free deoxyribose 5′-phosphate residues. In addition, the enzyme has an RNase H activity; it will preferentially degrade the RNA strand in a DNA-RNA hybrid duplex, presumably exonucleolytically. In mammalian cells, the major DNA 3′-exonuclease is DNase III (also called TREX-1). Thus, fragments can be formed by using exonucleases to degrade the ends of polynucleotides.

Catalytic DNA and RNA are known in the art and can be used to cleave polynucleotides to produce polynucleotide fragments. Santoro, S. W. and Joyce, G. F. (1997) A general purpose RNA-cleaving DNA enzyme. Proc. Natl. Acad. Sci. USA 94: 4262-4266. DNA as a single-stranded molecule can fold into three dimensional structures similar to RNA, and the 2′-hydroxy group is dispensable for catalytic action. As ribozymes, DNAzymes can also be made, by selection, to depend on a cofactor. This has been demonstrated for a histidine-dependent DNAzyme for RNA hydrolysis. U.S. Pat. Nos. 6,326,174 and 6,194,180 disclose deoxyribonucleic acid enzymes—catalytic or enzymatic DNA molecules—capable of cleaving nucleic acid sequences or molecules, particularly RNA. U.S. Pat. Nos. 6,265,167; 6,096,715; 5,646,020 disclose ribozyme compositions and methods and are incorporated herein by reference.

A DNA nickase, or DNase, can be used to recognize and cleave one strand of a DNA duplex. Numerous nickases are known. Among these, for example, are nickase NY2A nickase and NYSI nickase (Megabase) with the following cleavage sites:

| | |
|---|---|
| NY2A: | 5′...R AG...3′ |
| | 3′...Y TC...5′ |

where R=A or G and Y=C or T

| | |
|---|---|
| NYS1: | 5′... CC[A/G/T]...3′ |
| | 3′... GG[T/C/A]...5′. |

Subsequent chemical treatment of the products from the nickase reaction results in the cleavage of the phosphate backbone and the generation of fragments.

The Fen-1 fragmentation method involves the enzymes Fen-1 enzyme, which is a site-specific nuclease known as a “flap” endonuclease (U.S. Pat. Nos. 5,843,669, 5,874,283, and 6,090,606). This enzyme recognizes and cleaves DNA “flaps” created by the overlap of two oligonucleotides hybridized to a target DNA strand. This cleavage is highly specific and can recognize single base pair mutations, permitting detection of a single homologue from an individual heterozygous at one SNP of interest and then genotyping that homologue at other SNPs occurring within the fragment. Fen-1 enzymes can be Fen-1 like nucleases e.g. human, murine, and Xenopus XPG enzymes and yeast RAD2 nucleases or Fen-1 endonucleases from, for example, M jannaschii, P. furiosus, and P. woesei.

Another technique, which is under development as a diagnostic tool for detecting the presence of M. tuberculosis, can be used to cleave DNA chimeras. Tripartite DNA-RNA-DNA probes are hybridized to target nucleic acids, such as M. tuberculosis-specific sequences. Upon the addition of RNAse H, the RNA portion of the chimeric probe is degraded, releasing the DNA portions [Yule, Bio/Technology 12:1335 (1994)].

Fragments can also be formed using any combination of fragmentation methods as well as any combination of enzymes. Methods for producing specific fragments can be combined with methods for producing random fragments. Additionally, one or more enzymes that cleave a polynucleotide at a specific site can be used in combination with one or more enzymes that specifically cleave the polynucleotide at a different site. In another example, enzymes that cleave specific kinds of polynucleotides can be used in combination, for example, an RNase in combination with a DNase. In still another example, an enzyme that cleaves polynucleotides randomly can be used in combination with an enzymer that cleaves polynucleotides specifically. Used in combination means performing one or more methods after another or contemporaneously on a polynucleotide.

Peptide Fragmentation

As interest in proteomics has increased as a field of study, a number of techniques have been developed for protein fragmentation for use in protein sequencing. Among these are chemical and enzymatic hydrolysis, and fragmentation by ionization energy.

Sequential cleavage of the N-terminus of proteins is well known in the art, and can be accomplished using Edman degradation. In this process, the N-terminal amino acid is reacted with phenylisothiocyanate to for a PTC-protein with an intermediate anilinothiazolinone forming when contacted with trifluoroacetic acid. The intermediate is cleaved and converted to the phenylthiohydantoin form and subsequently separated, and identified by comparison to a standard. To facilitate protein cleavage, proteins can be reduced and alkylated with vinylpyridine or iodoacetamide.

Chemical cleavage of proteins using cyanogen bromide is well known in the art (Nikodem and Fresco, Anal. Biochem. 97: 382-386 (1979); Jahnen et al., Biochem. Biophys. Res. Commun. 166: 139-145 (1990)). Cyanogen bromide (CNBr) is one of the best methods for initial cleavage of proteins. CNBr cleaves proteins at the C-terminus of methionyl residues. Because the number of methionyl residues in proteins is usually low, CNBr usually generates a few large fragments. The reaction is usually performed in a 70% formic acid or 50% trifluoroacetic acid with a 50- to 100-fold molar excess of cyanogen bromide to methionine. Cleavage is usually quantitative in 10-12 hours, although the reaction is usually allowed to proceed for 24 hours. Some Met-Thr bonds are not cleaved, and cleavage can be prevented by oxidation of methionines.

Proteins can also be cleaved using partial acid hydrolysis methods to remove single terminal amino acids (Vanfleteren et al., BioTechniques 12: 550-557 (1992). Peptide bonds containing aspartate residues are particularly susceptible to acid cleavage on either side of the aspartate residue, although usually quite harsh conditions are needed. Hydrolysis is usually performed in concentrated or constant boiling hydrochloric acid in sealed tubes at elevated temperatures for various time intervals from 2 to 18 hours. Asp-Pro bonds can be cleaved by 88% formic acid at 37°. Asp-Pro bonds have been found to be susceptible under conditions where other Asp-containing bonds are quite stable. Suitable conditions are the incubation of protein (at about 5 mg/ml) in 10% acetic acid, adjusted to pH 2.5 with pyridine, for 2 to 5 days at 40° C.

Brominating reagents in acidic media have been used to cleave polypeptide chains. Reagents such as N-bromosuccinimide will cleave polypeptides at a variety of sites, including tryptophan, tyrosine, and histidine, but often give side reactions which lead to insoluble products. BNPS-skatole [2-(2-nitrophenylsulfenyl)-3-methylindole] is a mild oxidant and brominating reagent that leads to polypeptide cleavage on the C-terminal side of tryptophan residues.

Although reaction with tyrosine and histidine can occur, these side reactions can be considerably reduced by including tyrosine in the reaction mix. Typically, protein at about 10 mg/ml is dissolved in 75% acetic acid and a mixture of BNPSskatole and tyrosine (to give 100-fold excess over tryptophan and protein tyrosine, respectively) is added and incubated for 18 hours. The peptide-containing supernatant is obtained by centrifugation.

Apart from the problem of mild acid cleavage of Asp-Pro bonds, which is also encountered under the conditions of BNPS-skatole treatment, the only other potential problem is the fact that any methionine residues are converted to methioninesulfoxide, which cannot then be cleaved by cyanogen bromide. If CNBr cleavage of peptides obtained from BNPS-skatole cleavage is necessary, the methionine residues can be regenerated by incubation with 15% mercaptoethanol at 30° C. for 72 hours.

Treating proteins with o-Iodosobenzoic acid cleaves tryptophan-X bonds under quite mild conditions. Protein, in 80% acetic acid containing 4 M guanidine hydrochloride, is incubated with iodobenzoic acid (approximately 2 mg/ml of protein) that has been preincubated with p-cresol for 24 hours in the dark at room temperature. The reaction can be terminated by the addition of dithioerythritol. Care must be taken to use purified o-iodosobenzoic acid since a contaminant, o-iodoxybenzoic acid, will cause cleavage at tyrosine-X bonds and possibly histidine-X bonds. The function of p-cresol in the reaction mix is to act as a scavenging agent for residual o-iodoxybenzoic acid and to improve the selectivity of cleavage.

Two reagents are available that produce cleavage of peptides containing cysteine residues. These reagents are (2-methyl) N-1—benzenesulfonyl-N-4-(bromoacetyl)quinone diimide (otherwise known as Cyssor, for “cysteine-specific scission by organic reagent”) and 2-nitro-5-thiocyanobenzoic acid (NTCB). In both cases cleavage occurs on the amino-terminal side of the cysteine.

Incubation of proteins with hydroxylamine results in the fragmentation of the polypeptide backbone (Saris et al., Anal. Biochem. 132: 54-67 (1983). Hydroxylaminolysis leads to cleavage of any asparaginyl-glycine bonds. The reaction occurs by incubating protein, at a concentration of about 4 to 5 mg/ml, in 6 M guanidine hydrochloride, 20 mM sodium acetate+1% mercaptoethanol at pH 5.4, and adding an equal volume of 2 M hydroxylamine in 6 M guanidine hydrochloride at pH 9.0. The pH of the resultant reaction mixture is kept at 9.0 by the addition of 0.1 N NaOH and the reaction allowed to proceed at 45° C. for various time intervals; it can be terminated by the addition of 0.1 volume of acetic acid. In the absence of hydroxylamine, a base-catalyzed rearrangement of the cyclic imide intermediate can take place, giving a mixture of a-aspartylglycine and B-aspartylglycine without peptide cleavage.

There are many methods known in the art for hydrolysing protein by use of a proteolytic enzymes (Cleveland et al., J. Biol. Chem. 252: 1102-1106 (1977). All peptidases or proteases are hydrolases which act on protein or its partial hydrolysate to decompose the peptide bond. Native proteins are poor substrates for proteases and are usually denatured by treatment with urea prior to enzymatic cleavage. The prior art discloses a large number of enzymes exhibiting peptidase, aminopeptidase and other enzyme activities, and the enzymes can be derived from a number of organisms, including vertebrates, bacteria, fungi, plants, retroviruses and some plant viruses. Proteases have been useful, for example, in the isolation of recombinant proteins. See, for example, U.S. Pat. Nos. 5,387,518, 5,391,490 and 5,427,927, which describe various proteases and their use in the isolation of desired components from fusion proteins.

The proteases can be divided into two categories. Exopeptidases, which include carboxypeptidases and aminopeptidases, remove one or more amino terminal residues from polypeptides. Endopeptidases, which cleave within the polypeptide sequence, cleave between specific residues in the protein sequence. The various enzymes exhibit differing requirements for optimum activity, including ionic strength, temperature, time and pH. There are neutral endoproteases (such as Neutrase®) and alkline endoproteases (such as Alcalase® and Esperase®), as well as acid-resistant carboxypeptidases (such as carboxypeptidase-P).

There has been extensive investigation of proteases to improve their activity and to extend their substrate specificity (for example, see U.S. Pat. Nos. 5,427,927; 5,252,478; and 6,331,427 B1). One method for extending the targets of the proteases has been to insert into the target protein the cleavage sequence that is required by the protease. Recently, a method has been disclosed for making and selecting site-specific proteases (“designer proteases”) able to cleave a user-defined recognition sequence in a protein (see U.S. Pat. No. 6,383,775).

The different endopeptidase enzymes cleave proteins at a diverse selection of cleavage sites. For example, the endopeptidase renin cleaves between the leucine residues in the following sequence: Pro-Phe-His-Leu-Leu-Val-Tyr (SEQ ID NO: 5) (Haffey, M. L. et al., DNA 6:565 (1987). Factor Xa protease cleaves after the Arg in the following sequences: Ile-Glu-Gly-Arg-X (SEQ ID NO: 6); Ile-Asp-Gly-Arg-X (SEQ ID NO: 7); and Ala-Glu-Gly-Arg-X (SEQ ID NO: 8), where X is any amino acid except proline or arginine, (SEQ ID NOS: 6-8, respectively) (Nagai, K. and Thogersen, H. C., Nature 309:810 (1984); Smith, D. B. and Johnson, K. S. Gene 67:31 (1988)). Collagenase cleaves following the X and Y residues in following sequence: -Pro-X-Gly-Pro-Y-(where X and Y are any amino acid) (SEQ ID NO: 9) (Germino J. and Bastis, D., Proc. Natl. Acad. Sci. USA 81:4692 (1984)). Glutamic acid endopeptidase from S. aureus V8 is a serine protease specific for the cleavage of peptide bonds at the carboxy side of aspartic acid under acid conditions or glutamic acid alkaline conditions.

Trypsin specifically cleaves on the carboxy side of arginine, lysine, and S-aminoethyl-cysteine residues, but there is little or no cleavage at arginyl-proline or lysyl-proline bonds. Pepsin cleaves preferentially C-terminal to phenylalanine, leucine, and glutamic acid, but it does not cleave at valine, alanine, or glycine. Chymotrypsin cleaves on the C-terminal side of phenylalanine, tyrosine, tryptophan, and leucine. Aminopeptidase P is the enzyme responsible for the release of any N-terminal amino acid adjacent to a proline residue. Proline dipeptidase (prolidase) splits dipeptides with a prolyl residue in the carboxyl terminal position.

Ionization Fragmentation Cleavage of Peptides or Nucleic Acids

Ionization fragmentation of proteins or nucleic acids is accomplished during mass spectrometric analysis either by using higher voltages in the ionization zone of the mass spectrometer (MS) to fragment by tandem MS using collision-induced dissociation in the ion trap. (see, e.g., Bieman, Methods in Enzymology, 193:455-479 (1990)). The amino acid or base sequence is deduced from the molecular weight differences observed in the resulting MS fragmentation pattern of the peptide or nucleic acid using the published masses associated with individual amino acid residues or nucleotide residues in the MS.

Complete sequencing of a protein is accomplished by cleavage of the peptide at almost every residue along the peptide backbone. When a basic residue is located at the N-terminus and/or C-terminus, most of the ions produced in the collision induced dissociation (CID) spectrum will contain that residue (see, Zaia, J., in: Protein and Peptide Analysis by Mass Spectrometry, J. R. Chapman, ed., pp. 29-41, Humana Press, Totowa, N.J., 1996; and Johnson, R. S., et al., Mass Spectrom. Ion Processes, 86:137-154 (1988)). since positive charge is generally localized at the basic site. The presence of a basic residue typically simplifies the resulting spectrum, since a basic site directs the fragmentation into a limited series of specific daughter ions. Peptides that lack basic residues tend to fragment into a more complex mixture of fragment ions that makes sequence determination more difficult. This can be overcome by attaching a hard positive charge to the N-terminus. See, Johnson, R. S., et al., Mass Spectrom. Ion Processes, 86:137-154 (1988); Vath, J. E., et al., Fresnius Z Anal. Chem., 331:248-252 (1988); Stults, J. T., et al., Anal. Chem., 65:1703-1708 (1993); Zaia, J., et al., J. Am. Soc. Mass Spectrom., 6:423-436 (1995); Wagner, D. S., et al., Biol. Mass Spectrom., 20:419-425 (1991); and Huang, Z.-H., et al., Anal. Biochem., 268:305-317 (1999). The proteins can also be chemically modified to include a label which modifies its molecular weight, thereby allowing differentiation of the mass fragments produced by ionization fragmentation. The labeling of proteins with various agents is known in the art and a wide range of labeling reagents and techniques useful in practicing the methods herein are readily available to those of skill in the art. See, for example, Means et al., Chemical Modification of Proteins, Holden-Day, San Francisco, 1971; Feeney et al., Modification of Proteins: Food, Nutritional and Pharmacological Aspects, Advances in Chemistry Series, Vol. 198, American Chemical Society, Washington, D.C., 1982).

The methods described herein can be used to analyze target nucleic acid or peptide fragments obtained by specific cleavage as provided above for various purposes including, but not limited to, polymorphism detection, SNP scanning, bacteria and viral typing, pathogen detection, antibiotic profiling, organism identification, identification of disease markers, methylation analysis, microsatellite analysis, haplotyping, genotyping, determination of allelic frequency, multiplexing, nucleotide sequencing, re-sequencing and de novo sequencing.

C. Sequencing Techniques by Construction of a Sequencing Graph

As mentioned above, many de-novo sequencing procedures (i.e., without any a-priori information regarding the amplicon sequence under examination) are still performed based on the Sanger concept developed in 1977. However, this sequencing approach is often limited to sequences of length approximately 15 to 20 nucleotides (nts) when used with the aforementioned MALDI-TOF mass spectrometry. Other methods based on base-specific chemical cleavage have been developed as well, but have not been viable for the dramatically increased demand in DNA sequencing. A newly-developed sequencing machine using gel electrophoresis can determine a consecutive stretch of 300-500 bases. However, gel electrophoresis process may take more than four hours to determine those bases. In comparison, a mass spectrometry read can be performed in a few seconds, where the actual analysis time in terms of mass spectrometry is only nanoseconds to microseconds.

This section describes a method for combining base-specific cleavage reactions and mass spectrometry to perform de-novo sequencing capable of sequencing ‘long’ amplicon stretches (i.e., 200 or more nucleotides) with four or more cleavage experiments. The method includes obtaining an ‘arbitrary’ number of mass spectra from distinct base-specific cleavage experiments. The term ‘arbitrary’ means that the method described below is not limited to a certain number of experiments (like four experiments cleaving the four base nucleotides A, C, G, and T). For de-novo sequencing, however, it is preferable to perform four cleavage experiments, one for every base or, equivalently, two appropriate cleavage experiments on forward and reverse strand.

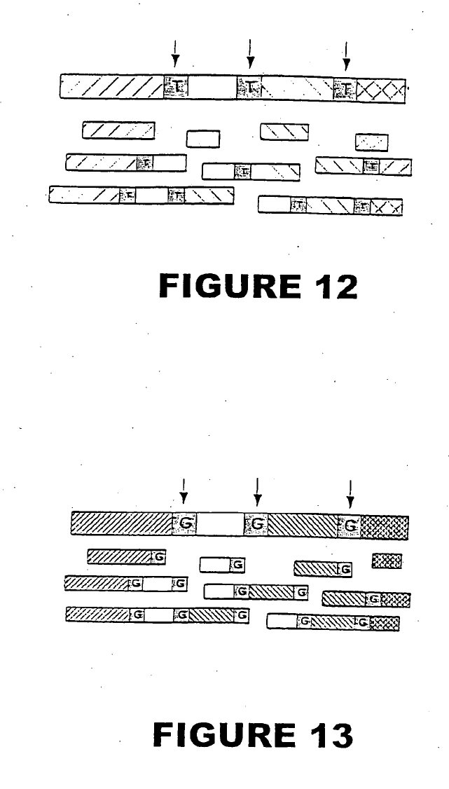

The cleavage experiments are performed with either partial cleavage or complete cleavage reactions. The mass spectra obtained only from complete cleavage reactions are often ambiguous even for short amplicon sequences of length 20 nts. For example, using four complete cleavage reactions (specific for each of the four bases), a differentiation between the spectra from sequences ACACCA and ACCACA (by searching for new or absent mass signals) is extremely difficult because even the intensities of mass signals are substantially similar. Thus, an amplicon sequence containing one of the above sequences as a sub-sequence cannot have a unique mass spectrum. A partial cleavage reaction is obtained by modifying the chemistry of the cleavage reaction such that only a certain percentage of the cut bases (i.e., the base(s) the cleavage reaction is specific to, such as T for UDG; see FIG. 12) is cleaved.

The ratio of cleaved versus un-cleaved cut bases can be adjusted such that mostly fragments containing none or one internal cut base will create a detectable peak. For example, a ratio of 70% cleaved versus 30% un-cleaved cut bases leads to predicted signal intensities of 0.49 for fragments with no internal cut base, 0.147 for one internal cut base, 0.0441 for two internal cut base, and 0.01323 or less for fragments containing three or more internal cut bases (where the intensity of a fragment peak from a complete cleavage experiment equals 1.0).

A ratio of 50:50 cleaved versus un-cleaved cut bases (instead of the ratio 70:30 proposed above) can be chosen when signal intensities and peak overlapping will allow such a ratio. This choice maximizes intensities of signals coming from fragments containing two internal cut bases and will henceforth be considered most appropriate for the analysis. In this case, relative intensities of mass signals will be 0.25, 0.125, 0.0625, and 0.03125 for fragments containing none, one, two, or three internal cut bases. Using mass spectrometry with high signal sensitivity, the first three signal types can be detected.

The method also includes extracting the ‘peak information’ from observed spectra. Initially, a differentiation between signal peaks and noise peaks in the spectrum is performed. Accordingly, a list of peaks (masses and intensities) for each spectrum is obtained, where masses and intensities can also be measured only up to some uncertainty.

Given that the amplicon sequence is known beforehand, the outcome for an arbitrary (complete or partial) cleavage reaction can be simulated to produce a list of predicted peaks. However, given a mass of the peak from a sample spectrum and the knowledge of the cleavage reaction, theoretical fragments (if any) that will create such a peak can be determined without any knowledge about the underlying amplicon sequence.

The method further includes applying a sequencing technique to the acquired data from the mass spectrometry. The application of the sequencing technique, described below in detail, includes transforming peak lists into a mathematical concept that can aid in reconstructing a sequence from fragments of a mass spectrum. This concept is referred to as a graph theory.

A graph is a mathematical construct composed of points in space called vertices and lines connecting the vertices called edges. Graphs can be used to model relationships across a set of objects, with each unit object represented by a vertex and each relationship between objects by an edge between vertices. Real-world situations can be represented by graphs, and graph theory techniques can provide solutions to problems that have been recast abstractly in terms of graphs.

In applying the graph theory to the sequencing problem, a sequencing graph G includes a set of vertices V and a set of edges E, where each edge connects either two vertices, or a vertex with itself. The term “sequencing graph”, as used herein, refers to a graph that attempts to represent the overall spatial arrangement of the fragments. In such a graph, two points are connected by an edge if they are, by a certain measure, closely related. The sequencing graph may also include a loop, which connects a vertex to itself. Thus, a sequencing graph can be built to represent cleaved sequence fragments as vertices and the adjacency of pairs of such fragments in the full nucleotide molecule as edges between appropriate vertices. However, since the ordering of base nucleotides within each fragment is not yet known, parameters referred to as compomers, which are different from ‘sequences’, are represented at the vertices.

The term “compomer” refers to the base composition of a sequence fragment, with the number n of each type of base B denoted by Bₙ. As stated above, since the order of bases in a fragment does not change the mass of the fragment (e.g., fragments ACG, AGC, CAG, CGA, GAC, and GCA have exactly the same mass), the fragments can be represented with compomers. Thus, the compomer containing ‘a’ adenine bases, ‘c’ cytosine bases, ‘g’ guanine bases, and ‘t’ thymine bases (in an unknown order) may be represented by AₐC_{c}G_{g}Tₜ. For the sake of brevity, A₀, C₀, G₀, and T₀ are usually omitted in this notation. For example, all of the above fragments, ACG, AGC, CAG, CGA, GAC, and GCA, can be represented by the unique compomer A₁C₁G₁.

The compomers may also be added as follows:
Aₐ₁C_{c1}G_{g1}Tₜ₁+Aₐ₂C_{c2}G_{g2}Tₜ₂=Aₐ₁₊ₐ₂C_{c1+c2}G_{g1+g2}Tₜ₁₊ₜ₂
For example, A₁C₅G₃+C₂G₃T₄ equals A₁C₇G₆T₄. In general, this is not equivalent to adding the masses of those compomers in a cleavage reaction. Further, a first compomer (e.g., c) includes a second compomer (e.g., c′) if, for any base B from A, C, G, and T, the number of bases in c is equal to or larger than the number of bases B in c′. For example, A₁C₂ is included in A₃C₂T₅, while the compomers A₁ and C₁ are exclusive of each other. A mathematical representation of mass spectrum of a compomer is described below.

Let s=s₁ . . . sₙ denote a string over the alphabet Σ where |s|=n denotes the length of s. In one example, the alphabet Σ:={A, C, G, T}. The concatenation of strings a, b will be denoted as ab, the empty string of length 0 is denoted as 0. If S=axb holds for some strings a, x,b then x is called a substring of s. We define the number of occurrence of x in s by:
#(x,s):=|{(a,b)ε(Σ^{*})²:s=axb}|
Hence, x is a substring of s if and only if #(x, s)≧1.

Given strings s and x from Σ^{*}, the string spectrum S(s,x) of s is defined by:
S(s,x):={s′εΣ^{*}: there exist a,bεΣ^{*} with sε{s′xb,axs′xb,axs′}}∪{s}.
Therefore, the string spectrum S(s, x) includes those substrings of s that are “bounded” by x (or the ends of s). In this context, s will be referred to as a sample string and x as a cut string, while the elements of S(s, x) will be referred to as fragments of s (under x).

As an example, consider the alphabet Σ:={0,A,C,G,T,1} where the characters 0, 1 are exclusively used to denote start and end of the sample string. For example, let s:=0ACATGTG1 and x:=T, then:
S(s,x)={0ACA,G,G1,0ACATG,GTG1,0ACATGTG1}.

As a mathematical representation of base compositions, a compomer is defined as a map c: Σ→N (where N denotes the set of natural numbers including zero). Furthermore, let C(Σ) denote the set of all compomers over the alphabet Σ. Thus, C(Σ) is closed with respect to addition as well as multiplications with a scalar nεN. For finite Σ, C(Σ) is isomorph to the set N|Σ|. The canonical partial order on C(Σ) is denoted by ≦, so that c≦c′ if and only if c(σ)≦c′(σ) for all σεΣ. Furthermore, the empty compomer c=0 is denoted by 0.

Suppose that Σ={σ₁, . . . , σₖ}, then the notation (σ₁)ᵢₗ, . . . (σₖ)ᵢ_{ₖ} is used to represent the compomer c: σⱼ [symbol] iⱼ omitting those characters σⱼ with iⱼ=0. In case of DNA, c represents the number of adenine, cytosine, guanine, and thymine bases in the compomer, and c=AᵢCⱼGₖTₗ denotes the compomer with c(A)=i, . . . , c(T)=l.

The function comp( ):Σ^{*}→C(Σ) is defined such that a string sεΣ^{*} is mapped to the compomer of s by counting the number of bases in s:
comp(s):Σ→N, σ [symbol] |{1≦i≦|s|:sᵢ=σ}|

The compomer spectrum C(s, x) of s includes the compomers of all fragments in the string spectrum:
C(s,x):=comp(S(s,x))
Hence, for the above-described example where s:=0ACATGTG1 and x:=T, it can be determined that
C(s,T)={0A₂C₁,G₁, G₁1,0A₂C₁G₁T₁, G₂T,1,0A₂C₁G₂T₂1}.

For an unknown string s and a known set of cleavage strings X, if there are characters that denote the start and end of the sample string (e.g., 0 and 1 to denote the start and end, respectively), then the unknown string s can be uniquely reconstructed from its compomer spectra C(s,x), xεX. Thus, for suitable X (e.g., X=Σ¹), the subsets {s′εC(s,x): s′₁=0} are sufficient to reconstruct s.

However, this approach will most likely fail when applied to experimental mass spectrometry data, because the theoretical approach of compomer spectra does not take into account the limitations of mass spectrometry and partial cleavage. Thus, these limitations imply that the probability that some fragment s′ cannot be detected, strongly depends on the multiplicity of the cut string x as a substring of s′. Moreover, signals from fragments with #(x,s′) above a certain threshold will most probably be lost in the noise of the mass spectrum.

As described above, in a compomer, the number of each type of base present is more important than the order in which those bases are arranged along the sequence. Since incomplete cleavage of nucleotide sequences is involved, it is possible to yield fragments containing a limited number of cut bases. The ‘order’ of the resulting directed sequencing graph, or the maximum number of cut bases that a fragment could have, is dependent on reaction conditions. Thus, all possible compomers having from zero to the ‘order’ number of cut bases need to be calculated before a sequencing graph can be built.

For example, all possible compomers with zero internal cut bases (i.e., order “0”) can be calculated for each peak in the mass spectrometry spectrum. Since a given peak in the mass spectrometry spectrum corresponds to a certain mass, computing all compomers with zero internal cut bases means finding all possible base compositions having no cut base, with theoretical masses that would equal that of the peak. The search is made within a margin of error set with a degree of predetermined mass uncertainty. It is assumed that a fragment with any such base composition might contribute to the peak.

All possible compomers with zero cut bases for all peaks can be calculated and put onto the undirected sequencing graph for a given cleavage reaction as vertices. Thus, each compomer having more than zero internal cut bases (i.e., higher than ‘0’ order) can be represented as a collection of smaller compomers separated by a cut base. The same type of calculation of compomers having zero internal cut bases can be repeated, where applicable, for compomers containing one cut base in their base composition, and so on.

Compomers are represented in the undirected sequencing graph not only as vertices, but also as edges connecting appropriate vertices. An edge is drawn between two vertices if that edge, a compomer, is the result of adding the compomers at the two vertices plus a cut base compomer, and the edge compomer has a mass where a peak was detected in the mass spectrum. The presence of a peak of an appropriate mass may indicate the existence of the compomer.

Construction of sequencing graphs is performed as follows: Once a list of peaks (masses and intensities) for each spectrum is obtained (referred to herein as “extracting peak information”), the list of peaks may be denoted by Pₙ for n=1, . . . , N where N is the number of cleavage experiments. For every cleavage experiment n=1, . . . ,N, a sequencing graph Gₙ=(Vₙ, Eₙ) can be constructed from the peak list Pₙ as follows. Initially, for every peak p with mass m in Pₙ, compomers c containing exactly zero cut bases are added to Vₙ if the predicted mass m_{c} of c is at most δₘ Dalton (Da) away from the measured mass m (i.e., |m−m_{c}|≦δₘ). A mass accuracy δₘ≧0 that depends on the applied mass spectrometry method may be chosen. Reasonable values can be selected from a range 0≦δₘ≦5. An empty compomer (denoted by the symbol ‘0’) can be added to Vₙ, as well as all compomers containing exactly one base to represent these compomers that cannot be detected in the mass spectrum due to mass range limitations.

For every peakp with mass m in Pₙ, compomers c containing exactly one cut base can then be added to a set of potential edges Ê such that the predicted mass m_{c} of c is at most δₘ Da away from the measured mass m. Also, let b denote the cut base of experiment n, and let c_{b} denote the compomer containing exactly one such cut base (i.e., c_{b} equals either A₁, C₁, G₁, or T₁). Next, define a set of edges Eₙ as a subset of Ê, where an element c in Ê is contained in Eₙ if and only if there exist vertices (compomers) v₁, v₂ in Vₙ such that c=v₁+c_{b}+v₂ holds. Finally, to include the information about the ‘first fragment’ to this graph, a starting vertex (denoted by a symbol ‘*’) and an edge, connecting the starting vertex with a compomer that corresponds to the start of an amplicon sequence to Eₙ, are added to Vₙ. In application, this compomer is either known a priori, because parts of the amplicon sequence are known, or it can be detected easily because all cleavage methods produce a known mass shift if a compomer corresponds to the start of an amplicon sequence.

In a particular embodiment, undirected sequencing graphs can be used to solve a sequencing-from-compomers (SFC) problem. This concept of using undirected sequencing graphs to solve an SFC problem is a special case of using the (more elaborate) directed sequencing graphs, which is described in detail below. For the sake of simplicity, the discussion in this section is limited to cut strings x of length one (i.e., the order of k=1). However, the concept can be extended to any arbitrary cut strings xεΣ.

An undirected graph G includes a set of vertices V, and a set of edges E⊂⊂²∪V, where an edge e with #e=1 is called a loop. It is assumed that such graphs are finite and, thus, have finite vertex set. A walk of G is a finite sequence of elements 9=(p₀,p₁, . . . ,pₙ) from V with {pᵢ₋₁, pᵢ} εE for all i=1, . . . ,n . Generally, p is not a path because p₀, . . . ,pₙ do not have to be pair-wise distinct. The number n=|p| is defined to be the length of p.

Given an arbitrary set of compomers C⊂C(Σ) and a single cut string xεΣ of length one, the undirected sequencing graph G(C, x)=(V, E) can be defined as follows: The vertex set V includes all compomers cεC such that c(x)=0 holds. The edge set E includes all compomers cεC such that c=u+comp(x)+v for some u, vεV holds. The vertices u, v are not required to be distinct in this equation. However, e(x)=1 must hold for all edges e of G(C,x).

As an example, consider Σ:={0,A,C,G,T,1}, s:=0CTAATCATAGTGCTG1, and x:=T. The compomer spectrum of order 1 can be determined as:

$${C}_{1} = C ( s , T , 1 ) = \{ \begin{matrix}0 {C}_{1} , 0 {A}_{2} {C}_{1} {T}_{1} , {A}_{2} , {A}_{3} {C}_{1} {T}_{1} , {A}_{1} {C}_{1} , {A}_{2} {C}_{1} {G}_{1} {T}_{1} , \\ {A}_{1} {G}_{1} , {A}_{1} {C}_{1} {G}_{2} {T}_{1} , {C}_{1} {G}_{1} , {C}_{1} {G}_{2} {T}_{1} 1 , {G}_{1} 1\end{matrix} \} .$$

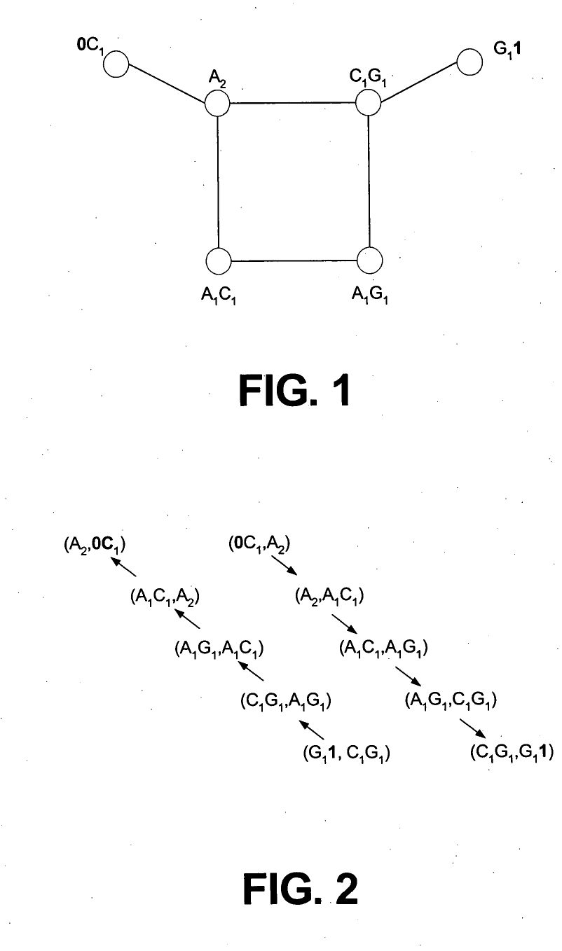

A corresponding undirected sequencing graph G₁ (C₁, T) is depicted in FIG. 1.

In another embodiment, directed graphs can be used to solve an SFC problem. A directed graph includes a set of vertices V and a set of edges E⊂V². An edge (v, v) for vεV is referred to as a loop. Again, it is assumed that the graphs are finite and, thus, have finite vertex set. A walk of G is a finite sequence of elements p=(p₀,p₁, . . . ,pₙ) from V with (pᵢ₋₁, Pᵢ) εE for all i=1, . . . ,n . The variable |p|=n denotes the length of p.

Given an alphabet Σ and order k, a graph Bₖ (Σ) (sometimes referred to as a de Bruijin Graph) is a directed graph with a vertex set V=Σ^{k} and an edge set
E={(u,v)εV²:uⱼ₊₁=vⱼ for all j=1, . . . ,k−1}
where u=(u₁, . . . ,uₖ) and v=(v₁, . . . ,vₖ). An edge ((e₁, . . . ,eₖ),(e₂, . . . ,eₖ₊₁)) of Bₖ(Σ) is sometimes denoted by (e₁, . . . ,eₖ₊₁) for short.

For an arbitrary set of compomers C⊂C(Σ) and a single cut string xεΣ of length one, the directed sequencing graph Gₖ (C,x) of order k can be defined as shown below.
Gₖ(C,x) is an edge-induced sub-graph of Bₖ(Σₓ) where
   Σₓ:={cεC:c(x)=0},
   and an edge e=(e₁, . . . ,eₖ₊₁) of Bₖ(Σₓ) belongs to Gₖ(C,x) if and only if the following condition holds:
   eᵢ+cₓ₊ₑᵢ₊₁+cₓ+ . . . +cₓ+eⱼ₋₁+cₓ+eⱼεC for all 1≦i≦j≦k+1
   Recall that cₓ denotes the compomer of the cut base x. Accordingly, by definition, the vertex set of Gₖ(C,x) is a subset of (Σₓ)^{k}

As an example, consider Σ:={0,A,C,G,T,1}, s:=0CTAATCATAGTGCTG1, and x:=T. The compomer spectrum of order 2 is:

$${C}_{2} = C ( s , T , 2 ) = \{ \begin{matrix}0 {C}_{1} , 0 {A}_{2} {C}_{1} {T}_{1} , 0 {A}_{3} {C}_{2} {T}_{2} , {A}_{2} , {A}_{3} {C}_{1} {T}_{1} , {A}_{4} {C}_{1} {G}_{1} {T}_{2} , {A}_{1} {C}_{1} , {A}_{2} {C}_{1} {G}_{1} {T}_{1} , \\ {A}_{2} {C}_{2} {G}_{2} {T}_{2} , {A}_{1} {G}_{1} , {A}_{1} {C}_{1} {G}_{2} {T}_{1} , {A}_{1} {C}_{1} {G}_{3} {T}_{2} 1 , {C}_{1} {G}_{1} , {C}_{1} {G}_{2} {T}_{1} 1 , {G}_{1} 1\end{matrix} \} .$$

A corresponding directed sequencing graph G₂ (C₂, T) is depicted in FIG. 2. Note that there are two paths connecting 0C₁ and G₁1 in the undirected sequencing graph G₂(Cᵢ, T), but only one directed walk from (0C₁, A₂) to (C₁G₁, G₁1) in the directed sequencing graph G₂(C₂, T).

In another example, if Σ:={0, A, B,1}, then the sample string s=0BABAAB 1 cannot be uniquely reconstructed from the complete cleavage compomer spectra C(s,x,0) for xε{A, B}, because the string s=0BAABAB 1 leads to the same spectra. Analogously, the string s=0BABABAABAB1 cannot be reconstructed from it compomer spectra C(s,x,1).

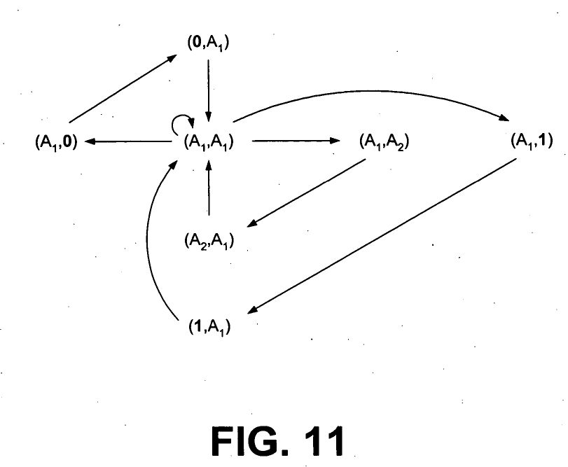

The graph G₂(C, B) for C(s, B,2) and s=0BABABABAABABAB1 produced analogously to above examples is shown in FIG. 11. If the non-relevant vertices (A₁,0) and (1,A₁) are removed, then there still exist two walks of length 6 from (0A₁,A₁) to (A₁, A₁1) that traverse all edges of the resulting graph. The two sequencing compatible with the two walks are s=0BABABABAABABAB1 and s=0BABABAABABABAB1.

A method for determining sequence information using compomers represented in a sequencing graph is mathematically described below. Sets of compomers Cₓ for xεX are given to solve the sequencing problem of finding all sample strings sεS⊂Σ^{*} satisfying C(s,x,k)⊂Cₓ for all xεX, where Σ denotes an alphabet, x=Σ^{*} denotes a set of cut strings, and kεN denotes a fixed order. These sets Cₓ were computed from the mass spectrum correlated to the cleavage reaction specific to x. Specifically, the directed sequencing graphs Gₖ(Cₓ,x) for xεX is constructed, and a mathematical concept referred to as a “walk” is performed to solve the sequencing problem. It may be assumed that the starting vertex v_{σ}^{start} and the ending vertex v_{σ}^{end} of the walk in graph Gₖ(C_{σ},σ) are known in advance for all cut bases σεΣ.

For Σₓ:={cεCₓ:c(x)=0}, all vectors (e₁, . . . ,eₖ₊₁)ε(Σₓ)^{k+1} that satisfy eᵢ+x+eᵢ₊₁+x+ . . . +x+eⱼ₋₁+x+eⱼεCₓ for all 1≦i≦j≦k+1 are searched. Every such vector e=(eᵢ, . . . ,eₖ₊₁) is added to the edge set of Gₖ(Cₓ,x), and (e₁, . . . ,eₖ) and (e₂, . . . , eₖ₊₁) are added to the vertex set of Gₖ(Cₓ, x). This can be performed in O(|Σₓ|^{k+1}k² log |Cₓ|) time.

In implementation, vertices and edges are added to the sequencing graph to achieve a single source and sink (i.e., start and end). The source vertices are of the form (*, . . . ,*,vₖ, . . . ,vₖ) where * ∉Σ denotes a special source character and 1<κ<k+1, and the source edges (e₁, . . . , eₖ₊₁) satisfy eⱼ=* for j<κ and eᵢ+x+eᵢ₊₁+x+ . . . +x+eⱼ₋₁+x+eⱼεC for all κ≦i≦j≦k+1. The vertex (*, . . . ,*) is then used in the resulting graph as the source vertex, and a sink can be built analogously. The sample string s and the current active vertices v_{σ} in Gₖ (C_{σ}, σ) for σεΣ are given. Further, s, denotes a unique string satisfying #(σ,s_{σ})=0 and s=s′_{σ}σs_{σ} for some s′_{σ}εΣ^{*}, and c_{σ}:=comp(s_{σ}).

A sequence candidate s is constructed by simultaneously constructing walks in the sequencing graphs Gₖ(C_{σ},σ) for all σεΣ according to the following conditions. If v_{σ}=v_{σ}^{end} for all σεΣ and |s|<lₘᵢₙ, then output s as a sequence candidate. Otherwise, if |s|<lₘₐₓ, then let Σₐ denote a set of “admissible” characters. For every admissible character xεΣₐ, a walk (recursion) is performed, where s is replaced by the concatenation sx, and the active vertex vₓ=(v₁, . . . ,vₖ) in Gₖ(Cₓ,x) is replaced by (v₂, . . . ,vₖ,cₓ), which is a vertex of the graph Gₖ (Cₓ,x). The parameters lₘᵢₙ and lₘₐₓ represent the minimal and the maximal length, respectively, for a sequence candidate. Here, a character xεΣ is designated as being “admissible” if the (k+1)-tuple (v₁, . . . ,vₖ,cₓ) is an edge of the sequencing graph Gₖ(Cₓ,x) given vₓ=(v₁, . . . ,vₖ) denotes the active vertex in Gₖ(Cₓ,x), and if there exists at least one edge (v₁, . . . ,vₖ,c′_{σ}) in the sequencing graph Gₖ(C_{σ},σ) such that c_{σ}+comp(x)≦c′_{σ} holds (i.e., the admissibility tests).

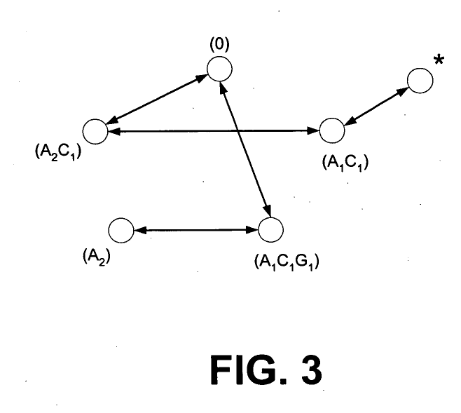

In using the above-described graph theory to perform sequencing, the following example illustrates an exemplary process of generating a sequencing graph shown in FIG. 3. In particular, a process for generating a directed sequencing graph G_{T} of order 1, which maps the cleavage reaction at thymine T (a cut base) with a sample sequence ACTACATTGACTAA (SEQ ID NO: 10), is illustrated. The compomers created by this cleavage experiment are AIC₁, A₂C₁, A₁C₁G₁, A2 (all containing no inner cut base), A₃C₂T₁, A₂C₁T₁, A₁C₁G₁T₁, A₃C₁G₁T₁ (all containing exactly one inner cut base), and further compomers with two or more inner cut bases (not shown). If it is assumed that all of these compomers create mass signals in our sample spectrum with a sufficiently small mass shift, then the vertex set of the graph would include the compomers with no inner cut base, empty compomers, and potentially other compomers due to peaks that misleadingly allow an interpretation as a compomer with no inner cut base. The empty compomers is denoted by symbol ‘0’, and the source vertex is denoted by symbol ‘*’. Empty compomer ‘0’ is added to the graph to account for twins of cut bases in the sample sequence. The source vertex ‘*’ indicates that the next compomer is a compomer that corresponds to the start of the amplicon sequence.

The set Ê is defined to include all compomers with exactly one inner cut base, plus potentially other compomers, which account for peaks known to be lost in the mass spectrum. Every ‘correct’ compomer in Ê will also be an edge of the graph, because any such compomer is made up of three sub-compomers: A compomer with no inner cut base, a cut base, and another compomer with no inner cut base. For example, in the sample sequence, A₃C₂T₁ equals A₁C₁+T₁+A₂C₁. Thus, under substantially optimal conditions, the graph G_{T} can be illustrated as shown in FIG. 3. In a sub-optimal condition, the graph might include more ‘misleading’ vertices and/or edges.

A ‘correct’ amplicon sequence can be obtained from the sequencing graph G_{T} as a walk within the graph. That is, given a sequence vᵢ, v₂, . . . , vₖ of vertices of G_{T}, vertices vⱼ and vⱼ₊₁ are connected by an edge (vⱼ,vⱼ₊₁) for all j=1, . . . , k−1. Thus, if a sequence does not correspond to a path in the sequencing graph, the sequence cannot be the ‘correct’ amplicon sequence. However, this criterion depends on not missing any signal peaks from fragments with zero/one inner cut base in the peak detection process.

The sequencing process also includes using all directed sequencing graphs G_{b} for bε{A, C, G, T} to reconstruct sequence candidates that might equal the sample sequence. If a sequence candidate is found, then further processing and testing may be applied. For simplicity, it is assumed that four proximity graphs G_{b}=G_{A}, G_{c}, G_{G}, and G_{T}, where G_{b} results from a cleavage experiment with a cutting base b.

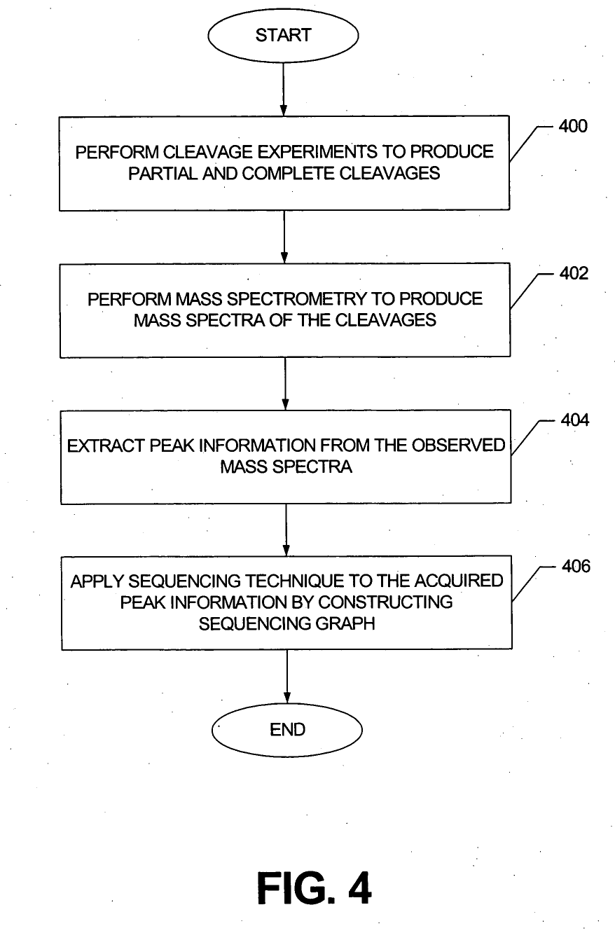

FIG. 4 is a flow diagram that illustrates an exemplary sequencing process that was described above. The process includes performing partial cleavage experiments, at box 400, to produce partial and complete cleavages or fragments. The cleavage experiments are performed by cleaving cut bases from the amplicon sequence. Preferably, four experiments are performed, one for every cut base (i.e., A, C, G, and T) or, equivalently, two appropriate cleavage experiments on forward and reverse strand. The cleavage experiments are performed with incomplete or partial cleavage reactions because the mass spectra obtained only from complete cleavage reactions are often extremely difficult to differentiate.

At box 402, mass spectrometry is performed to produce mass spectra of the acquired fragments. Peak information is extracted, at box 404, from the produced mass spectra, which includes performing differentiation between signal peaks and noise peaks in the spectrum. A list of peaks (masses and intensities) for each spectrum is then obtained.

It should be noted that the above process regarding the cleavage experiment and mass spectrometry is just an example illustrating the process of constructing a sequence graph. Other techniques well-known to those skilled in the art can be used.

The sequencing process also includes applying a sequencing technique to the acquired peak information, at 406. In an exemplary embodiment, the application of the sequencing technique includes constructing sequencing graphs and traversing these graphs in parallel, in a process referred to as a “walks”. The result of these “walks” is a candidate sequence that may be the sample sequence. The sequencing technique using sequencing graphs is further described in detail below.

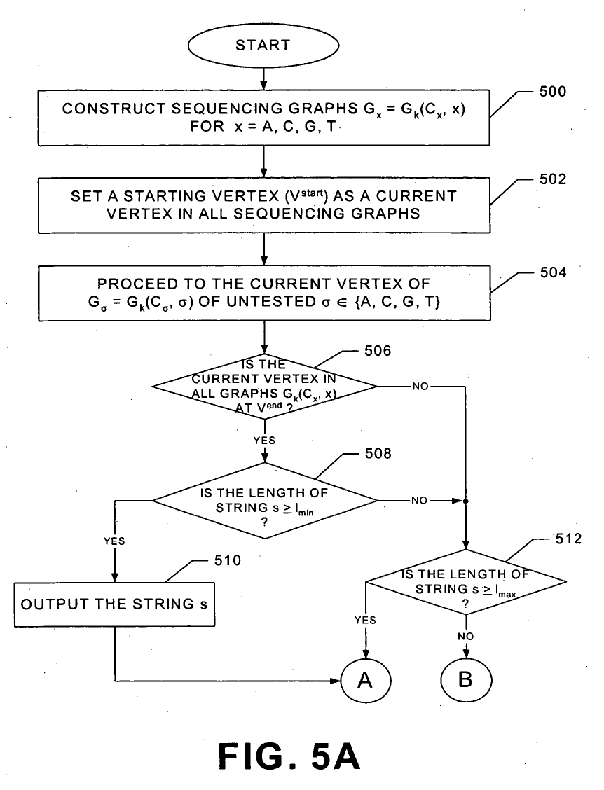

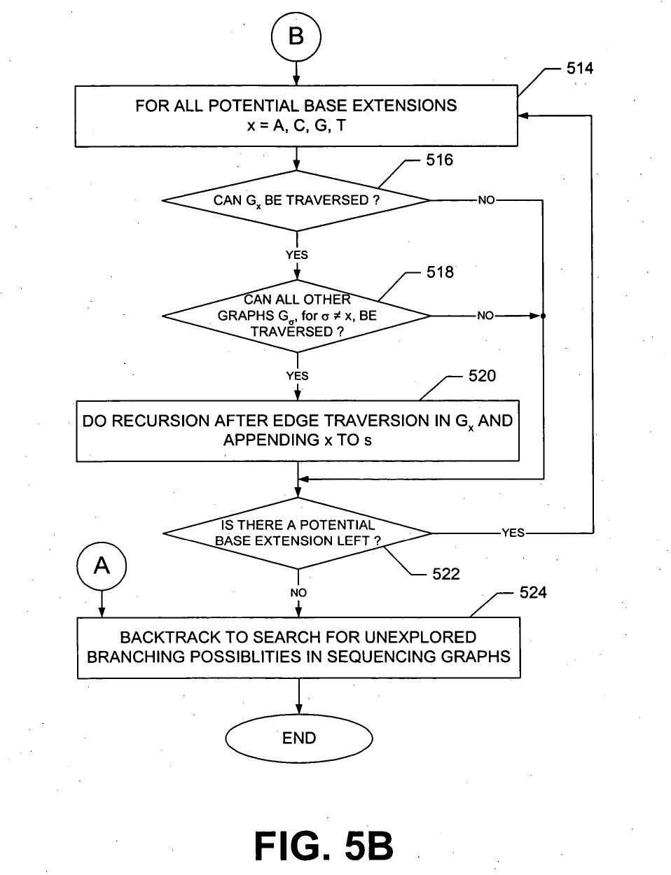

FIG. 5A and FIG. 5B form a flow diagram that illustrates an exemplary sequencing technique using sequencing graphs. In the exemplary embodiment, the sequencing technique involves constructing sequencing graphs Gₓ:=Gₖ(Cₓ, x) for bases x=A, C, G, and T, at box 500. A “walk” is then traced through each graph in all four graphs in parallel, starting at the source or starting vertex. A walk is an alternating sequence of vertices and edges, each edge being incident to the vertices immediately preceding and succeeding it. A walk does not imply special conditions, such as using each edge only once or visiting each vertex only once. To start the walk, the starting vertex (V^{start}) is set as a current vertex, at box 502, in all sequencing graphs. At box 504, the sequencing technique proceeds to the current vertex of the sequencing graph G_{σ}=Gₖ(C_{σ},σ) of untested cut base σεΣ, where σ={A, C, G,T}.

In each sequencing graph, successive connecting vertices are processed until the sink or ending vertex is reached in all sequencing graphs and the length of the reconstructed sequence has reached a threshold. These termination conditions are tested in boxes 506 and 508. Thus, if the current vertex in all sequencing graphs is at the ending vertex (V^{end}) (checked at box 506) and the length of the string s is greater than or equal to the predetermined minimal length (lₘᵢₙ) (checked at box 508), the string s is output as the candidate sequence, at box 510.

Otherwise, if the length of the string s is less than the predetermined maximal length (lₘₐₓ) (a “NO” outcome at the conditional box 512), a recursion in the sequencing technique is started, at box 514, for all potential base extensions x=A, C, G, and T. However, the sequencing technique cannot extend the current walk in a given graph, and thus cannot add a new base x, if either of the two following admissibility tests fail. Thus, if Gₓ cannot be traversed (checked at box 516), or one other graphs G_{σ}, for σ≈x, cannot be traversed in the future (checked at box 518), the recursion process is terminated, and the technique moves to box 522. The checked condition in the box 518 can be expressed as requiring at least one edge (v₁, . . . ,vₖ,c′_{σ}) in the sequencing graphs G_{σ}such that c_{σ}+comp(x)≦c′_{σ} holds. If both of the two admissibility tests (performed in boxes 516 and 518) pass, a recursion process is performed after traversing an edge in Gₓ, at box 520, and appending the base x to the string s representing the candidate sequence.

After determining that there are no more potential base extensions left (a “NO” outcome at box 522), the technique “backtracks” to search for unexplored branching possibilities in the sequencing graphs, at box 524. Otherwise, if there are more potential base extensions left (a “YES” outcome at box 522), the technique returns to box 514 to perform more recursion processes after additional admissibility tests. The term “backtracking” indicates an action where graphs are further explored by walking through alternate paths (i.e., alternate edges) from a previously-visited vertex. Thus, this technique is an example of a “branch-and-bound” problem, in which a solution can be found by tracing alternate paths from a different series of branches in a decision tree, constrained (‘bound’) by pre-specified conditions, until a solution meeting a set of requirements is found.

Since the sequencing technique presented above does not take into account all information present in the mass spectra, the technique will produce several candidate sequences that might be the correct sample sequence. For example, both peak intensities and mass shifts are neglected (only a threshold is applied). Accordingly, all candidate sequences determined by the sequencing technique can be further processed to resolve which of the candidates best explains the measured mass spectra. In one embodiment, a statistical analysis, such as a maximum likelihood test, can be performed to score the candidate sequences and determine the rank order of the fitness of the candidates to the measured mass spectra. In another embodiment, the candidate sequence can be checked to determine whether it includes the a priori “tail sequence” as a subsequence, and if the resulting sequence has appropriate length.

The procedure for building a sequencing graph, as well as the backtracking procedure, can be adapted to deal with 1½- and 2-cutters, as well as other cleavage techniques. An example of a 1½-cutter would be an enzyme that cleaves at every appearance of the bases CA and TA of the sample sequence. Moreover, using a 1½- or 2-cutter, in addition to the four 1-cutters, might increase the maximal length of an amplicon that can be sequenced successfully and, in addition, decrease the runtime of the sequencing technique. This is a result of the corresponding sequencing graph of a 1½- or 2-cutter being comparatively small and sparse (few vertices and edges) so that there are fewer sequence candidates. For example, an amplicon sequence of length 300 nts will lead to approximately 19 fragments with no inner cut base and 18 fragments with one inner cut base when cleaved with a 2-cutter, which is approximately one-fourth of the numbers expected for a 1-cutter.

To test the above-described sequencing process, artificial data, including a peak list, has been created by simulating a partial cleavage reaction with a computer and distorting the data by changing the expected mass by up to one Da. This peak list is then processed by a sequencing technique described above, which uses the sequencing graph. The amplicon sequence of length 80 nts (listed below (SEQ ID NO: 11)) was used.

AGAGTTTGAT CCTGGCTCAG GACGAACGCT GGCGGCGTGC
TTAACACATG CAAGTCGAAC GGAAAGGCCC CTTCGGGGGT.

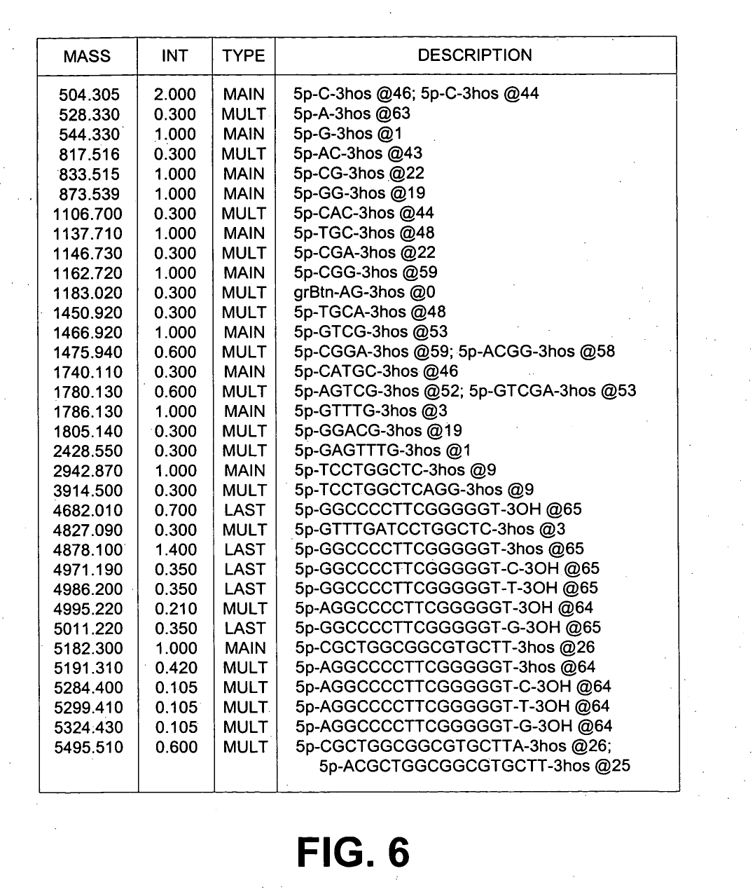

As an example, the construction of the sequencing graph for cut base A is illustrated. The expected list of peaks (with at most one internal cut base) is tabulated in FIG. 6. In practice, this list of peaks can be determined from the mass spectrum. The description column of the table also indicates starting positions of the detected compomers. For example, compomer ‘G’ detected at mass 544.33 is listed as starting at position ‘1’ and compomer ‘GTTTG’ detected at mass 1786.13 is listed as starting at position ‘3’. Thus, using the information tabulated in FIG. 6, an undirected sequencing graph (or equivalently, a directed sequencing graph of order 1) can be constructed, where the graph includes vertices indexed to compomers with no inner cut base and edges connecting those vertices. A determination as to which vertex would be connected to the current vertex by the current edge can be made by using the above-described condition of the vertices to be connected by the current edge.

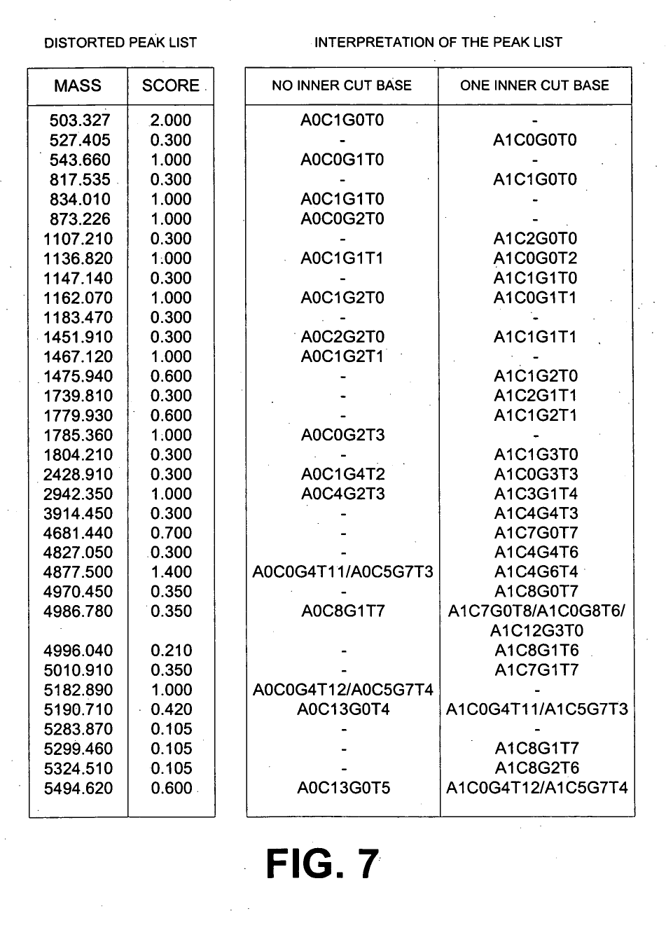

The distorted peak list is illustrated in the table on the left side of FIG. 7. Interpretation of the masses in the peak list as compomers with no inner cut base is shown in the left hand column of the table on the right side of FIG. 7. Interpretation of the masses as compomers with exactly one inner cut base is shown in the right hand column of the table on the right side of FIG. 7. The compomers are listed as corresponding to the masses listed in the distorted peak list.

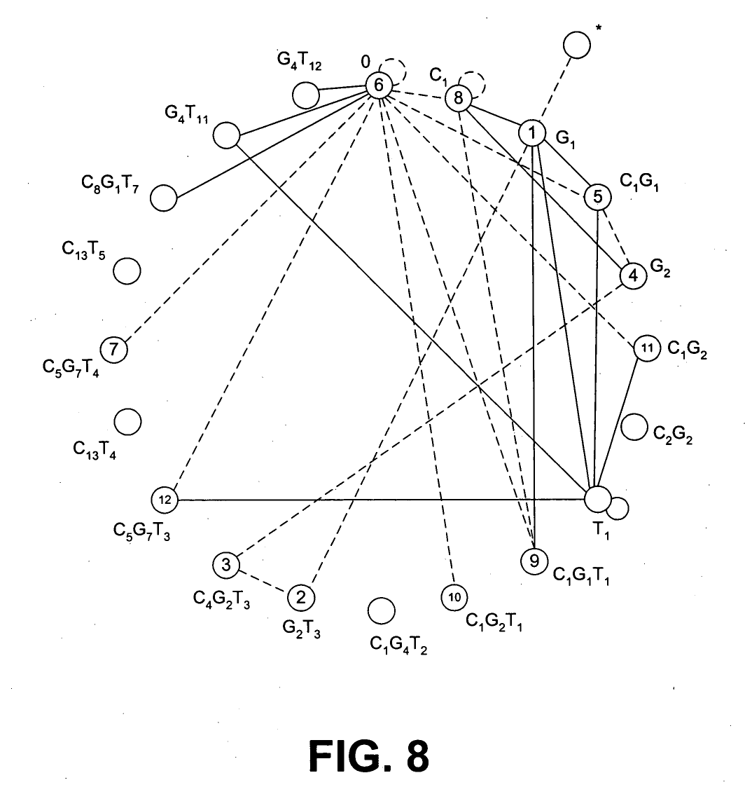

FIG. 8 shows a sequencing graph reconstructed from the compomers (edges of the path corresponding to the sample sequence are indicated by dashed and solid lines) interpreted from the peak list shown in FIG. 7. In particular, the dashed lines indicate that a walk can be found that corresponds to the input sequence. For the sake of brevity, the other three sequencing graphs (for cut bases C, G, and T) have been omitted. It is noted that tracing the dashed lines in the sequencing graph of FIG. 8 (sequentially tracing through the numbered vertices) corresponds to the correct sample sequence (of length 80 nts) listed above.

More specifically, the following shows how the correct sample sequence is constructed by the presented technique as one of the output sequences. In the illustrated embodiment of FIG. 8, the starting vertex with an empty compomer is indicated by an asterisk ‘*’. Since the table in the peak list of FIG. 6 indicates that a compomer having a value ‘G’ occupies the first position in the sequence, the starting vertex is connected to vertex #1 with compomer ‘G’. Thus, the current sequence s is equal to ‘A’ (edge from the starting vertex) plus ‘G’ (i.e., vertex #1), or ‘AG’. Next, a determination is made whether there is a connecting vertex. Since there is a connecting vertex (i.e., vertex #2), the vertex #1 is connected to the vertex #2 with an edge (i.e., a cut base A). A compomer with value ‘G₂T₃’ is indexed to vertex #2 because the table in FIG. 6 indicates that the compomer ‘G₂T₃’ at mass 1783.13 occupies third position in the sequence. Accordingly, the current vertex is set to vertex #2, and the current sequence s is set to the previous sequence (‘AG’) plus ‘A’ (an edge) plus ‘GTTTG’ (compomer value at vertex #2), which is equal to ‘AGAGTTTG’.

The above-described process for vertices #1 and #2 can be repeated for vertices #3 through #5 to determine that the current sequence s is equal to ‘AGAGTTTGATCCTGG CTCAGGACG’ (SEQ ID NO: 12). Vertex #6 is a vertex with an empty compomer. This allows vertex #6 to insert an edge to itself (i.e., a loop). Thus, vertex #6 inserts two edges (i.e., two ‘A’s), one connecting from vertex #5 and one connecting itself. Therefore, the current sequence s, after vertex #6, is equal to ‘AGAGTTTGATCCTGGCTCAGGACGAA’ (SEQ ID NO: 13).

The remaining vertices are traced (or “walked”) in sequence by repeating the process described above. However, there are some vertices that are visited more than once. Accordingly, the “walk” is taken in a sequence of vertices according to the table in FIG. 6, as follows: 1-2-3-4-5-6-6-7-6-6-8-8-9-6-6-10-6-6-11-6-6-6-12. Accordingly, by performing a “walk” according to this sequence of vertices, the sample sequence of 80 nts listed above can be sequenced from the sequencing graph shown in FIG. 8.

The described sequencing technique does not make use of peak intensity information obtained from mass spectrometry. In doing so, it might be possible to further increase sensitivity and specificity of the technique.

In the above sequencing technique, the processing of false negatives (i.e., missing peaks) is not fully addressed. Appropriate modifications to the sequencing technique to handle false negative data may be desirable. An exemplary modified technique is presented below.

The modified technique includes modifying the construction of the directed sequencing graph and the process of performing a “walk” through the graph. The modification of the construction of the directed graph includes constructing a weighted graph, where the weight of an edge represents an evaluation of the peaks missing in the spectrum. Thus, in one embodiment, the number of compomers (i.e., peaks) that are missing from the compomer spectrum (mass spectrum) is counted, and a determination can be made whether to add or not add an edge(s) to the sequencing graph based on comparison of the number of missing compomers with a threshold. The added edge can be weighted by the number of missing compomers.

In particular, the number of missing compomers can be represented as the number n of tuples (i, j) with 1≦i≦j≦k+1 such that
eᵢ+cₓ+eᵢ₊₁+cₓ+ . . . +cₓ+eⱼεCₓ holds.
If the number n does not exceed or is equal to a predefined threshold t₁, then an edge (e₁, . . . ,eₖ₊₁) is added to the graph Gₖ(Cₓ, x) with a weight of n. Otherwise, if the number n exceeds the threshold, then no edges are added.

In an alternative embodiment, a likelihood that a certain compomer eᵢ+cₓ+eᵢ₊₁+cₓ+ . . . +cₓ+eⱼ (and a corresponding peak) is missing from the compomer set Cₓ (and the mass spectrum) is calculated. By summing the negative log values of the likelihood calculation, a weighting function can be generated. Again, an edge(s) (e₁, . . . , eₖ₊₁) is added to the graph Gₖ(Cₓ,x) with weight w if the sum does not exceed or is equal to a predefined threshold.

In general, a penalizing function pₓ, which depends on the cleavage reaction, can be defined to map compomers into a set of real numbers. In one embodiment, this function is constant (i.e., p≡1) and, hence, only counts the number of missing compomers. For an edge (e₁, . . . ,eₖ₊₁), the weight can be defined as:
wₓ(e₁, . . . ,eₖ₊₁)=Σpₓ(eᵢ+x+eᵢ₊₁+x+ . . . +x+eⱼ),
where the function is summed over (i,j) for 1≦i≦j≦k+1 such that (e₁, . . . ,eₖ₊₁) is an edge of the sequencing graph, but eᵢ+x+eᵢ₊₁+x+ . . . +x+eⱼ∉C.

The sequencing technique is then modified as follows. A second threshold t₂ is chosen so that t₂ is in general larger than t₁. For the constant weighting derived from p≡1, this threshold t₂ represents a number of compomers (peaks) that are accepted as missing. A sum of the weights (denoted as w^{*}, and initialized to zero) is then tracked along with the sequence candidate generated by the recursion. That is, a character xεΣ is designated as being “admissible” if the admissibility tests pass and if the following condition holds. Let vₓ=(v₁, . . . ,vₖ) denote an active vertex in Gₖ(Cₓ,x). Then, the (k+1)-tuple (v₁, . . . ,vₖ,cₓ) must be an edge of the sequencing graph, and the total weight w^{*}+wₓ(v₁, . . . ,vₖ,cₓ) must not exceed the threshold t₂. Therefore, when the sequence candidate is generated by replacing s with the concatenation sx, the sum of the weights w is also replaced with w^{*}+wₓ(v₁, . . . ,vₖ,cₓ).

Accordingly, the resulting sequencing technique provides that any constructed sequence candidate s satisfy the following condition. For every cleavage character x, the expected compomer spectra Cₖ(s,x) is generated. Furthermore, let C′:=Cₖ(s,x)\Cₓ denote a set of false negative compomers, and let wₓ:=Σ_{cεC′}p(c) denote the sum of penalties. Then, Σ_{xεX}wₓ does not exceed the final sum of weights w^{*} corresponding to the constructed sequence candidate s and, hence, also does not exceed t₂. In fact, equality between Σ_{xεX}wₓ and w^{*} can be achieved by a suitable use of multi-sets instead of sets.

Some care has to be taken when choosing the threshold t₁. If the threshold t₁ is chosen to be too small, some sequence candidates that satisfy the above condition Σ_{xεX}wₓ≦t₂ may not be constructed by the technique. However, if the threshold t₁ is too large, the constructed sequencing graphs have many edges, which may result in increased runtimes.

D. Applications

As set forth herein, the methods provided herein are particular useful for de novo sequencing of target biomolecules, such as nucleic acids and polypeptides. The de novo sequencing methods provided herein are useful in a variety of applications. For example, if a polymorphism is identified or known, and it is desired to assess its frequency, the region of interest from different samples can be isolated, such as by PCR or restriction fragments, hybridization or other suitable method known to those of skill in the art and sequenced. For the methods provided herein, the de novo sequencing analysis is preferably effected using mass spectrometry (see, e.g., U.S. Pat. Nos. 5,547,835, 5,622,824, 5,851,765, and 5,928,906).

Once a de novo sequence is obtained using the methods provided herein, a variety of other applications become available to those of skill in the art by virtue of the newly acquired sequence information. Such exemplary applications are set forth hereinbelow in sections D.1-D.14.

1. Detection of Polymorphisms

An object herein is to provide improved methods for identifying the genomic basis of disease and markers thereof. The sequences identified by the methods provided herein include sequences containing sequence variations that are polymorphisms. Polymorphisms include both naturally occurring, somatic sequence variations and those arising from mutation. Polymorphisms include but are not limited to: sequence microvariants where one or more nucleotides in a localized region vary from individual to individual, insertions and deletions which can vary in size from one nucleotides to millions of bases, and microsatellite or nucleotide repeats which vary by numbers of repeats. Nucleotide repeats include homogeneous repeats such as dinucleotide, trinucleotide, tetranucleotide or larger repeats, where the same sequence in repeated multiple times, and also heteronucleotide repeats where sequence motifs are found to repeat. For a given locus the number of nucleotide repeats can vary depending on the individual.

A polymorphic marker or site is the locus at which divergence occurs. Such site can be as small as one base pair (an SNP). Polymorphic markers include, but are not limited to, restriction fragment length polymorphisms (RFLPs), variable number of tandem repeats (VNTR's), hypervariable regions, minisatellites, dinucleotide repeats, trinucleotide repeats, tetranucleotide repeats and other repeating patterns, simple sequence repeats and insertional elements, such as Alu. Polymorphic forms also are manifested as different mendelian alleles for a gene. Polymorphisms can be observed by differences in proteins, protein modifications, RNA expression modification, DNA and RNA methylation, regulatory factors that alter gene expression and DNA replication, and any other manifestation of alterations in genomic nucleic acid or organelle nucleic acids.

Furthermore, numerous genes have polymorphic regions. Since individuals have any one of several allelic variants of a polymorphic region, individuals can be identified based on the type of allelic variants of polymorphic regions of genes. This can be used, for example, for forensic purposes. In other situations, it is crucial to know the identity of allelic variants that an individual has. For example, allelic differences in certain genes, for example, major histocompatibility complex (MHC) genes, are involved in graft rejection or graft versus host disease in bone marrow transportation. Accordingly, it highly desirable to develop rapid, sensitive, and accurate methods for determining the identity of allelic variants of polymorphic regions of genes or genetic lesions. A method or a kit as provided herein can be used to genotype a subject by determining the identity of one or more allelic variants of one or more polymorphic regions in one or more genes or chromosomes of the subject. Genotyping a subject using a method as provided herein can be used for forensic or identity testing purposes and the polymorphic regions can be present in mitochondrial genes or can be short tandem repeats.

Single nucleotide polymorphisms (SNPs) are generally biallelic systems, that is, there are two alleles that an individual can have for any particular marker. This means that the information content per SNP marker is relatively low when compared to microsatellite markers, which can have upwards of 10 alleles. SNPs also tend to be very population-specific; a marker that is polymorphic in one population can not be very polymorphic in another. SNPs, found approximately every kilobase (see Wang et al. (1998) Science 280:1077-1082), offer the potential for generating very high density genetic maps, which will be extremely useful for developing haplotyping systems for genes or regions of interest, and because of the nature of SNPS, they can in fact be the polymorphisms associated with the disease phenotypes under study. The low mutation rate of SNPs also makes them excellent markers for studying complex genetic traits.

Much of the focus of genomics has been on the identification of SNPs, which are important for a variety of reasons. They allow indirect testing (association of haplotypes) and direct testing (functional variants). They are the most abundant and stable genetic markers. Common diseases are best explained by common genetic alterations, and the natural variation in the human population aids in understanding disease, therapy and environmental interactions.

2. Pathogen Typing

Provided herein is a process or method for identifying strains of microorganisms. The microorganism(s) are selected from a variety of organisms including, but not limited to, bacteria, fungi, protozoa, ciliates, and viruses. The microorganisms are not limited to a particular genus, species, strain, or serotype. The microorganisms can be identified by determining sequence variations in a target microorganism sequence relative to one or more reference sequences. The reference sequence(s) can be obtained from, for example, other microrganisms from the same or different genus, species strain or serotype, or from a host prokaryotic or eukaryotic organism. In another embodiment, the microrganisms can be identified by de novo sequencing according to the methods provided herein.

Identification and typing of bacterial pathogens is critical in the clinical management of infectious diseases. Precise identity of a microbe is used not only to differentiate a disease state from a healthy state, but is also fundamental to determining whether and which antibiotics or other antimicrobial therapies are most suitable for treatment. Traditional methods of pathogen typing have used a variety of phenotypic features, including growth characteristics, color, cell or colony morphology, antibiotic susceptibility, staining, smell and reactivity with specific antibodies to identify bacteria. All of these methods require culture of the suspected pathogen, which suffers from a number of serious shortcomings, including high material and labor costs, danger of worker exposure, false positives due to mishandling and false negatives due to low numbers of viable cells or due to the fastidious culture requirements of many pathogens. In addition, culture methods require a relatively long time to achieve diagnosis, and because of the potentially life-threatening nature of such infections, antimicrobial therapy is often started before the results can be obtained.

In many cases, the pathogens are very similar to the organisms that make up the normal flora, and can be indistinguishable from the innocuous strains by the methods cited above. In these cases, determination of the presence of the pathogenic strain can require the higher resolution afforded by the molecular typing methods provided herein. For example, PCR amplification of a target nucleic acid sequence followed by fragmentation by specific cleavage (e.g., base-specifc), followed by matrix-assisted laser desorption/ionization time-of-flight mass spectrometry, followed by screening for sequence variations once the de novo sequence is obtained by the methods provided herein, allows reliable discrimination of sequences differing by only one nucleotide and combines the discriminatory power of the sequence information generated with the speed of MALDI-TOF MS.

3. Detecting the Presence of Viral or Bacterial Nucleic Acid Sequences Indicative of an Infection

The methods provided herein can be used to determine the presence of viral or bacterial nucleic acid sequences indicative of an infection by identifying sequence variations that are present in the viral or bacterial nucleic acid sequences relative to one or more reference sequences. The reference sequence(s) can include, but are not limited to, sequences obtained from related non-infectious organisms, or sequences from host organisms. In another embodiment, the methods provided herein can be used to provide de novo sequence information of viruses or bacteria present in an infection.

Viruses, bacteria, fungi and other infectious organisms contain distinct nucleic acid sequences, including polymorphisms, which are different from the sequences contained in the host cell. A target DNA sequence can be part of a foreign genetic sequence such as the genome of an invading microorganism, including, for example, bacteria and their phages, viruses, fungi, protozoa, and the like. The processes provided herein are particularly applicable for distinguishing between different variants or strains of a microorganism in order, for example, to choose an appropriate therapeutic intervention. Examples of disease-causing viruses that infect humans and animals and that can be detected by a disclosed process include but are not limited to Retroviridae (e.g., human immunodeficiency viruses such as HIV-1 (also referred to as HTLV-III, LAV or HTLV-III/LAV; Ratner et. al., Nature, 313:227-284 (1985); Wain Hobson et. al., Cell, 40:9-17 (1985), HIV-2 (Guyader et. al., Nature, 328:662-669 (1987); European Patent Publication No. 0 269 520; Chakrabarti et. al., Nature, 328:543-547 (1987); European Patent Application No. 0 655 501), and other isolates such as HIV-LP (International Publication No. WO 94/00562); Picornaviridae (e.g., polioviruses, hepatitis A virus, (Gust et. al., Intervirology, 20:1-7 (1983)); enteroviruses, human coxsackie viruses, rhinoviruses, echoviruses); Calcivirdae (e.g. strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arenaviridae (hemorrhagic fever viruses); Reoviridae (e.g., reoviruses, orbiviruses and rotaviruses); Birnaviridae; Hepadnaviridae (Hepatitis B virus); Parvoviridae (parvoviruses); Papovaviridae; Hepadnaviridae (Hepatitis B virus); Parvoviridae (most adenoviruses); Papovaviridae (papilloma viruses, polyoma viruses); Adenoviridae (most adenoviruses); Herpesviridae (herpes simplex virus type 1 (HSV-1) and HSV-2, varicella zoster virus, cytomegalovirus, herpes viruses; Poxyiridae (variola viruses, vaccinia viruses, pox viruses); Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted, i.e., Hepatitis C); Norwalk and related viruses, and astroviruses.

Examples of infectious bacteria include but are not limited to Helicobacter pyloris, Borelia burgdorferi, Legionella pneumophilia, Mycobacteria sp. (e.g. M. tuberculosis, M. avium, M. intracellulare, M. kansaii, M. gordonae), Staphylococcus aureus, Neisseria gonorrheae, Neisseria meningitidis, Listeria monocytogenes, Streptococcus pyogenes (Group A Streptococcus), Streptococcus agalactiae (Group B Streptococcus), Streptococcus sp. (viridans group), Streptococcus faecalis, Streptococcus bovis, Streptococcus sp. (anaerobic species), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli.

Examples of infectious fungi include but are not limited to Cryptococcus neoformans, Histoplasma capsulatum, Coccidioides immitis, Blastomyces dermatitidis, Chlamydia trachomatis, Candida albicans. Other infectious organisms include protists such as Plasmodium falciparum and Toxoplasma gondii.

4. Antibiotic Profiling

The analysis of specific cleavage fragmentation patterns as provided herein improves the speed and accuracy of detection of nucleotide changes involved in drug resistance, including antibiotic resistance. Genetic loci involved in resistance to isoniazid, rifampin, streptomycin, fluoroquinolones, and ethionamide have been identified [Heym et al., Lancet 344:293 (1994) and Morris et al., J. Infect. Dis. 171:954 (1995)]. A combination of isoniazid (inh) and rifampin (rif) along with pyrazinamide and ethambutol or streptomycin, is routinely used as the first line of attack against confirmed cases of M. tuberculosis [Banerjee et al., Science 263:227 (1994)]. The increasing incidence of such resistant strains necessitates the development of rapid assays to detect them and thereby reduce the expense and community health hazards of pursuing ineffective, and possibly detrimental, treatments. The identification of some of the genetic loci involved in drug resistance has facilitated the adoption of mutation detection technologies for rapid screening of nucleotide changes that result in drug resistance.

5. Identifying Disease Markers

Provided herein are de novo sequencing methods for the rapid and accurate identification of sequence variations that are genetic markers of disease, which can be used to diagnose or determine the prognosis of a disease. Diseases characterized by genetic markers can include, but are not limited to, atherosclerosis, obesity, diabetes, autoimmune disorders, and cancer. Diseases in all organisms have a genetic component, whether inherited or resulting from the body's response to environmental stresses, such as viruses and toxins. The ultimate goal of ongoing genomic research is to use this information to develop new ways to identify, treat and potentially cure these diseases. The first step has been to screen disease tissue and identify genomic changes at the level of individual samples. The identification of these “disease” markers is dependent on the ability to detect changes in genomic markers in order to identify errant genes or polymorphisms. Genomic markers (all genetic loci including single nucleotide polymorphisms (SNPs), microsatellites and other noncoding genomic regions, tandem repeats, introns and exons) can be used for the identification of all organisms, including humans. These markers provide a way to not only identify populations but also allow stratification of populations according to their response to disease, drug treatment, resistance to environmental agents, and other factors.

6. Haplotyping

The methods provided herein can be used to detect haplotypes. In any diploid cell, there are two haplotypes at any gene or other chromosomal segment that contain at least one distinguishing variance. In many well-studied genetic systems, haplotypes are more powerfully correlated with phenotypes than single nucleotide variations. Thus, the determination of haplotypes is valuable for understanding the genetic basis of a variety of phenotypes including disease predisposition or susceptibility, response to therapeutic interventions, and other phenotypes of interest in medicine, animal husbandry, and agriculture.

Haplotyping procedures as provided herein permit the selection of a portion of sequence from one of an individual's two homologous chromosomes and to genotype linked SNPs on that portion of sequence. The direct resolution of haplotypes can yield increased information content, improving the diagnosis of any linked disease genes or identifying linkages associated with those diseases.

7. Microsatellites

The fragmentation-based methods provided herein allow for rapid, unambiguous detection of microsatellite sequences. Microsatellites (sometimes referred to as variable number of tandem repeats or VNTRs) are short tandemly repeated nucleotide units of one to seven or more bases, the most prominent among them being di-, tri-, and tetranucleotide repeats. Microsatellites are present every 100,000 bp in genomic DNA (J. L. Weber and P. E. Can, Am. J. Hum. Genet. 44, 388 (1989); J. Weissenbach et al., Nature 359, 794 (1992)). CA dinucleotide repeats, for example, make up about 0.5% of the human extra-mitochondrial genome; CT and AG repeats together make up about 0.2%. CG repeats are rare, most probably due to the regulatory function of CpG islands. Microsatellites are highly polymorphic with respect to length and widely distributed over the whole genome with a main abundance in non-coding sequences, and their function within the genome is unknown.

Microsatellites are important in forensic applications, as a population will maintain a variety of microsattelites characteristic for that population and distinct from other populations which do not interbreed.

Many changes within microsatellites can be silent, but some can lead to significant alterations in gene products or expression levels. For example, trinucleotide repeats found in the coding regions of genes are affected in some tumors (C. T. Caskey et al., Science 256, 784 (1992) and alteration of the microsatellites can result in a genetic instability that results in a predisposition to cancer (P. J. McKinnen, Hum. Genet. 175, 197 (1987); J. German et al., Clin. Genet. 35, 57 (1989)).

8. Short Tandem Repeats

The methods provided herein can be used to identify short tandem repeat (STR) regions in some target sequences of the human genome relative to, for example, reference sequences in the human genome that do not contain STR regions. STR regions are polymorphic regions that are not related to any disease or condition. Many loci in the human genome contain a polymorphic short tandem repeat (STR) region. STR loci contain short, repetitive sequence elements of 3 to 7 base pairs in length. It is estimated that there are 200,000 expected trimeric and tetrameric STRs, which are present as frequently as once every 15 kb in the human genome (see, e.g., International PCT application No. WO 9213969 A1, Edwards et. al., Nucl. Acids Res. 19:4791 (1991); Beckmann et. al. (1992) Genomics 12:627-631). Nearly half of these STR loci are polymorphic, providing a rich source of genetic markers. Variation in the number of repeat units at a particular locus is responsible for the observed polymorphism reminiscent of variable nucleotide tandem repeat (VNTR) loci (Nakamura et. al. (1987) Science 235:1616-1622); and minisatellite loci (Jeffreys et. al. (1985) Nature 314:67-73), which contain longer repeat units, and microsatellite or dinucleotide repeat loci (Luty et. al. (1991) Nucleic Acids Res. 19:4308; Litt et. al. (1990) Nucleic Acids Res. 18:4301; Litt et. al. (1990) Nucleic Acids Res. 18:5921; Luty et. al. (1990) Am. J. Hum. Genet 46:776-783; Tautz (1989) Nucl. Acid Res. 17:6463-6471; Weber et. al. (1989) Am. J. Hum Genet 44:388-396; Beckmann et. al. (1992) Genomics 12:627-631).

Examples of STR loci include, but are not limited to, pentanucleotide repeats in the human CD4 locus (Edwards el al., Nucl. Acid Res. 19:4791 (1991)); tetranucleotide repeats in the human aromatase cytochrome P-450 gene (CYP19; Polymeropoulos et al., Nucl. Acids Res. 19:195 (1991)); tetranucleotide repeats in the human coagulation factor XIII A subunit gene (F13A1; Polymeropoulos et. al., Nucl. Acids Res. 19:4306 (1991)); tetranucleotide repeats in the F13B locus (Nishimura et. al., Nucl. Acids Res. 20:1167 (1992)); tetranucleotide repeats in the human c-les/fps, proto-oncogene (FES; Polymeropoulos et. al., Nucl. Acids Res. 19:4018 (1991)); tetranucleotide repeats in the LFL gene (Zuliani et. al., Nucl. Acids Res. 18:4958 (1990)); trinucleotide repeats polymorphism at the human pancreatic phospholipase A-2 gene (PLA2; Polymeropoulos et. al., Nucl. Acids Res. 18:7468 (1990)); tetranucleotide repeats polymorphism in the VWF gene (Ploos et. al., Nucl. Acids Res. 18:4957 (1990)); and tetranucleotide repeats in the human thyroid peroxidase (hTPO) locus (Anker et. al., Hum. Mol. Genet. 1:137 (1992)).

9. Organism Identification

Polymorphic STR loci and other polymorphic regions of genes are sequence variations that are extremely useful markers for human identification, paternity and maternity testing, genetic mapping, immigration and inheritance disputes, zygosity testing in twins, tests for inbreeding in humans, quality control of human cultured cells, identification of human remains, and testing of semen samples, blood stains and other material in forensic medicine. Such loci also are useful markers in commercial animal breeding and pedigree analysis and in commercial plant breeding. Traits of economic importance in plant crops and animals can be identified through linkage analysis using polymorphic DNA markers. Efficient and accurate methods for determining the identity of such loci based on de novo sequencing methods are provided herein.

10. Detecting Allelic Variation

The methods provided herein allow for high-throughput, fast and accurate detection of allelic variants. Studies of allelic variation involve not only detection of a specific sequence in a complex background, but also the discrimination between sequences with few, or single, nucleotide differences. One method for the detection of allele-specific variants by PCR is based upon the fact that it is difficult for Taq polymerase to synthesize a DNA strand when there is a mismatch between the template strand and the 3′ end of the primer. An allele-specific variant can be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res., 18:999 [1990]).) The fragmentation-based methods provided herein overcome the limitations of the primer extension method.

11. Determining Allelic Frequency

The methods herein described are valuable for identifying one or more genetic markers whose frequency changes within the population as a function of age, ethnic group, sex or some other criteria. For example, the age-dependent distribution of ApoE genotypes is known in the art (see, Schächter et al. (1994) Nature Genetics 6:29-32). The frequencies of polymorphisms known to be associated at some level with disease can also be used to detect or monitor progression of a disease state. For example, the N291S polymorphism (N291S) of the Lipoprotein Lipase gene, which results in a substitution of a serine for an asparagine at amino acid codon 291, leads to reduced levels of high density lipoprotein cholesterol (HDL-C) that is associated with an increased risk of males for arteriosclerosis and in particular myocardial infarction (see, Reymer et al. (1995) Nature Genetics 10:28-34). In addition, determining changes in allelic frequency can allow the identification of previously unknown polymorphisms and ultimately a gene or pathway involved in the onset and progression of disease.

12. Epigenetics

The methods provided herein can be used to study variations in a target nucleic acid or protein relative to a reference nucleic acid or protein that are not based on sequence, e.g., the identity of bases or amino acids that are the naturally occurring monomeric units of the nucleic acid or protein. For example, the specific cleavage reagents employed in the methods provided herein may recognize differences in sequence-independent features such as methylation patterns, the presence of modified bases or amino acids, or differences in higher order structure between the target molecule and the reference molecule, to generate fragments that are cleaved at sequence-independent sites. Epigenetics is the study of the inheritance of information based on differences in gene expression rather than differences in gene sequence. Epigenetic changes refer to mitotically and/or meiotically heritable changes in gene function or changes in higher order nucleic acid structure that cannot be explained by changes in nucleic acid sequence. Examples of features that are subject to epigenetic variation or change include, but are not limited to, DNA methylation patterns in animals, histone modification and the Polycomb-trithorax group (Pc-G/tx) protein complexes (see, e.g., Bird, A., Genes Dev., 16:6-21 (2002)).

Epigenetic changes usually, although not necessarily, lead to changes in gene expression that are usually, although not necessarily, inheritable. For example, as discussed further below, changes in methylation patterns is an early event in cancer and other disease development and progression. In many cancers, certain genes are inappropriately switched off or switched on due to aberrant methylation. The ability of methylation patterns to repress or activate transcription can be inherited. The Pc-G/trx protein complexes, like methylation, can repress transcription in a heritable fashion. The Pc-G/trx multiprotein assembly is targeted to specific regions of the genome where it effectively freezes the embryonic gene expression status of a gene, whether the gene is active or inactive, and propagates that state stably through development. The ability of the Pc-G/trx group of proteins to target and bind to a genome affects only the level of expression of the genes contained in the genome, and not the properties of the gene products. The methods provided herein can be used with specific cleavage reagents that identify variations in a target sequence by de novo sequencing or by analyzing variations relative to a reference sequence that are based on sequence-independent changes, such as epigenetic changes.

13. Methylation Patterns

As set forth above, the de novo sequencing methods provided herein can be used to detect sequence variations that result from a change in methylation patterns in the target sequence. Analysis of cellular methylation is an emerging research discipline. The covalent addition of methyl groups to cytosine is primarily present at CpG dinucleotides (microsatellites). Although the function of CpG islands not located in promoter regions remains to be explored, CpG islands in promoter regions are of special interest because their methylation status regulates the transcription and expression of the associated gene. Methylation of promotor regions leads to silencing of gene expression. This silencing is permanent and continues through the process of mitosis. Due to its significant role in gene expression, DNA methylation has an impact on developmental processes, imprinting and X-chromosome inactivation as well as tumor genesis, aging, and also suppression of parasitic DNA. Methylation is thought to be involved in the cancerogenesis of many widespread tumors, such as lung, breast, and colon cancer, an in leukemia. There is also a relation between methylation and protein dysfunctions (long Q-T syndrome) or metabolic diseases (transient neonatal diabetes, type 2 diabetes).

Bisulfite treatment of genomic DNA can be utilized to analyze positions of methylated cytosine residues within the DNA. Treating nucleic acids with bisulfite deaminates cytosine residues to uracil residues, while methylated cytosine remains unmodified. Thus, by comparing the sequence of a target nucleic acid that is not treated with bisulfite with the sequence of the nucleic acid that is treated with bisulfite in the methods provided herein, the degree of methylation in a nucleic acid as well as the positions where cytosine is methylated can be deduced.

Methylation analysis via restriction endonuclease reaction is made possible by using restriction enzymes which have methylation-specific recognition sites, such as Hpall and MSPI. The basic principle is that certain enzymes are blocked by methylated cytosine in the recognition sequence. Once this differentiation is accomplished, subsequent analysis of the resulting fragments can be performed using the methods as provided herein.

These methods can be used together in combined bisulfite restriction analysis (COBRA). Treatment with bisulfite causes a loss in BstUI recognition site in amplified PCR product, which causes a new detectable fragment to appear on analysis compared to untreated sample. The fragmentation-based methods provided herein can be used in conjunction with specific cleavage of methylation sites to provide rapid, reliable information on the methylation patterns in a target nucleic acid sequence.

14. Resequencing

The dramatically growing amount of available genomic sequence information from various organisms increases the need for technologies allowing large-scale comparative sequence analysis to correlate sequence information to function, phenotype, or identity. The application of such technologies for comparative sequence analysis can be widespread, including SNP discovery and sequence-specific identification of pathogens. Therefore, resequencing and high-throughput mutation screening technologies are critical to the identification of mutations underlying disease, as well as the genetic variability underlying differential drug response.

Several approaches have been developed in order to satisfy these needs. The current technology for high-throughput DNA sequencing includes DNA sequencers using electrophoresis and laser-induced fluorescence detection. Electrophoresis-based sequencing methods have inherent limitations for detecting heterozygotes and are compromised by GC compressions. Thus a DNA sequencing platform that produces digital data without using electrophoresis will overcome these problems. Matrix-assisted laser desorption/ionization time-of-flight mass spectrometry (MALDI-TOF MS) measures DNA fragments with digital data output. The de novo sequencing methods of specific cleavage fragmentation analysis provided herein allow for high-throughput, high speed and high accuracy in the detection of sequence variations relative to a reference sequence. This approach makes it possible to routinely use MALDI-TOF MS sequencing for accurate mutation detection, such as screening for founder mutations in BRCA1 and BRCA2, which are linked to the development of breast cancer.

15. Multiplexing

The de novo sequencing methods provided herein allow for the high-throughput detection or discovery of sequence variations in a plurality of target sequences relative to one or a plurality of reference sequences, or by de novo sequencing. Multiplexing refers to de-novo sequencing of several amplified sequences in a single set of reactions, or to the simultaneous detection of more than one polymorphism or sequence variation. For example, instead of sequencing a single DNA sequence of 200 nuncleotides, 10 separate DNA sequences of 20 nucleotides can be sequenced in parallel. Methods for performing multiplexed reactions, particularly in conjunction with mass spectrometry, are known (see, e.g., U.S. Pat. Nos. 6,043,031, 5,547,835 and International PCT application No. WO 97/37041).

Multiplexing can be performed, for example, for the same target nucleic acid sequence using different complementary specific cleavage reactions as provided herein, or for different target nucleic acid sequences, and the fragmentation patterns can in turn be analyzed against a plurality of reference nucleic acid sequences. Several mutations or sequence variations can also be simultaneously detected on one target sequence by employing the de novo sequencing methods provided herein where each sequence variation corresponds to a different cleavage fragment relative to the fragmentation pattern of the reference nucleic acid sequence.

16. Pooling

A mixture of biological samples from any two or more biomolecular sources can be pooled into a single mixture for analysis herein. For example, the methods provided herein can be used for sequencing multiple copies of a target nucleic or amino acids from different sources, and therefore detect sequence variations in a target nucleic or amino acid in a mixture of nucleic acids in a biological sample. A mixture of biological samples can also include but is not limited to nucleic acid from a pool of individuals, or different regions of nucleic acid from one or more individuals, or a homogeneous tumor sample derived from a single tissue or cell type, or a heterogeneous tumor sample containing more than one tissue type or cell type, or a cell line derived from a primary tumor. Also contemplated are methods, such as haplotyping methods, in which two mutations in the same gene are detected.

E. System and Software Method

Also provided are systems that automate the sequencing process using a computer programmed for identifying the candidate sequence based upon the methods provided herein. The methods herein can be implemented, for example, by use of the following computer systems and using the following calculations, systems and methods. An exemplary automated testing system includes a nucleic acid workstation that includes an analytical instrument, such as a gel electrophoresis apparatus or a mass spectrometer or other instrument for determining the mass of a nucleic acid molecule in a sample, and a computer for fragmentation data analysis capable of communicating with the analytical instrument (see, e.g., copending U.S. application Ser. Nos. 09/285,481, 09/663,968 and 09/836,629; see, also International PCT application No. WO 00/60361 for exemplary automated systems). In an exemplary embodiment, the computer is a desktop computer system, such as a computer that operates under control of the “Microsoft Windows” operation system of Microsoft Corporation or the “Macintosh” operating system of Apple Computer, Inc., that communicates with the instrument using a known communication standard such as a parallel or serial interface.

For example, systems for analysis of nucleic acid samples are provided. The systems include a processing station that performs a base-specific or other specific cleavage reaction as described herein; a robotic system that transports the resulting cleavage fragments from the processing station to a mass measuring station, where the masses of the products of the reaction are determined; and a data analysis system, such as a computer programmed to identify the de novo sequence information of the target nucleic acid sequence using the fragmentation data, that processes the data from the mass measuring station to identify a nucleotide or plurality thereof in a sample or plurality thereof. The system can also include a control system that determines when processing at each station is complete and, in response, moves the sample to the next test station, and continuously processes samples one after another until the control system receives a stop instruction.

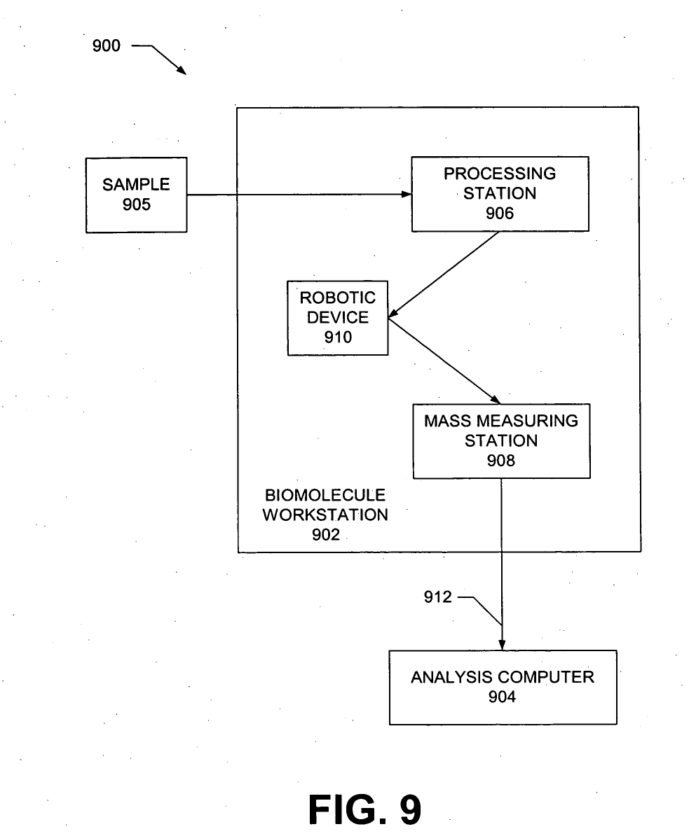

FIG. 9 is a block diagram of a system that performs sample processing and performs the operations illustrated in FIG. 4 and FIG. 5. The system 900 includes a biomolecule workstation 902 and an analysis computer 904. At the nucleic work station, one or more molecular samples 905 are received and prepared for analysis at a processing station 906, where the above-described cleavage reactions can take place. The samples are then moved to a mass measuring station 908, such as a mass spectrometer, where further sample processing takes place. The samples are preferably moved from the sample processing station 906 to the mass measuring station 908 by a computer-controlled robotic device 910.

The robotic device can include subsystems that ensure movement between the two processing stations 906, 908 that will preserve the integrity of the samples 905 and will ensure valid test results. The subsystems can include, for example, a mechanical lifting device or arm that can pick up a sample from the sample processing station 906, move to the mass measuring station 908, and then deposit the processed sample for a mass measurement operation. The robotic device 910 can then remove the measured sample and take appropriate action to move the next processed sample from the processing station 906.

The mass measurement station 908 produces data that identifies and quantifies the molecular components of the sample 905 being measured. Those skilled in the art will be familiar with molecular measurement systems, such as mass spectrometers, that can be used to produce the measurement data. The data is provided from the mass measuring station 908 to the analysis computer 904, either by manual entry of measurement results into the analysis computer or by communication between the mass measuring station and the analysis computer. For example, the mass measuring station 908 and the analysis computer 904 can be interconnected over a network 912 such that the data produced by the mass measuring station can be obtained by the analysis computer. The network 912 can comprise a local area network (LAN), or a wireless communication channel, or any other communications channel that is suitable for computer-to-computer data exchange.

The measurement processing function of the analysis computer 904 and the control function of the biomolecule workstation 902 can be incorporated into a single computer device, if desired. In that configuration, for example, a single general purpose computer can be used to control the robotic device 910 and to perform the data processing of the data analysis computer 904. Similarly, the processing operations of the mass measuring station and the sample processing operations of the sample processing station 906 can be performed under the control of a single computer.

Thus, the processing and analysis functions of the stations and computers 902, 904, 906, 908, 910 can be performed by variety of computing devices, if the computing devices have a suitable interface to any appropriate subsystems (such as a mechanical arm of the robotic device 910) and have suitable processing power to control the systems and perform the data processing.

The data analysis computer 904 can be part of the analytical instrument or another system component or it can be at a remote location. The computer system can communicate with the instrument can communicate with the instrument, for example, through a wide area network or local area communication network or other suitable communication network. The system with the computer is programmed to automatically carry out steps of the methods herein and the requisite calculations. For embodiments that use predicted fragmentation patterns (of a reference or target sequence) based on the cleavage reagent(s) and modified bases or amino acids employed, a user enters the masses of the predicted fragments. These data can be directly entered by the user from a keyboard or from other computers or computer systems linked by network connection, or on removable storage medium such as a data CD, minidisk (MD), DVD, floppy disk or other suitable storage medium. Next, the user initiates execution software that operates the system in which the sequencing graph is constructed and a walk is performed on the graph by tracing a path through vertices and edges of the graph.

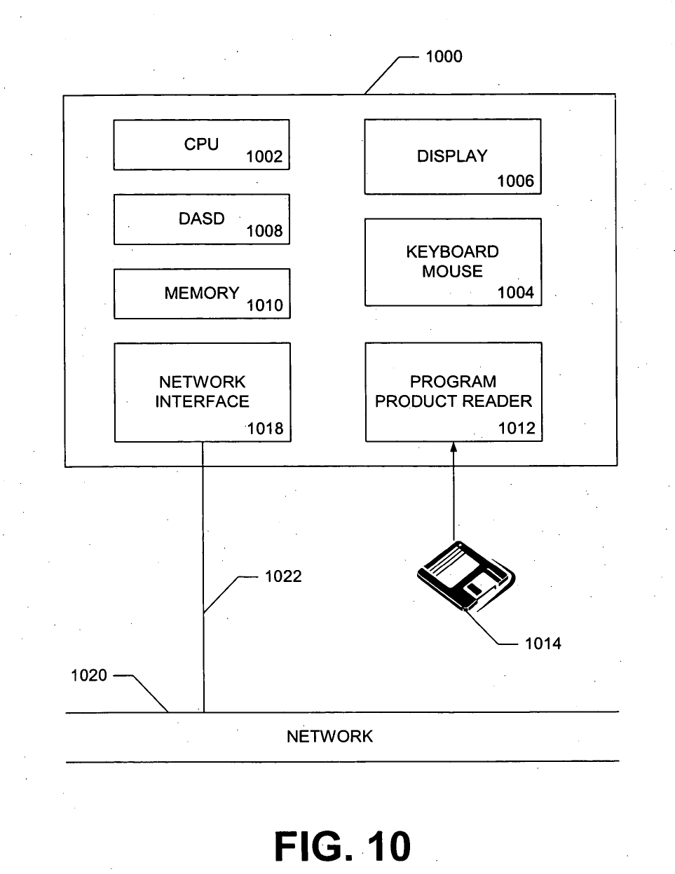

FIG. 10 is a block diagram of a computer in the system 900 of FIG. 9, illustrating the hardware components included in a computer that can provide the functionality of the stations and computers 902, 904, 906, 908. Those skilled in the art will appreciate that the stations and computers illustrated in FIG. 9 can all have a similar computer construction, or can have alternative constructions consistent with the capabilities and respective functions described herein. The FIG. 10 construction is especially suited for the data analysis computer 904 illustrated in FIG. 9.

FIG. 10 shows an exemplary computer 1000 such as might comprise a computer that controls the operation of any of the stations and analysis computers 902, 904, 906, 908. Each computer 1000 operates under control of a central processor unit (CPU) 1002, such as a “Pentium” microprocessor and associated integrated circuit chips, available from Intel Corporation of Santa Clara, Calif., USA. A computer user can input commands and data from a keyboard and computer mouse 1004, and can view inputs and computer output at a display 1006. The display is typically a video monitor or flat panel display. The computer 1000 also includes a direct access storage device (DASD) 1008, such as a hard disk drive. The computer includes a memory 1010 that typically comprises volatile semiconductor random access memory (RAM). Each computer preferably includes a program product reader 1012 that accepts a program product storage device 1014, from which the program product reader can read data (and to which it can optionally write data). The program product reader can comprise, for example, a disk drive, and the program product storage device can comprise removable storage media such as a magnetic floppy disk, a CD-R disc, a CD-RW disc, or DVD disc.

Each computer 1000 can communicate with the other FIG. 9 systems over a computer network 1020 (such as, for example, the local network 912 or the Internet or an intranet) through a network interface 1018 that enables communication over a connection 1022 between the network 1020 and the computer. The network interface 1018 typically comprises, for example, a Network Interface Card (NIC) that permits communication over a variety of networks, along with associated network access subsystems, such as a modem.

The CPU 1002 operates under control of programming instructions that are temporarily stored in the memory 1010 of the computer 1000. When the programming instructions are executed, the computer performs its functions. Thus, the programming instructions implement the functionality of the respective workstation or processor. The programming instructions can be received from the DASD 1008, through the program product storage device 1010, or through the network connection 1022. The program product storage drive 1012 can receive a program product 1014, read programming instructions recorded thereon, and transfer the programming instructions into the memory 1010 for execution by the CPU 1002. As noted above, the program product storage device can comprise any one of multiple removable media having recorded computer-readable instructions, including magnetic floppy disks and CD-ROM storage discs. Other suitable program product storage devices can include magnetic tape and semiconductor memory chips. In this way, the processing instructions necessary for operation in accordance with the methods and disclosure herein can be embodied on a program product.

Alternatively, the program instructions can be received into the operating memory 1010 over the network 1020. In the network method, the computer 1000 receives data including program instructions into the memory 1010 through the network interface 1018 after network communication has been established over the network connection 1022 by well-known methods that will be understood by those skilled in the art without further explanation. The program instructions are then executed by the CPU 1002 thereby comprising a computer process.

It should be understood that all of the stations and computers of the system 900 illustrated in FIG. 9 can have a construction similar to that shown in FIG. 10, so that details described with respect to the FIG. 10 computer 1000 will be understood to apply to all computers of the system 900. It should be appreciated that any of the communicating stations and computers can have an alternative construction, so long as they can communicate with the other communicating stations and computers illustrated in FIG. 9 and can support the functionality described herein. For example, if a workstation will not receive program instructions from a program product device, then it is not necessary for that workstation to include that capability, and that workstation will not have the elements depicted in FIG. 10 that are associated with that capability.

The following Examples are included for illustrative purposes only and are not intended to limit the scope of the invention.

### EXAMPLE 1

Base-Specific Cleavage of RNA

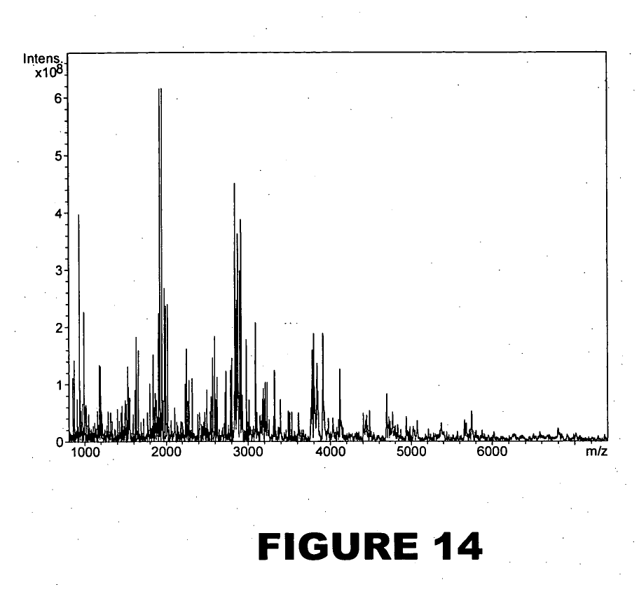

Provided herein is a semi-automated protocol for a one tube or multi-well reaction including RNA transcription and a T-specific endonucleolytic cleavage reaction with the exemplary RNAse, RNase A, to determine the de novo sequence of a target nucleic acid of interest. The fragments produced by the RNAse cleavage method as provided herein can be analyzed according to the methods provided herein. This partial cleavage produces a representative pattern of fragment masses as illustrated in FIG. 14, which using the algorithms provided herein is ultimately indicative of the sequence of a target sequence of interest. An exemplary protocol is provided below:

Materials and Methods

!PCR primer and amplicon sequence
Forward primer (SEQ ID NO: 14):
5′CAGTAATACGACTCACTATAGGGAGAAGGCTCCCCAGCAAGACGG
ACTT-3′
Reverse primer (SEQ ID NO: 15):
5′-AGGAAGAGAGCGCCTCGGCAAAGTACAC-3′
Amplicon (SEQ ID NO: 16):
5′-GGGAGAAGGC TCCCCAGCAA GACGGACTTC TTCAAAAACA
TCATGAACTT CATAGACATT GTGGCCATCA TTCCTTATTT
CATCACGCTG GGCACCGAGA TAGCTGAGCA GGAAGGAAAC
CAGAAGGGCG AGCAGGCCAC CTCCCTGGCC ATCCTCAGGG
TCATCCGCTT GGTAAGGGTT TTTAGAATCT TCAAGCTCTC
CCGCCACTCT AAGGGCCTCC AGATCCTGGG CCAGACCCTC
AAAGCTAGTA TGAGAGAGCT AGGGCTGCTC ATCTTTTTCC
TCTTCATCGG GGTCATCCTG TTTTCTAGTG CAGTGTACTT
TGCCGAGGCG CTCTCTTCCT-3′

PCR Protocol

The PCR reactions were set-up in 384 well MTP format with a total volume of 5 μl per well. The PCR mix comprised 1×HotStarTaq buffer (Qiagen, Hilden), 0.1 Unit of HotStarTaq DNA polymerase (Qiagen, Hilden), 200 μM of each dATP, dCTP, dTTP and dGTP, 5ng of genomic DNA, 200 nM of each, forward and reverse PCR primer.

The PCR mix was cycled with the following temperature profile: 15 min of enzyme activation at 94° C., followed by 45 amplification cycles (94° C. for 20 sec, 62° C. for 30 sec and 72° C. for 1 min.), followed by a final extension at 72° C. for 3 minutes, then stored at 4° C.

SAP Treatment to Remove Unicorporated dNTPs

To the 5 μl PCR products, a 2 μl reaction mix containing 1×HotStarTaq buffer (Qiagen, Hilden) and 0.3 Units of Shrimp Alkaline Phosphatase (SAP) was added and incubated for 20 min at 37C. The enzyme was inactivated by heating the reaction to 85C for 5 minutes.

RNA Transcription and RNase Cleavage

Each reaction utilizes 2 μl of transcription mix and 2 μl of the amplified DNA sample. For a T-specific cleavage, the transcription mix contains 40 mM Tris-acetate pH 8, 40 mM potassium actetate, 10 mM magnesium acetate, 8 mM spermidine, 1 mM each of ATP, GTP and UTP, 2.5 mM of dCTP, 5 mM of DTT and 20 units of T7 R&D polymerase (Epicentre). For T-specific partial cleavage, a respective 4:1 ratio (80:20 ratio) of dTTP to UTP is used. Transcription reactions were performed at 37° C. for 2 hours. Following transcription, 2 μl of RNase A (0.5 μg) was added to each transcription reaction. The RNase cleavage reactions were carried out at 370C for 1 hour.

Sample Conditioning and MALDI-TOF MS Analysis

Following RNase cleavage, each reaction mixture was diluted within a tube or 384-well plate by adding 20 μl of ddH₂O. Conditioning of the phosphate backbone was achieved by addition 6 mg of cation exchange resin (SpectroCLEAN, Sequenom) to each well, rotation for 5 min and centrifugation for 5 min at 640×g (2000 rpm, centrifuge IEC Centra CL3R, rotor CAT.244). Following centrifugation, 15 nl of sample was transferred to a SpectroCHIP® substrate using a piezoelectric pipette. Samples were analyzed on a Biflex linear TOF mass spectrometer (Bruker Daltonics, Bremen).

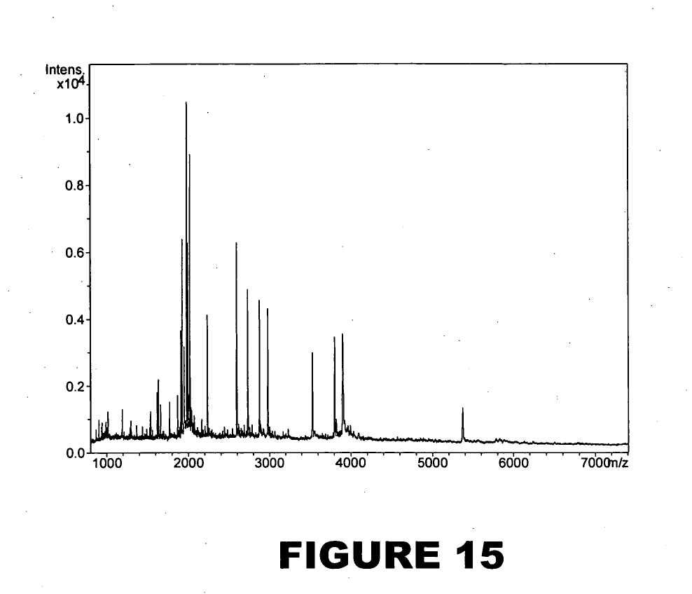

The resulting mass spectrum of RNase A cleavage mediated fragmentation of RNA transcripts for partial incomplete cleavage at every T using a 80:20 mixture of dTTP:rUTP is shown in FIG. 14, which can be compared to RNase A cleavage mediated fragmentation of RNA transcripts for complete cleavage using 100% dTTP as shown in FIG. 15.

### EXAMPLE 2

Base-Specific Cleavage of DNA

The following example describes a method for partially fragmenting a target nucleic acid according to the presence of a U residue in the nucleic acid, which is accomplished by digestion with the enzyme Uracil DNA glycosylase and phosphate backbone cleavage using NH₃. The fragmentation method provided herein can be used to generate base-specifically cleaved fragments of a target DNA, which can then be analyzed according to the methods provided herein to obtain the de novo sequence of the target DNA.

An exemplary protocol for partial cleavage is provided below: Reactions were carried out using a standard PCR amplicon and Uracil DNA Glycosylase mediated fragmentation. Two cleavage reactions were compared. A standard PCR was performed using 100% dUTP. In addition, a PCR with a 70:30 mixture of dUTP/dTTP was carried out.

!PCR primer and amplicon sequence
Forward primer (SEQ ID NO: 17):
5′-Bio CCCAGTCACGACGTTGTAAAACG-3′
Reverse Primer (SEQ ID NO: 18):
5′-AGCGGATAACAATTTCACACAGG-3′
Amplicon (SEQ ID NO: 19):
5′-CCCAGTCACG ACGTTGTAAA ACGTCCAGGG AGGACTCACC
ATGGGCATTT GATTGCAGAG CAGCTCCGAG TCCATCCAGA
GCTTCCTGCA GTCACCTGTG TGAAATTGTT ATCCGCT-3′

For partial incomplete cleavage, the DNA region of interest was amplified using PCR in the presence of a dUTP/dTTP mixture at a 70/30 ratio. The target region was amplified using a 50 μl PCR reaction containing 10 ng of genomic DNA, 1 unit of HotStarTaq DNA Polymerase (Qiagen), 0.2 mM each of dATP, dCTP and dGTP and 0.6 mM of dUTP in 1×HotStarTaq PCR buffer. PCR primers were used in asymmetric ratios of 5 pmol biotinylated primer and 15 pmol of non-biotinylated primer. The temperature profile program included 15 min of enzyme activation at 94° C., followed by 45 amplification cycles (95° C. for 30 sec, 56° C. for 30 sec and 72° C. for 30 sec), followed by a final extension at 72° C. for 5 min.

A comparison complete cleavage experiment was also conducted using 100% dUTP without any dTTP.

To achieve partial cleavage, 75 μg of Streptavidin Beads (Dynal, Oslo) were prewashed 2 times in 50 μl of 1×B/W buffer and resuspended in 45 μl of 2×B/W buffer (according to recommendation by manufacturer). Biotinylated PCR product was immobilized by adding the 50 μl PCR reaction to the resuspended Streptavidin Beads and incubation at room temperature for 20 min. The streptavidin beads carrying the immobilized PCR product were then incubated with 0.1 M NaOH for 5 min at room temperature to denature the double-stranded PCR product. After removal of the supernatant containing the non-biotinylated PCR strand, the beads were washed three times with 10 mM Tris-HCl pH 7.8 to neutralize the pH.

The beads were resuspended in 10 μl of UDG buffer (60 mM Tris-HCl pH 7.8, 1 mM EDTA pH 7.9), 2 units of Uracil DNA Glycosylase were added (MBI Fermentas) and the mixture was incubated at 37° C. for 45 minutes. Following the reaction, the beads were washed twice with 25 μl of 10 mM Tris-HCl pH 8, and once with 10 μl ddH₂O. The biotinylated strand was eluted by adding 12 μl of 500 mM NH₄OH and incubating at 60° C. for 10 min. After the 10 minute incubation, the supernatant was collected into a fresh microtiter plate or tube to cleave the phosphate at abasic sites, followed by incubation at 95° C. for 10 minutes with a closed lid. To evaporate the ammonia, an incubation at 80° C. for 11 minutes is performed with an open lid.

Mass Spectrometric Analysis

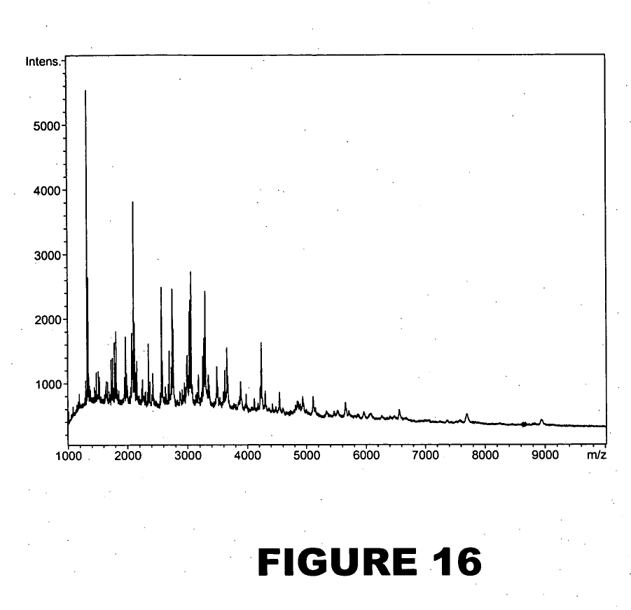

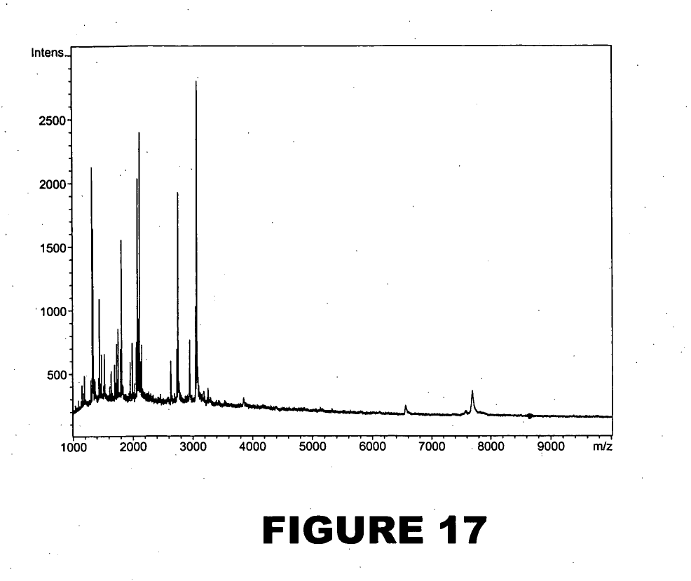

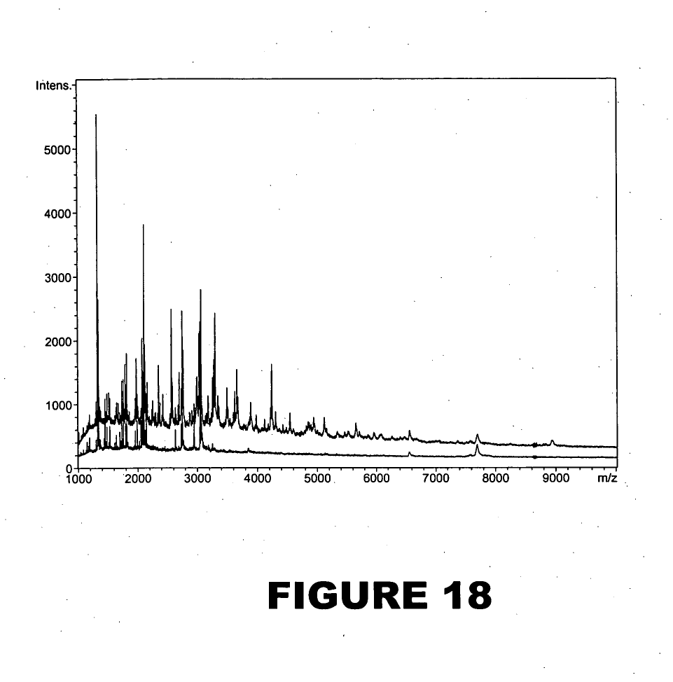

Following DNA cleavage, 15 nl of sample were transferred onto a SpectroCHIP® substrate (Sequenom) using a piezoelectric pipette. MALDI-TOF MS analysis was performed on a Bruker Bilex mass spectrometer (Bruker Daltonics, Bremen). The resulting mass spectrum of UDG mediated fragmentation: for incomplete cleavage using a 70:30 mixture of dUTP:dTTP is shown in FIG. 16; for complete cleavage using 100% dUTP is shown in FIG. 17; and of the overlay of the incomplete cleavage spectrum (upper spectrum) and the complete cleavage spectrum (lower spectrum) is shown in FIG. 18. As evident from the overlay of the two spectra, the use of a mixture of cleavable and non-cleavable nucleotides led to an increase in the number of fragments. Automated data analysis of the obtained mass signal pattern revealed that all calculated fragments containing none or exactly one inner cut-base could be identified in the case of incomplete cleavage, yielding the required sequence information necessary for exhaustive SNP discovery and de-novo sequencing.

### EXAMPLE 3

In this Example cleavage reactions were simulated and the performance of the algorithm described herein on the simulated data was examined. Two data sets were used to generate the sample DNA: The first data set corresponds to fragments of the human LAMBI gene (˜78,000 bases; ENSG00000091136; Reich et al, 2001, Nature, 411:199-204) were cut into approximately 400 pieces, each of length ˜200 bp. Each of the 200 base fragments was subjected to simulated cleavage reactions of order zero, one and two. The fragments containing zero, one or two uncleaved bases were then used to assemble the de novo sequence of each of the 200 bp fragments. The second data set contained random sample DNA sequences proposing that all bases have identical frequency ¼ of occurrence. In this embodiment for simulated fragments, approximately 1000 random sequences of length 200 bp each were analyzed in a manner similar to the analysis of the simulated fragments of the actual human LAMB1 gene.

For these simulations, an order k=2 was selected. Four cleavage reactions (based on “real world” RNAse cleavage) were simulated and only those fragments of order at most k were generated under the supposition that peaks from fragments of order k+1 and higher cannot be detected in the mass spectrum. Then, masses were calculated of all resulting fragments, and a limitation related to the calibration and resolution of the mass spectrometer was addressed in the following way: Assume that δ≧0 is the accuracy of the mass spectrometer, where δ is the maximal difference between an expected and the corresponding detected mass. For OTOF MS suppose δ=0.3 Da. Any signal from the expected list of peaks is perturbed so that its mass differs by at most δ from the expected mass, and for every resulting peak all compomers (of order at most k) that might possibly create a peak with mass at most δ off the perturbed signal mass are calculated. By this, the sets Cₓ for xεΣ are created. Note that the intensities of those peaks are not taken into account here. In addition, neither false positives (additional peaks) nor false negatives (missing peaks) are simulated here.

The sample DNA is reconstructed from the simulated cleavage reaction data using sequencing graphs of order k=2 and the algorithm presented herein. Note that for k=0 even short sample DNA cannot be uniquely reconstructed.

Results

Using the methods provided herein, for the random sequences, 96% of the 200 bp sequences were reconstructed with no error, while 99% of the sequences were reconstructed with up to two base errors. Thus, the error rate was about 0.4 per 1000 bp; For the actual fragments obtained by cleavage of the LAMB1 gene, 90% of the sequences were reconstructed with no error, while 96% of the sequences were reconstructed with up to two errors. Thus the error rate was about 2.5 per 1000 bp. As learned from these simulations, the most common sequencing error of this approach is the exchange of two bases belonging to a “stutter” repeat. As one could have expected, there were no sample sequences with exactly one ambiguous base.

Since modifications will be apparent to those of skill in this art, it is intended that this invention be limited only by the scope of the appended claims.

1. A method of obtaining sequence information from a target biomolecule, comprising:
fragmenting the target biomolecule into a plurality of fragments by partial cleavage;
performing mass spectrometry on the plurality of fragments to produce mass spectra of the fragments;
extracting peak information from the produced mass spectra;
constructing sequencing graphs using the extracted peak information; and
traversing the sequencing graphs to reconstruct the sequence information of the target biomolecule.

2. The method of claim 1, wherein constructing sequencing graphs includes generating a plurality of graphs having vertices and edges, each sequencing graph of the plurality of graphs representing a sequencing graph with a distinct cleavage reaction different from cleavage reactions used in other sequencing graphs of the plurality of graphs.

3. The method of claim 1, wherein each fragment of the plurality of fragments comprises a compomer.

4. The method of claim 3, wherein traversing the sequencing graphs includes tracing through each sequencing graph in the plurality of graphs, starting at a source vertex.

5. The method of claim 4, wherein traversing the sequencing graphs further includes setting the source vertex as a current vertex.

6. The method of claim 5, wherein traversing the sequencing graphs further includes setting a current sequence with the compomer of the current vertex.

7. The method of claim 6, wherein traversing the sequencing graphs further includes proceeding to the current vertex of the sequencing graph of an untested cleavage reaction.

8. The method of claim 7, wherein traversing the sequencing graphs further includes moving to a connecting vertex to the current vertex through an edge.

9. The method of claim 8, wherein traversing the sequencing graph further includes processing the connecting vertex.

10. The method of claim 9, wherein traversing the sequencing graphs further includes producing a candidate sequence by combining the traversed edge and vertex to the current sequence.

11. The method of claim 10, wherein traversing the sequencing graphs further includes determining whether the current vertex is an ending vertex.

12. The method of claim 11, wherein traversing the sequencing graphs further includes determining whether a length of the reconstructed sequence has reached a predetermined threshold.

13. The method of claim 12, wherein traversing the sequencing graphs further includes outputting the current sequence as a candidate sequence if the current vertex is the ending vertex and the length of the reconstructed sequence has reached the predetermined threshold.

14. The method of claim 12, wherein traversing the sequencing graphs further includes performing recursion after edge traversion if the current vertex is not the ending vertex.

15. The method of claim 12, wherein traversing the sequencing graphs further includes performing recursion after edge traversion if the length of the reconstructed sequence has not reached the predetermined threshold.

16. The method of claim 1, wherein traversing the sequencing graphs further includes backtracking to search for unexplored branching possibilities in the plurality of graphs.

17. A method for producing a candidate sequence of a biomolecule, comprising:
receiving a plurality of sequencing graphs, each sequencing graph having a plurality of vertices and edges, where each vertex represents a compomer of the biomolecule, and each edge represents a cut base of the sequencing graph; and
generating the candidate sequence by traversing the plurality of sequencing graphs.

18. The method of claim 17, further comprising:
traversing the plurality of sequencing graphs by tracing through each sequencing graph, starting at a source vertex.

19. The method of claim 18, wherein traversing the plurality of sequencing graphs includes setting the source vertex as a current vertex.

20. The method of claim 19, wherein traversing the plurality of sequencing graphs further includes setting the candidate sequence of the biomolecule as a compomer of the current vertex.

21. The method of claim 20, wherein traversing the plurality of sequencing graphs further includes proceeding to the current vertex of the sequencing graph of an untested cut base.

22. The method of claim 21, wherein traversing the plurality of sequencing graphs further includes moving to a connecting vertex from the current vertex through an edge.

23. The method of claim 22, wherein traversing the plurality of sequencing graphs further includes resetting the candidate sequence by appending compomers of the traversed edge and the connecting vertex to the previous-candidate sequence.

24. A program product for use in a computer that executes program instructions recorded in a computer-readable media to produce a candidate sequence of a biomolecule, the program product comprising:
a recordable medium; and
a plurality of computer-readable program instructions on the recordable media that are executable by the computer to perform a method comprising:
receiving a plurality of sequencing graphs, each sequencing graph having a plurality of vertices and edges, where each vertex represents a compomer of the biomolecule, and each edge represents a cut base of the sequencing graph; and
generating the candidate sequence by traversing the plurality of sequencing graphs.

25. The program product of claim 24, further comprising:
traversing the plurality of sequencing graphs by tracing through each sequencing graph, starting at a source vertex.

26. The program product of claim 25, wherein traversing the plurality of sequencing graphs includes setting the source vertex as a current vertex.

27. The program product of claim 26, wherein traversing the plurality of sequencing graphs further includes setting the candidate sequence of the biomolecule as a compomer of the current vertex.

28. The program product of claim 27, wherein traversing the plurality of sequencing graphs further includes proceeding to the current vertex of the sequencing graph of an untested cut base.

29. The program product of claim 28, wherein traversing the plurality of sequencing graphs further includes moving to a connecting vertex from the current vertex through an edge.

30. The program product of claim 29, wherein traversing the plurality of sequencing graphs further includes the candidate sequence by appending compomers of the traversed edge and the connecting vertex to the candidate sequence.

31. A sequencing system for obtaining sequence information from a target biomolecule, comprising:
a biomolecule workstation configured to process the target biomolecule into a plurality fragments and to produce mass spectra; and
an analysis computer configured to construct sequencing graphs using the mass spectra of the target biomelcule.

32. The system of claim 31, wherein the biomolecule workstation includes a processing station configured to receive and prepare one or more molecular samples for analysis.

33. The system of claim 32, wherein the processing station includes a cleaving element configured to provide for cleavage reactions on the one or more molecular samples to produce partially cleaved fragments.

34. The system of claim 33, wherein the biomolecule workstation includes a mass measuring station to perform mass spectrometry on the cleaved fragments.

35. The system of claim 34, wherein the biomolecule workstation includes a robotic device configured to move the molecular sample from the processing station to the mass measuring station.

36. The system of claim 35, wherein the robotic device includes a plurality of subsystems that ensure movement between the processing station and the mass measuring station to preserve the integrity of the samples.

37. The system of claim 36, wherein the plurality of subsystems include a mechanical lifting device to pick up the sample from the processing station and move the sample to the mass measuring station.

38. The system of claim 34, wherein the mass measuring station and the analysis computer are interconnected over a network.

39. The system of claim 38, wherein the network includes a local area network (LAN).

40. The system of claim 38, wherein the network includes a wireless communication channel.

41. The system of claim 38, wherein the network includes a wide area network (WAN).

42. The system of claim 41, wherein the wide area network (WAN) is the Internet.

43. The system of claim 31, wherein the analysis computer includes a neural network element to learn an efficient way to process the cleavages to obtain the sequence information of the target biomolecule.

44. A method of obtaining sequence information from a target biomolecule, comprising:
fragmenting the target biomolecule into at least two fragments by partial cleavage at specific cleavage sites;
determining the molecular weights of the at least two fragments;
determining the possible compositions of the at least two fragments;
ordering the possible compositions of the at least two fragments according to the number of specific cleavage sites that are not cleaved in each fragment;
constructing at least one sequencing graph that is a graph theoretical representation of the ordered compositions for the at least two fragments; and
traversing the at least one sequencing graph to reconstruct one or more underlying sequence candidates of the target biomolecule.

45. The method of claim 44, further comprising scoring the one or more underlying sequence candidates and determining the rank order of fitness.

46. The method of claim 45, wherein the scoring is done by statistical analysis.

47. The method of claim 46, wherein the scoring is done by maximum likelihood statistical analysis.

48. The method of claim 44 wherein the target biomolecule is DNA, and the compositions of the at least two fragments are the base compositions.

49. The method of claim 44, wherein the target biomolecule is RNA, and the compositions of the at least two fragments are the base compositions.

50. The method of claim 44, wherein the target biomolecule is a protein, and the compositions of the at least two fragments are the amino acid compositions.

51. The method of claim 44, wherein the molecular weights of the fragments are determined by mass spectrometry.

52. The method of claim 44, wherein the sequencing graph is a subgraph of a de Bruijn graph.

53. The method of claim 44, wherein the sequencing graph is traversed in a subgraph that is a walk.

54. A method of obtaining nucleic acid sequence information from a target nucleic acid molecule, comprising:
subjecting the nucleic acid molecule to partial cleavage reactions with one or more specific cleavage reagents, thereby generating two or more fragments that are specific cleavage products;
determining the molecular weights of the two or more fragments;
determining the possible base compositions of the two or more fragments;
ordering the possible base compositions of the two or more fragments according to the number of specific cleavage sites that are not cleaved in each fragment;
constructing one or more sequencing graphs that are graph theoretical representations of the ordered base compositions for the two or more fragments; and
traversing the one or more sequencing graphs to reconstruct one or more underlying sequence candidates, wherein each sequencing graph corresponds to the ordered base compositions derived from a partial cleavage reaction with one base-specific cleavage reagent.

55. The method of claim 54, wherein the one or more sequencing graphs are subgraphs of de Bruijn graphs that are traversed in a subgraph that is a walk.

56. The method of claim 54, wherein the nucleic acid molecule is subject to partial cleavage with two or more base-specific cleavage reagents and two or more sequencing graphs are constructed.

57. The method of claim 56, wherein the two or more sequencing graphs are traversed serially.

58. The method of claim 56, wherein the two or more sequencing graphs are traversed in parallel.

59. The method of claim 54, wherein the molecular weights of the two or more fragments are determined by mass spectrometry.

60. The method of claim 44, wherein the target biomolecule contains a sequence variation.

61. The method of claim 60, wherein the sequence variation is a mutation or a polymorphism.

62. The method of claim 61, wherein the mutation is an insertion, a deletion or a substitution.

63. The method of claim 61, wherein the polymorphism is a single nucleotide polymorphism.

64. The method of claim 44, wherein the target is a target nucleic acid molecule from an organism selected from the group consisting of eukaryotes, prokaryotes and viruses.

65. The method of claim 64, wherein the organism is a bacterium.

66. The method of claim 65, wherein the bacterium is selected from the group consisting of Helicobacter pyloris, Borelia burgdorferi, Legionella pneumophilia, Mycobacteria sp. (e.g. M. tuberculosis, M. avium, M. intracellulare, M. kansaii, M. gordonae), Staphylococcus aureus, Neisseria gonorrheae, Neisseria meningitidis, Listeria monocytogenes, Streptococcus pyogenes, Streptococcus agalactiae, Streptococcus sp., Streptococcus faecalis, Streptococcus bovis, Streptococcus pneumoniae, Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema palladium, Treponema pertenue, Leptospira and Actinomyces israelli.

67. The method of claim 44, wherein a specific cleavage reagent is an RNAse.

68. The method of claim 67, wherein a specific cleavage reagents are selected from among the RNase T₁, RNase U₂, the RNase PhyM, RNase A, chicken liver RNase (RNase CL3) and cusavitin.

69. The method of claim 44, wherein a specific cleavage reagent is a glycosylase.

70. The method of claim 44, wherein sequence variations in the target biomolecule permit genotyping a subject, forensic analysis, disease diagnosis or disease prognosis.

71. The method of claim 44, wherein the method determines epigenetic changes in a target nucleic acid molecule relative to a reference nucleic acid molecule.

72. A program product for use in a computer that executes program instructions recorded in a computer-readable media to obtain sequence information in a target biomolecule, the program product comprising:
a recordable medium; and
a plurality of computer-readable program instructions on the recordable media that are executable by the computer to perform a method comprising:
a) determining mass signals of target biomolecule fragments produced from partially cleaving a target biomolecule into fragments by contacting the target biomolecule with one or more base-specific cleavage reagents;
b) determining the possible compositions of the at least two fragments;
c) ordering the possible compositions of the at least two fragments according to the number of specific cleavage sites that are not cleaved in each fragment;
d) constructing at least one sequencing graph that is a graph theoretical representation of the ordered compositions for the at least two fragments; and
e) traversing the at least one sequencing graph to reconstruct one or more underlying sequence candidates of the target biomolecule.

73. The program product of claim 72, wherein the computer executable method further comprises scoring the candidate sequences and determining a rank order of sequence fitness.

74. The program product of claim 73, wherein determining a rank order of sequence fitness further comprises subjecting each of the target biomolecule candidate sequences to one or more statistical algorithms.

75. The program product of claim 72, wherein the masses are determined by mass spectrometry.

76. The method of claim 72, wherein the target biomolecule is a nucleic acid.

77. A combination of the program product of claim 72 and one or more specific cleavage reagents.

78. A system, comprising a computer, the program product of claim 72, and one or more specific cleavage reagents.

79. The combination of claim 77, further comprising:
one or more reference nucleic acid molecules; and/or one or more natural or modified nucleoside triphosphates.

80. A kit for determining de novo sequence information in one or more target nucleic acid molecules, comprising a combination of claim 77, and optionally instructions for determining de novo sequence information.

81. The kit of claim 80, wherein a specific cleavage reagent is an RNAse.

82. The kit of claim 81, wherein the RNAses are selected from among the RNase T₁, RNase U₂, the RNase PhyM, RNase A, chicken liver RNase (RNase CL3) and cusavitin.

83. A combination of the program product of claim 24 and one or more specific cleavage reagents.

84. A system, comprising a computer, the program product of claim 24, and one or more specific cleavage reagents.


SEQUENCE LISTING (19 sequences)

SEQ ID NO: 1 - DNA, 11 bp, Artificial Sequence
  UDG oligo
acatgtagcta

SEQ ID NO: 2 - DNA, 20 bp, Artificial Sequence
  cleavage fragment
aatgcacgtagccagtcaag

SEQ ID NO: 3 - DNA, 12 bp, Artificial Sequence
  cleavage fragment
gcacgtagccag

SEQ ID NO: 4 - DNA, 15 bp, Artificial Sequence
  cleavage fragment
aatgcacgtagccag

SEQ ID NO: 5 - protein, 7 aa, Artificial Sequence
  renin cleavage sequence
PFHLLVY

SEQ ID NO: 6 - protein, 5 aa, Artificial Sequence
  Factor Xa cleavage sequence
IEGRX

SEQ ID NO: 7 - protein, 5 aa, Artificial Sequence
  Factor Xa cleavage sequence
IDGRX

SEQ ID NO: 8 - protein, 5 aa, Artificial Sequence
  Factor Xa cleavage sequence
AEGRX

SEQ ID NO: 9 - protein, 5 aa, Artificial Sequence
  Collagenase cleavage sequence
PXGPX

SEQ ID NO: 10 - DNA, 14 bp, Artificial Sequence
  sample sequence
actacattgactaa

SEQ ID NO: 11 - DNA, 80 bp, Artificial Sequence
  amplicon sequence
agagtttgatcctggctcaggacgaacgctggcggcgtgcttaacacatgcaagtcgaacggaaaggccccttcgggggt

SEQ ID NO: 12 - DNA, 24 bp, Artificial Sequence
  sequence s
agagtttgatcctggctcaggacg

SEQ ID NO: 13 - DNA, 26 bp, Artificial Sequence
  sequence s
agagtttgatcctggctcaggacgaa

SEQ ID NO: 14 - DNA, 49 bp, Artificial Sequence
  forward primer
cagtaatacgactcactatagggagaaggctccccagcaagacggactt

SEQ ID NO: 15 - DNA, 28 bp, Artificial Sequence
  reverse primer
aggaagagagcgcctcggcaaagtacac

SEQ ID NO: 16 - DNA, 340 bp, Artificial Sequence
  amplicon
gggagaaggctccccagcaagacggacttcttcaaaaacatcatgaacttcatagacattgtggccatcattccttatttcatcacgctgggcaccgagatagctgagcaggaaggaaaccagaagggcgagcaggccacctccctggccatcctcagggtcatccgcttggtaagggtttttagaatcttcaagctctcccgccactctaagggcctccagatcctgggccagaccctcaaagctagtatgagagagctagggctgctcatctttttcctcttcatcggggtcatcctgttttctagtgcagtgtactttgccgaggcgctctcttcct

SEQ ID NO: 17 - DNA, 23 bp, Artificial Sequence
  forward primer
cccagtcacgacgttgtaaaacg

SEQ ID NO: 18 - DNA, 23 bp, Artificial Sequence
  reverse primer
agcggataacaatttcacacagg

SEQ ID NO: 19 - DNA, 117 bp, Artificial Sequence
  amplicon
cccagtcacgacgttgtaaaacgtccagggaggactcaccatgggcatttgattgcagagcagctccgagtccatccagagcttcctgcagtcacctgtgtgaaattgttatccgct